(19) United States
(12) Patent Application Publication
(10) Pub. No.: US 2017/0018717 A1
(43) Pub. Date: Jan. 19, 2017

(54) NEW CYCLAZINES AND THEIR USE AS SEMICONDUCTORS

(71) Applicants: BASF SE, Ludwigshafen (DE); Max-Planck-Gesellschaft Zur Förderung Der Wissenschaften e. V., München (DE)

(72) Inventors: THOMAS GESSNER, Heidelberg (DE); Helmut REICHELT, Neustadt (DE); Thomas WEITZ, Mannheim (DE); Michael EUSTACHI, Walldort (DE); Daniel JAENSCH, Mainz (DE); Long CHEN, Mainz (DE); Artem N. SKABEEV, Mainz (DE); Klaus MUELLEN, Köln (DE); Hans REICHERT, Rheinfelden (DE)

(21) Appl. No.: 15/120,647

(22) PCT Filed: Feb. 24, 2015
(86) PCT No.: PCT/IB2015/051361

§ 371 Date: Aug. 22, 2016

(30) Foreign Application Priority Data
Feb. 24, 2014 (EP) 14156298.3
Nov. 26, 2014 (EP) 14194977.6

(51) Int. Cl. H01L 51/00; C07D 471/16; C07D 471/22; C08K 5/3437

(52) U.S. Cl. CPC H01L 51/0053; C08K 5/3437; C07D 471/16; C07D 471/22; H01L 51/0558

(57) ABSTRACT

The present invention relates to compounds of the formula I

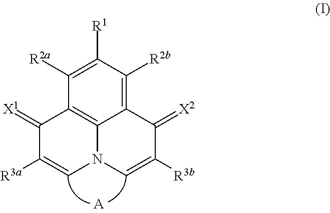

wherein X¹, X² are O or C(CN)₂; A is selected from

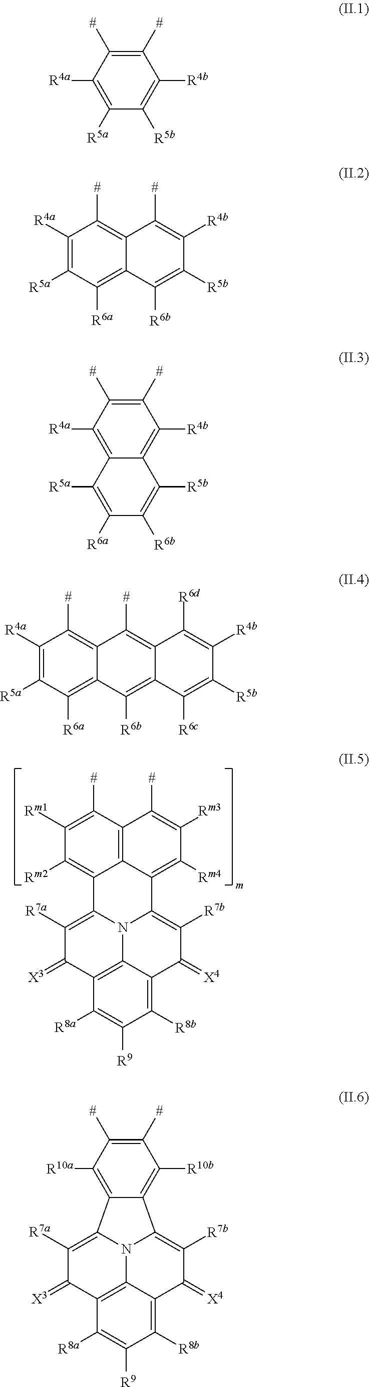

wherein, if present, X³, X⁴ are O or C(CN)₂ and m, R^{4a}, R^{4b}, R^{5a}, R^{5b}, R^{6a}, R^{6b}, R^{6c}, R^{6d}, R^{7a}, R^{7a}, R^{8a}, R^{8b}, R⁹, R^{10a}, R^{10b}, R^{m1}, R^{m2}, R^{m3} and R^{m4} are as defined in the claims and description. The present invention also relates to a method for their preparation and their use as semiconductors, in particular as semiconductors in organic electronics and organic photovoltaics.

### FIELD OF THE INVENTION

The present invention relates to a new class of cycl[3.3.3]azines, a method for their preparation and their use as semiconductors, in particular as semiconductors in organic electronics and organic photovoltaics.

It is expected that, in the future, not only the classical inorganic semiconductors but increasingly also organic semiconductors based on low molecular weight or polymeric materials will be used in many sectors of the electronics industry. In many cases, these organic semiconductors have advantages over the classical inorganic semiconductors, for example better substrate compatibility and better processability of the semiconductor components based on them. They allow processing on flexible substrates and enable their interface orbital energies to be adjusted precisely to the particular application range by the methods of molecular modeling. The significantly reduced costs of such components have brought a renaissance to the field of research of organic electronics.

Organic electronics is concerned principally with the development of new materials and manufacturing processes for the production of electronic components based on organic semiconductor layers. These include in particular organic field-effect transistors (OFETs) and organic electroluminescent devices (hereinafter abbreviated as “EL” devices). Great potential for development is ascribed to organic field-effect transistors, for example in storage elements and integrated optoelectronic devices. An organic electroluminescent device is a self-emission device utilizing the principle that a fluorescent or phosphorescent material emits light by the recombination energy of holes injected from an anode and electrons injected from a cathode when an electric field is applied. EL devices in form of organic light-emitting diodes (OLEDs) are especially of interest as an alternative to cathode ray tubes and liquid-crystal displays for producing flat visual display units. Owing to the very compact design and the intrinsically low power consumption, devices which comprise OLEDs are suitable especially for mobile applications, for example for applications in cell phones, laptops, etc.

Organic photovoltaics is concerned principally with the development of new materials for organic solar cells. A great potential for development is ascribed to materials which have maximum transport widths and high mobilities for light-induced excited states (high exciton diffusion lengths) and are thus advantageously suitable for use as an active material in so-called excitonic solar cells. It is generally possible with solar cells based on such materials to achieve very good quantum yields. There is therefore a great need for organic compounds which are suitable as charge transport materials or exciton transport materials.

Rylenes (or poly(peri-naphthalene)s) and rylene derivatives are a class of chromophores that is characterized by at least two naphthalene units bound to each other in the peri-positions. Naphtalene-tetracarboxylic dianhydrides, rylene-tetracarboxylic dianhydrides and their corresponding diimides have become of outstanding importance as classical colorants as well as active components of electronic and optoelectronic devices. However, it has been found that some of these compounds have application properties that are still worth of improvement. Generally, there is still a need for new colorants that can be easily incorporated into a great variety of polymer compositions. Preferably, these compositions should be processable under the conventional temperatures for thermoplastics, without the color or other optical properties changing significantly during processing. Further, some of the naphthalene and rylene derivatives known from the prior art are still need improvement in terms of their use as a semiconductor material in organic electronics or in organic photovoltaics.

The name cyclazine was originally proposed by V. Boekelheide to denote conjugated unsaturated heterocycles held planar by three covalent bonds to an internal nitrogen atom. The first synthesis of cycl[3.3.3]azine (pyrido-[2,1,6-d,e]quinolizine) was reported by D. Farquhar and D. Leaver in Chem. Comm. 24 (1969). An overview of the synthesis and characterization of cyclazines and related N-bridged annulenes is given by D. Leaver in Pure & Appl. Chem., Vol. 58, No. 1, pp. 143-152 (1986).

H. Z. Alkhathlan, M. A. Al-Jaradah, K. A. Al-Farhan and A. A. Mousa. describe in Phosphorous, Sulfur and Silicon, 179: 378-388, 2004, the preparation of N-heteryliminophosphoranes starting from 2-acetylaniline and phthalic anhydride or 3-nitrophthalic anhydride, 4-nitrophthalic anhydride or 3, 4,5,6-tetrachlorophthalic anhydride. Merely the reaction of 3-nitrophthalic anhydride and 3,4,5,6-tetrachlorophthalic anhydride, respectively, leads to the formation of an isoindolo[2,3-a]quinoline-5,11-dione compound, namely 7-nitroisoindolo[2,3-a]-quinoline-5,11-dione and 7,8,9,10-tetrachloroisoindolo[2,3-a]quinoline-5,11-dione, respectively, as byproduct. No mention is made to any use of 7-nitroisoindolo[2,3-a]quinoline-5,11-dione or 7,8,9,10-tetrachloroisoindolo[2,3-a]quinoline-5,11-dione.

It is already known to employ nitrogen containing unsaturated heterocycles as semiconductors in organic electronics and organic photovoltaics.

WO 2007/031165 A2 describes compounds of the formulae (1) and (2)

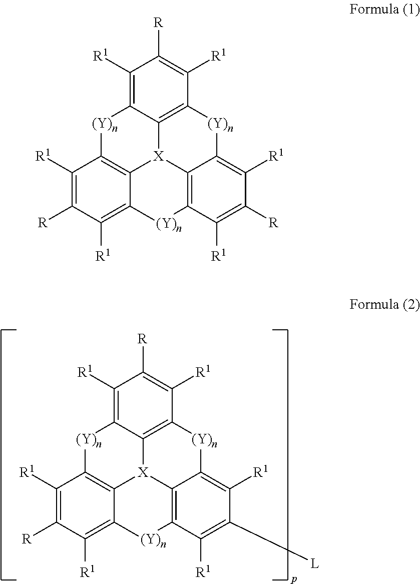

where p is 2 to 6 depending on the valence of the L group, and the use of those compounds in electroluminescent devices, such as OLED and OFET. The concrete compounds contain only one (hetero)triangulene moiety as central core.

Kerstin Schmoltner, Florian Schlütter, Milan Kivala, Martin Baumgarten, Stefanie Winkler, Roman Trattnig, Norbert Koch, Andreas Klug, Emil J. W. List and Klaus Müllen describe in Polym. Chem., 2013, 4, 5337 a heterotriangulene polymer for air-stable organic field-effect transistors.

DE 10 2010 014 933 A1 describes heteroaromatic compounds of the general formula (A) and their use in organic electronics

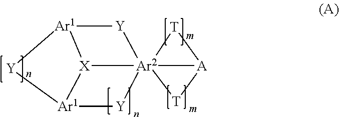

wherein

### X is N, P, or P═O;

Y is, identically or differently on each occurrence, C(R¹)₂, C═O, C═NR¹, O, S, SO, SO₂, PR¹, POR¹, NAr, NR¹, or a single bond;
T is, identically or differently on each occurrence C(R¹)₂, C═O, C═NR¹, O, S, SO, SO₂, PR¹, POR¹, NAr, NR¹, or a single bond;
A is Ar³ or X(Ar⁴)₂, wherein the bond to a group T starts from the aromatic or heteroaromatic ring of the group Ar³ or Ar⁴, and the two groups Ar⁴ of a group X(Ar⁴)₂ are optionally connected to one another via a group T;
Ar, Ar¹, Ar², Ar³, and Ar⁴ are, identically or differently on each occurrence, an aryl or heteroaryl group having 5 to 30 aromatic ring atoms optionally substituted by one or more radicals R².

WO 2012163471 relates to metal complexes of the general formula

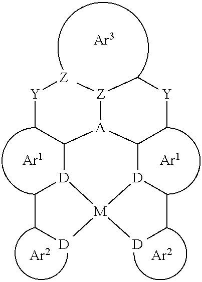

wherein
M is a transition metal;
A is N, P, B, C⁻ or CR;
Y can have in each case the same or different meanings and is CR₂, NR, O, S or a single bond;
Z can have in each case the same or different meanings and is C or N;
Ar¹, Ar² and Ar³ are aryl or heteroaryl group;
   and also to electronic devices, more particularly organic electroluminescent devices, which comprise these metal complexes.

The post-published Angew. Chem. Int. Ed. 2015, 54, 2285-2289 describes the preparation of 2-(tert-butyl)naphtha[1′,8′:7,8,9]quinolizino[3,4,5,6-ija]quinoline-4,13-dione by treating naphthalene monoanhydride with 4-tert-butyl-2,6-diacetyl aniline. Treatment of 2-(tert-butyl)naphtha[1′,8′:7,8,9]quinolizino[3,4,5,6-ija]quinoline-4,13-dione with malodinitrile in the presence of glacial acetic acid and acetic anhydride yields 2,2′-(2-(tert-butyl)naphtha[1′,8′:7,8,9]quinolizino[3,4,5,6-ija]quinoline-4,13-diylidene)dimalononitrile. Described is also the preparation of 5,6,16,17-tetra-tert-octyl-phenoxy-peryleno[3′,4′:7,8,9;9′,10′:7,8,9]diquinolizino[3,4,5,6-ija]quinoline-2,9,13,20-tetrone. The compounds may be used as colorants or as new materials for organic electronics.

It has now been found that, surprisingly, that core-expanded cycl[3.3.3]azines substituted with double bound oxygen or malodinitrile groups are particularly advantageous as semiconductor materials in organic electronics and organic photovoltaics. It was further found that those core-expanded cycl[3.3.3]azines are new chromophores having advantageous application properties as colorants. In particular they have at least one of the following properties:
high photostability,
high thermal stability,
high light fastness,
high molar extinction coefficient,
suitability for security printing.

### SUMMARY OF THE INVENTION

According to a first aspect of the present invention there is provided a compound of the general formula I

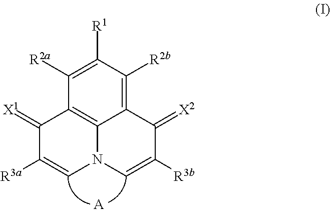

wherein
X¹ and X² are independently of one another selected from O and C(CN)₂;
R¹, R^{2a}, R^{2b}, R^{3a} and R^{3b} are independently of one another selected from hydrogen, F, Cl, Br, I, CN, hydroxy, mercapto, nitro, cyanato, thiocyanato, formyl, acyl, carboxy, carboxylate, alkylcarbonyloxy, carbamoyl, alkylaminocarbonyl, dialkylaminocarbonyl, sulfo, sulfonate, sulfoamino, sulfamoyl, alkylsulfonyl, arylsulfonyl, amidino, NE¹E², where E¹ and E² are each independently selected from hydrogen, alkyl, cycloalkyl, heterocycloalkyl, aryl or hetaryl,
   in each case unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, heterocycloalkyl, heterocycloalkoxy, heterocycloalkylthio, (monoheterocycloalkyl)amino, (diheterocycloalkyl)amino, aryl, aryloxy, arylthio, (monoaryl)amino, (diaryl)amino, hetaryl, hetaryloxy, hetarylthio, (monohetaryl)amino and (dihetaryl)amino;
A is selected from the groups of the general formulae (II.1), (II.2), (II.3), (II.4), (II.5) and (II.6)

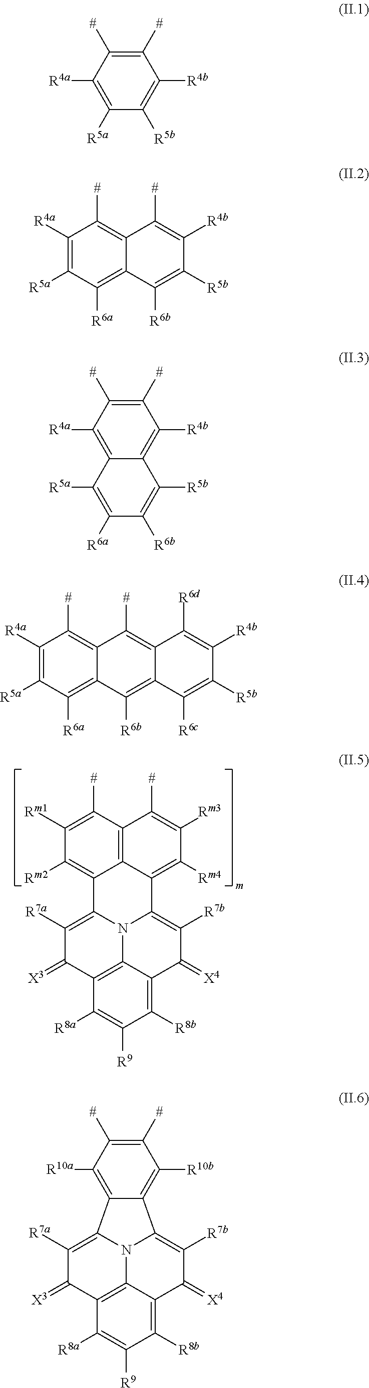

in which
# in each case represents the bonding site to the cyclazine skeleton;
   in the formulae (II.1), (II.2), (II.3), (II.4)
R^{4a}, R^{4b}, R^{5a}, R^{5b}, and, if present, R^{6a}, R^{6b}, R^{6c} and R^{6d} are independently of one another selected from hydrogen, F, Cl, Br, I, CN, hydroxy, mercapto, nitro, cyanato, thiocyanato, formyl, acyl, carboxy, carboxylate, alkylcarbonyloxy, carbamoyl, alkylaminocarbonyl, dialkylaminocarbonyl, sulfo, sulfonate, sulfoamino, sulfamoyl, alkylsulfonyl, arylsulfonyl, amidino, NE¹E², where E¹ and E² are each independently selected from hydrogen, alkyl, cycloalkyl, heterocycloalkyl, aryl or hetaryl,
   in each case unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, heterocycloalkyl, heterocycloalkoxy, heterocycloalkylthio, (monoheterocycloalkyl)amino, (diheterocycloalkyl)amino, aryl, aryloxy, arylthio, (monoaryl)amino, (diaryl)amino, hetaryl, hetaryloxy, hetarylthio, (monohetaryl)amino and (dihetaryl)amino,
m in the formula (II.5) is 1, 2, 3 or 4;
   in the formulae (II.5) and (II.6)
X³ and X⁴ are independently of one another selected from O and C(CN)₂;
R^{7a}, R^{7b}, R^{8a}, R^{8b}, R⁹ and, if present, each R^{m1}, R^{m2}, R^{m3} and R^{m4} are independently of one another selected from hydrogen, F, Cl, Br, I, CN, hydroxy, mercapto, nitro, cyanato, thiocyanato, formyl, acyl, carboxy, carboxylate, alkylcarbonyloxy, carbamoyl, alkylaminocarbonyl, dialkylaminocarbonyl, sulfo, sulfonate, sulfoamino, sulfamoyl, alkylsulfonyl, arylsulfonyl, amidino, NE¹E², where E¹ and E² are each independently selected from hydrogen, alkyl, cycloalkyl, heterocycloalkyl, aryl or hetaryl,
   in each case unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, heterocycloalkyl, heterocycloalkoxy, heterocycloalkylthio, (monoheterocycloalkyl)amino, (diheterocycloalkyl)amino, aryl, aryloxy, arylthio, (monoaryl)amino, (diaryl)amino, hetaryl, hetaryloxy, hetarylthio, (monohetaryl)amino and (dihetaryl)amino;
R^{10a} and R^{10b}, if present, are independently of one another selected from hydrogen, F, Cl, Br, I, CN, hydroxy, mercapto, nitro, cyanato, thiocyanato, formyl, acyl, carboxy, carboxylate, alkylcarbonyloxy, carbamoyl, alkylaminocarbonyl, dialkylaminocarbonyl, sulfo, sulfonate, sulfoamino, sulfamoyl, alkylsulfonyl, arylsulfonyl, amidino, NE¹E², where E¹ and E² are each independently selected from hydrogen, alkyl, cycloalkyl, heterocycloalkyl, aryl and hetaryl,
   in each case unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, heterocycloalkyl, heterocycloalkoxy, heterocycloalkylthio, (monoheterocycloalkyl)amino, (diheterocycloalkyl)amino, aryl, aryloxy, arylthio, (monoaryl)amino, (diaryl)amino, hetaryl, hetaryloxy, hetarylthio, (monohetaryl)amino and (dihetaryl)amino.

In a preferred embodiment the compounds of the general formula I are selected from compounds of the formulae (I.A) and (I.B)

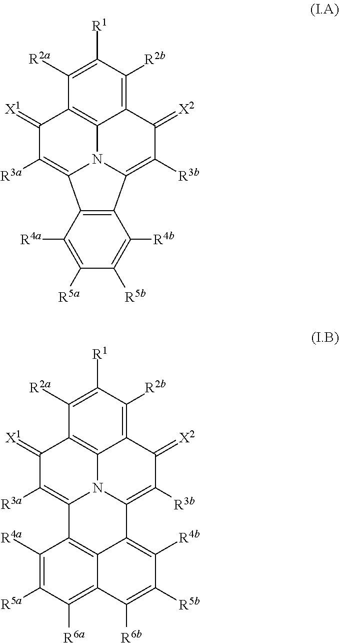

wherein
X¹, X², R¹, R^{2a}, R^{2b}, R^{3a} and R^{3b} are defined as above-mentioned,
R^{4a}, R^{4b}, R^{5a}, R^{5b}, and, if present, R^{6a} and R^{6b} are independently of one another selected from hydrogen, F, Cl, Br, I, CN, hydroxy, mercapto, nitro, cyanato, thiocyanato, formyl, acyl, carboxy, carboxylate, alkylcarbonyloxy, carbamoyl, alkylaminocarbonyl, dialkylaminocarbonyl, sulfo, sulfonate, sulfoamino, sulfamoyl, alkylsulfonyl, arylsulfonyl, amidino, NE¹E², where E¹ and E² are each independently selected from hydrogen, alkyl, cycloalkyl, heterocycloalkyl, aryl or hetaryl,
   in each case unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, heterocycloalkyl, heterocycloalkoxy, heterocycloalkylthio, (monoheterocycloalkyl)amino, (diheterocycloalkyl)amino, aryl, aryloxy, arylthio, (monoaryl)amino, (diaryl)amino, hetaryl, hetaryloxy, hetarylthio, (monohetaryl)amino and (dihetaryl)amino.

In a further preferred embodiment the compounds of the general formula I are selected from compounds of the formula (I.C)

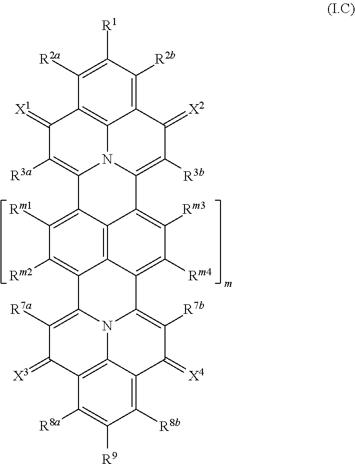

wherein
m is 1, 2, 3 or 4,
X¹, X², X³ and X⁴ are independently of one another selected from O and C(CN)₂,
R¹, R^{2a}, R^{2b}, R^{3a}, R^{3b}, R^{7a}, R^{7b}, R^{8a}, R^{8b}, R⁹ and each R^{m1}, R^{m2}, R^{m3} and R^{m4} are independently of one another selected from hydrogen, F, Cl, Br, I, CN, hydroxy, mercapto, nitro, cyanato, thiocyanato, formyl, acyl, carboxy, carboxylate, alkylcarbonyloxy, carbamoyl, alkylaminocarbonyl, dialkylaminocarbonyl, sulfo, sulfonate, sulfoamino, sulfamoyl, alkylsulfonyl, arylsulfonyl, amidino, NE¹E², where E¹ and E² are each independently selected from hydrogen, alkyl, cycloalkyl, heterocycloalkyl, aryl or hetaryl,
   in each case unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, heterocycloalkyl, heterocycloalkoxy, heterocycloalkylthio, (monoheterocycloalkyl)amino, (diheterocycloalkyl)amino, aryl, aryloxy, arylthio, (monoaryl)amino, (diaryl)amino, hetaryl, hetaryloxy, hetarylthio, (monohetaryl)amino and (dihetaryl)amino.

A special embodiment are compounds of the formula (I.Ca)

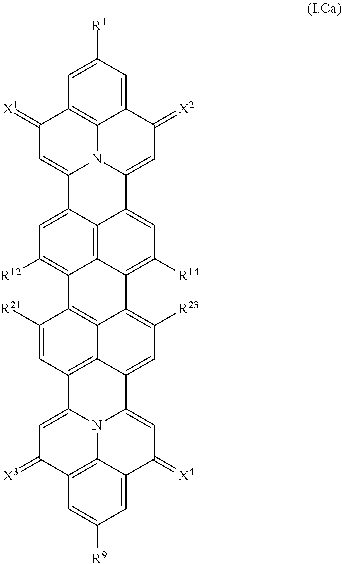

wherein
X¹, X², X³ and X⁴ are all O or all C(CN)₂,
R¹ and R⁹ are independently of one another selected from hydrogen, F, Cl, Br, I, CN, hydroxy, mercapto, nitro, cyanato, thiocyanato, formyl, acyl, carboxy, carboxylate, alkylcarbonyloxy, carbamoyl, alkylaminocarbonyl, dialkylaminocarbonyl, sulfo, sulfonate, sulfoamino, sulfamoyl, alkylsulfonyl, arylsulfonyl, amidino, NE¹E², where E¹ and E² are each independently selected from hydrogen, alkyl, cycloalkyl, heterocycloalkyl, aryl or hetaryl,
   in each case unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, heterocycloalkyl, heterocycloalkoxy, heterocycloalkylthio, (monoheterocycloalkyl)amino, (diheterocycloalkyl)amino, aryl, aryloxy, arylthio, (monoaryl)amino, (diaryl)amino, hetaryl, hetaryloxy, hetarylthio, (monohetaryl)amino and (dihetaryl)amino, and
R¹⁴ and R²¹ are both hydrogen and R¹² and R²³ have the same meaning and are selected from F, Cl, Br, I, CN and in each case unsubstituted or substituted aryl, aryloxy and arylthio,
or R²¹ and R²³ are both hydrogen and R¹² and R¹⁴ have the same meaning and are selected from F, Cl, Br, I, CN and in each case unsubstituted or substituted aryl, aryloxy and arylthio,
or R¹², R¹⁴, R²¹ and R²³ have the same meaning and are selected from hydrogen, F, Cl, Br, I, CN and in each case unsubstituted or substituted aryl, aryloxy and arylthio.

In a further preferred embodiment the compounds of the general formula I are selected from compounds of the formula (I.D)

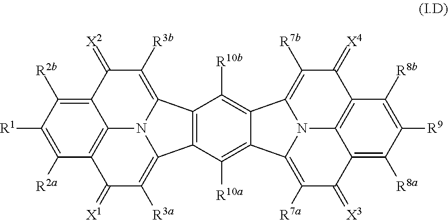

wherein
X¹, X², X³ and X⁴ are independently of one another selected from O and C(CN)₂;
R¹, R^{2a}, R^{2b}, R^{3a}, R^{3b}, R^{7a}, R^{7b}, R^{8a}, R^{8b}, R⁹, R^{10a} and R^{10b} are independently of one another selected from hydrogen, F, Cl, Br, I, CN, hydroxy, mercapto, nitro, cyanato, thiocyanato, formyl, acyl, carboxy, carboxylate, alkylcarbonyloxy, carbamoyl, alkylaminocarbonyl, dialkylaminocarbonyl, sulfo, sulfonate, sulfoamino, sulfamoyl, alkylsulfonyl, arylsulfonyl, amidino, NE¹E², where E¹ and E² are each independently selected from hydrogen, alkyl, cycloalkyl, heterocycloalkyl, aryl and hetaryl,
   in each case unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, heterocycloalkyl, heterocycloalkoxy, heterocycloalkylthio, (monoheterocycloalkyl)amino, (diheterocycloalkyl)amino, aryl, aryloxy, arylthio, (monoaryl)amino, (diaryl)amino, hetaryl, hetaryloxy, hetarylthio, (monohetaryl)amino and (dihetaryl)amino.

According to a further aspect of the present invention there is provided a process for the preparation of a compound of the formula I.A, a process for the preparation of a compound of the formula I.B and a process for the preparation of a compound of the formula I.C. According to a further aspect of the present invention there is provided a process for the preparation of a compound of the formula I.D.

According to a further aspect of the present invention there is provided an organic field-effect transistor comprising a substrate having at least one gate structure, a source electrode and a drain electrode and at least one compound of the formula I as defined above and in the following as a semiconductor material.

The compounds of the formula (I) can be in principle used as n-type semiconductors or as p-type semiconductors. If a compound of the formula (I) acts as n-type semiconductor or as p-type semiconductors depends inter alia on the employed gate dielectric. Gate dielectrics are usually employed in the form of a self-assembled monolayer (SAM) of suitable compounds, e.g. silanes with more or less electronegative substituents, alkyl phosphonic acid, fluoroalkyl phosphonic acid, etc. By choosing a certain SAM gate dielectric or a certain mixture of different SAM gate dielectrics, it is possible to control the properties of the semiconductor material. In electronic devices that employ a combination of two different semiconductors, e.g. organic solar cells, it depends on the corresponding semiconductor material if a compound of the formula (I) acts as n-type semiconductor or as p-type semiconductor.

According to a further aspect of the present invention there is provided a substrate comprising a plurality of organic field-effect transistors, at least some of the field-effect transistors comprising at least one compound of the formula I as defined above and in the following.

According to a further aspect of the present invention there is provided a semiconductor unit comprising at least one substrate comprising a plurality of organic field-effect transistors, at least some of the field-effect transistors comprising at least one compound of the formula I as defined above and in the following.

According to a further aspect of the present invention there is provided an electroluminescent arrangement comprising an upper electrode, a lower electrode, wherein at least one of said electrodes is transparent, an electroluminescent layer and optionally an auxiliary layer, wherein the electroluminescent arrangement comprises at least one compound of the formula I as defined above and in the following.

In a preferred embodiment, the electroluminescent arrangement is in form of an organic light-emitting diode (OLED).

According to a further aspect of the present invention there is provided an organic solar cell comprising at least one compound of the formula (I) as defined above and in the following.

According to a further aspect of the present invention there is provided the use of a compound of the general formula I, as defined above and in the following, as a semiconductor material.

In a preferred embodiment, the compound of the general formula I are used as a semiconductor material in organic electronics or in organic photovoltaics.

According to a further aspect of the present invention there is provided a composition comprising at least one compound of the formula (I) as defined above and in the following and at least polymer, preferably at least one thermoplastic polymer.

According to a further aspect of the present invention there is provided the use of a compound of the general formula I, as defined above and in the following
as fluorescent colorants, preferably as fluorescent colorants in a display based on fluorescence conversion,
for data storage,
as a UV absorber,
for optical labels,
as a fluorescent label for biomolecules,
in the laser welding of polymer materials,
in inks, preferably in ink jet inks and printing inks,
in surface coatings preferably as or in the colored layer of a coating composition, in particular in a coating composition for the automotive industry, and
for coloring polymer compositions.

### DETAILED DESCRIPTION OF THE INVENTION

The novel compounds of the formula I wherein A is a group of the formula (II.5) and the corresponding compounds of the formula (I.C) are characterized by an aromatic core formed by a naphthalene or a rylene system (perylene, terrylene or quaterrylene) which is core-extended at the peri-positions by an iso-juline-1,7-dione unit with two aminoenone moieties fully embracing the nitrogen atom:

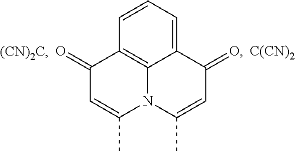

In the compounds of the formula I wherein A is a group of the formula (II.5) and in the corresponding compounds of the formula (I.C) m is the number of naphthalene units which for m=2, 3 or 4 are bonded in the peri-position and form the basic skeleton of the rylene compounds. In the individual R^{m1} to R^{m4} radicals, m is the particular naphthalene group of the rylene skeleton to which the radicals are bonded. R^{m1} to R^{m4} radicals which are bonded to different naphthalene groups may each have the same or different definitions. Accordingly, the compounds of the formula I wherein A is a group of the formula (II.5) and the corresponding compounds of the formula (I.C) may have the following formulae:

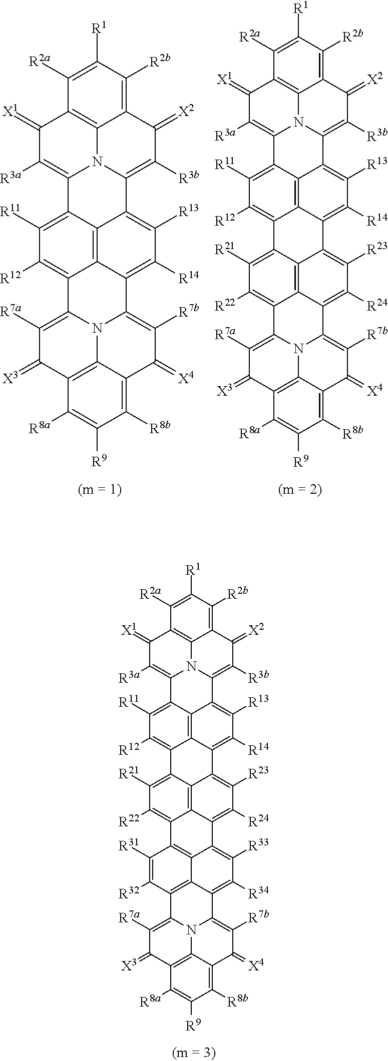

The higher homologues (m=4) are formed analogously.

The expression “halogen” denotes in each case fluorine, bromine, chlorine or iodine, particularly chlorine, bromine or iodine.

In the context of the invention, the expression “unsubstituted or substituted alkyl, cycloalkyl, heterocycloalkyl, aryl and hetaryl” represents unsubstituted or substituted alkyl, unsubstituted or substituted cycloalkyl, unsubstituted or substituted heterocycloalkyl, unsubstituted or substituted aryl and unsubstituted or substituted hetaryl.

In the context of the invention, the expression “unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, heterocycloalkyl, heterocycloalkoxy, heterocycloalkylthio, (monoheterocycloalkyl)amino, (diheterocycloalkyl)amino, aryl, aryloxy, arylthio, (monoaryl)amino, (diaryl)amino, hetaryl, hetaryloxy, hetarylthio, (monohetaryl)amino and (dihetaryl)amino” represents unsubstituted or substituted alkyl, unsubstituted or substituted alkoxy, unsubstituted or substituted alkylthio, unsubstituted or substituted (monoalkyl)amino, unsubstituted or substituted (dialkyl)amino, unsubstituted or substituted cycloalkyl, unsubstituted or substituted cycloalkoxy, unsubstituted or substituted cycloalkylthio, unsubstituted or substituted (monocycloalkyl)amino, unsubstituted or substituted (dicycloalkyl)amino, unsubstituted or substituted heterocycloalkyl, unsubstituted or substituted heterocycloalkoxy, unsubstituted or substituted heterocycloalkylthio, unsubstituted or substituted (monoheterocycloalkyl)amino, unsubstituted or substituted (diheterocycloalkyl)amino, unsubstituted or substituted aryl, unsubstituted or substituted aryloxy, unsubstituted or substituted arylthio, unsubstituted or substituted (monoaryl)amino, unsubstituted or substituted (diaryl)amino, unsubstituted or substituted hetaryl, unsubstituted or substituted hetaryloxy, unsubstituted or substituted hetarylthio, unsubstituted or substituted (monohetaryl)amino and unsubstituted or substituted (dihetaryl)amino.

In the context of the present invention, the expression “alkyl” comprises straight-chain or branched alkyl groups. Alkyl is preferably C₁-C₃₀-alkyl, more preferably C₁-C₂₀-alkyl and most preferably C₁-C₁₂-alkyl. Examples of alkyl groups are especially methyl, ethyl, n-propyl, isopropyl, n-butyl, isobutyl, sec-butyl, tert-butyl, n-pentyl, 1-methylbutyl, 1-ethylpropyl, neo-pentyl, n-hexyl, 1-methylpentyl, 2-methylpentyl, 1-ethylbutyl, 2-ethylbutyl, n-heptyl, 1-methylhexyl, 2-methylhexyl, 1-ethylpentyl, 1-propylbutyl, 2-ethylpentyl, n-octyl, 1-methylheptyl, 2-methylheptyl, 1-ethylhexyl, 2-ethylhexyl, 1-propylpentyl, 2-propylpentyl, n-nonyl, 1-methyloctyl, 2-methyloctyl, 1-ethylheptyl, 2-ethylheptyl, 1-propylhexyl, 2-propylhexyl, 1-butylpentyl, n-decyl, 2-methyldecyl, 1-methylnonyl, 2-methylnonyl, 1-ethyloctyl, 2-ethyloctyl, 1-propylheptyl, 2-propylheptyl, 1-butylhexyl, 2-butylhexyl, n-undecyl, 2-ethylnonyl, 1-propyloctyl, 2-propyloctyl, 1-butylheptyl, 2-butylheptyl, 1-pentylhexyl, n-dodecyl, 2-ethyldecyl, 2-propylnonyl, 1-butyloctyl, 2-butyloctyl, 1-pentylheptyl, 2-pentylheptyl, 2-propyldecyl, n-tridecyl, 1-pentyloctyl, 2-pentyloctyl, 1-hexylheptyl, 2-butylnonyl, n-tetradecyl, 1-hexyloctyl, 2-hexyloctyl, 2-pentylnonyl, 2-hexylnonyl, 2-pentyldecyl, 2-butyldecyl,n-hexadecyl, 1-heptyloctyl, 2-heptylnonyl, 2-hexyldecyl, 2-heptyldecyl, n-octadecyl, 2-octyldecyl, n-eicosyl, 2-nonylundecyl, 2-octylundecyl, 2-heptylundecyl, 2-hexylundecyl, 2-pentylundecyl, 2-butylundecyl, 2-propylundecyl, 2-ethylundecyl, 2-methylundecyl, 2-decyldodecyl, 2-nonyldodecyl, 2-octyldodecyl, 2-heptyldodecyl, 2-hexyldodecyl, 2-pentyldodecyl, 2-butyldodecyl, 2-propyldodecyl, 2-ethyldodecyl, 2-methyldodecyl, 2-undecyltridecyl, 2-decyltridecyl, 2-nonyltridecyl, 2-octyltridecyl, 2-heptyltridecyl, 2-hexyltridecyl, 2-pentyltridecyl, 2-butyltridecyl, 2-propyltridecyl, 2-ethyltridecyl, 2-methyltridecyl, 2-undecyltetradecyl, 2-decyltetradecyl, 2-nonyltetradecyl, 2-octyltetradecyl, 2-hetyltetradecyl, 2-hexyltetradecyl, 2-pentyltetradecyl, 2-butyltetradecyl, 2-propyltetradecyl, 2-ethyltetradecyl, 2-methyltetradecyl, 2-tetradecylhexadecyl, 2-tridecylhexadecyl, 2-dodecylhexadecyl, 2-undecylhexadecyl, 2-decylhexadecyl, 2-nonylhexadecyl, 2-octylhexadecyl, 2-heptylhexadecyl, 2-hexylhexadecyl, 2-pentylhexadecyl, 2-butylhexadecyl, 2-propylhexadecyl, 2-ethylhexadecyl, 2-methylhexadecyl, 2-dodecyloctadecyl, 2-undecyloctadecyl, 2-decyloctadecyl, 2-nonyloctadecyl, 2-octyloctadecyl, 2-heptyloctadecyl, 2-hexyloctadecyl, 2-pentyloctadecyl, 2-butyloctadecyl, 2-propyloctadecyl, 2-ethyloctadecyl, 2-methyloctadecyl, 2-decyleicosanyl, 2-nonyleicosanyl, 2-octyleicosanyl, 2-heptyleicosanyl, 2-hexyleicosanyl, 2-pentyleicosanyl, 2-butyleicosanyl, 2-propyleicosanyl, 2-ethyleicosanyl, 2-methyleicosanyl, 2-octadecyldocosanyl, 2-heptadecyldocosanyl, 2-hexadecyldocosanyl, 2-pentadecyldocosanyl, 2-tetradecyldocosanyl, 2-tridecyldocosanyl, 2-undecyldocosanyl, 2-decyldocosanyl, 2-nonyldocosanyl, 2-octyldocosanyl, 2-heptyldocosanyl, 2-hexyldocosanyl, 2-pentyldocosanyl, 2-butyldocosanyl, 2-propyldocosanyl, 2-ethyldocosanyl, 2-methyldocosanyl, 2-docosanyltetracosanyl, 2-hexadecyltetracosanyl, 2-pentadecyltetracosanyl, 2-pentadecyltetracosanyl, 2-tetradecyltetracosanyl, 2-tridecyltetracosanyl, 2-dodecyltetracosanyl, 2-undecyltetracosanyl, 2-decyltetracosanyl, 2-nonyltetracosanyl, 2-octyltetracosanyl, 2-heptyltetracosanyl, 2-hexyltetracosanyl, 2-pentyltetracosanyl, 2-butyltetracosanyl, 2-propyltetracosanyl, 2-ethyltetracosanyl, 2-methyltetracosanyl, 2-dodecyloctacosanyl, 2-undecyloctacosanyl, 2-decyloctacosanyl, 2-nonyloctacosanyl, 2-octyloctacosanyl, 2-heptyloctacosanyl, 2-hexyloctacosanyl, 2-pentyloctacosanyl, 2-butyloctacosanyl, 2-propyloctacosanyl, 2-ethyloctacosanyl and 2-methyloctacosanyl.

The expression alkyl also comprises alkyl radicals whose carbon chains may be interrupted by one or more nonadjacent groups which are selected from —O—, —S—, —NR^{a}—, —C(═O)—, —S(═O)— and/or —S(═O)₂—. R^{a} is preferably hydrogen, alkyl, cycloalkyl, heterocycloalkyl, aryl or hetaryl.

Examples of alkyl groups whose carbon chains are interrupted by one or more nonadjacent groups are especially 2-methoxyethyl, 2-ethoxyethyl, 2-propoxyethyl, 2-isopropoxyethyl, 2-butoxyethyl, 2- and 3-methoxypropyl, 2- and 3-ethoxypropyl, 2- and 3-propoxypropyl, 2- and 3-butoxypropyl, 2- and 4-methoxybutyl, 2- and 4-ethoxybutyl, 2- and 4-propoxybutyl, 3,6-dioxaheptyl, 3,6-dioxaoctyl, 4,8-dioxanonyl, 3,7-dioxaoctyl, 3,7-dioxanonyl, 4,7-dioxaoctyl, 4,7-dioxanonyl, 2- and 4-butoxybutyl, 4,8-dioxadecyl, 3,6,9-trioxadecyl, 3,6,9-trioxaundecyl, 3,6,9-trioxadodecyl, 3,6,9,12-tetraoxatridecyl and 3,6,9,12-tetraoxatetradecyl; 2-methylthioethyl, 2-ethylthioethyl, 2-propylthioethyl, 2-isopropylthioethyl, 2-butylthioethyl, 2- and 3-methylthiopropyl, 2- and 3-ethylthiopropyl, 2- and 3-propylthiopropyl, 2- and 3-butylthiopropyl, 2- and 4-methylthiobutyl, 2- and 4-ethylthiobutyl, 2- and 4-propylthiobutyl, 3,6-dithiaheptyl, 3,6-dithiaoctyl, 4,8-dithianonyl, 3,7-dithiaoctyl, 3,7-dithianonyl, 2- and 4-butylthiobutyl, 4,8-dithiadecyl, 3,6,9-trithiadecyl, 3,6,9-trithiaundecyl, 3,6,9-trithiadodecyl, 3,6,9,12-tetrathiatridecyl and 3,6,9,12-tetrathiatetradecyl; 2-monomethyl- and 2-monoethylaminoethyl, 2-dimethylaminoethyl, 2- and 3-dimethylaminopropyl, 3-monoisopropylaminopropyl, 2- and 4-monopropylaminobutyl, 2- and 4-dimethylaminobutyl, 6-methyl-3,6-diazaheptyl, 3,6-dimethyl-3,6-diazaheptyl, 3,6-diazaoctyl, 3,6-dimethyl-3,6-diazaoctyl, 9-methyl-3,6,9-triazadecyl, 3,6,9-trimethyl-3,6,9-triazadecyl, 3,6,9-triazaundecyl, 3,6,9-trimethyl-3,6,9-triazaundecyl, 12-methyl-3,6,9,12-tetraazatridecyl and 3,6,9,12-tetramethyl-3,6,9,12-tetraazatridecyl; (1-ethylethylidene)aminoethylene, (1-ethylethylidene)-aminopropylene, (1-ethylethylidene)aminobutylene, (1-ethylethylidene)aminodecylene and (1-ethylethylidene)aminododecylene; propan-2-on-1-yl, butan-3-on-1-yl, butan-3-on-2-yl and 2-ethylpentan-3-on-1-yl; 2-methylsulfoxidoethyl, 2-ethylsulfoxidoethyl, 2-propylsulfoxidoethyl, 2-isopropylsulf-oxidoethyl, 2-butylsulfoxidoethyl, 2- and 3-methylsulfoxidopropyl, 2- and 3-ethylsulf-oxidopropyl, 2- and 3-propylsulfoxidopropyl, 2- and 3-butylsulfoxidopropyl, 2- and 4-methylsulfoxidobutyl, 2- and 4-ethylsulfoxidobutyl, 2- and 4-propylsulfoxidobutyl and 4-butylsulfoxidobutyl; 2-methylsulfonylethyl, 2-ethylsulfonylethyl, 2-propylsulfonylethyl, 2-isopropylsulfonyl-ethyl, 2-butylsulfonylethyl, 2- and 3-methylsulfonylpropyl, 2- and 3-ethylsulfonylpropyl, 2- and 3-propylsulfonylpropyl, 2- and 3-butylsulfonylpropyl, 2- and 4-methylsulfonyl-butyl, 2- and 4-ethylsulfonylbutyl, 2- and 4-propylsulfonylbutyl and 4-butylsulfonylbutyl.

Substituted alkyl groups may, depending on the length of the alkyl chain, have one or more (e.g. 1, 2, 3, 4, 5 or more than 5) substituents. These are preferably each independently selected from cycloalkyl, heterocycloalkyl, aryl, hetaryl, fluorine, chlorine, bromine, hydroxyl, mercapto, cyano, nitro, nitroso, formyl, acyl, COOH, carboxylate, alkylcarbonyloxy, carbamoyl, SO₃H, sulfonate, sulfamino, sulfamide, amidino, NE⁵E⁶ where E⁵ and E⁶ are each independently hydrogen, alkyl, cycloalkyl, heterocycloalkyl, aryl or hetaryl. Cycloalkyl, heterocycloalkyl, aryl and hetaryl substituents of the alkyl groups may in turn be unsubstituted or substituted; suitable substituents are the substituents mentioned below for these groups. Special embodiments of substituted alkyl groups are perfluoro-C₁-C₃₀-alkyl, 1H,1H-perfluoro-C₂-C₃₀-alkyl and 1H,1H,2H,2H-perfluoro-C₃-C₃₀-alkyl. Examples for those fluorinated alkyl groups are mentioned in the following.

Examples of substituted alkyl groups are especially carboxymethyl, 2-carboxyethyl, 3-carboxypropyl, 4-carboxybutyl, 5-carboxypentyl, 6-carboxyhexyl, 8-carboxyoctyl, 10-carboxydecyl, 12-carboxydodecyl and 14-carboxy-tetradecyl; sulfomethyl, 2-sulfoethyl, 3-sulfopropyl, 4-sulfobutyl, 5-sulfopentyl, 6-sulfohexyl, 8-sulfooctyl, 10-sulfodecyl, 12-sulfododecyl and 14-sulfotetradecyl; 2-hydroxyethyl, 2- and 3-hydroxypropyl, 1-hydroxyprop-2-yl, 3- and 4-hydroxybutyl, 1-hydroxybut-2-yl and 8-hydroxy-4-oxaoctyl; 2-cyanoethyl, 3-cyanopropyl, 3- and 4-cyanobutyl, 2-methyl-3-ethyl-3-cyanopropyl, 7-cyano-7-ethylheptyl and 4,7-dimethyl-7-cyanoheptyl; 2-chloroethyl, 2- and 3-chloropropyl, 2-, 3- and 4-chlorobutyl, 2-bromoethyl, 2- and 3-bromopropyl and 2-, 3- and 4-bromobutyl; 2-nitroethyl, 2- and 3-nitropropyl and 2-, 3- and 4-nitrobutyl.

Carboxylate and sulfonate respectively represent a derivative of a carboxylic acid function and a sulfonic acid function, especially a metal carboxylate or sulfonate, a carboxylic ester or sulfonic ester function or a carboxamide or sulfonamide function. Such derivatives include, for example, esters with C₁-C₄-alkanols, such as methanol, ethanol, n-propanol, isopropanol, n-butanol, sec-butanol and tert-butanol.

The above remarks regarding alkyl also apply to the alkyl moiety in alkoxy, alkylthio (=alkylsulfanyl), monoalkylamino and dialkylamino.

Examples of alkoxy groups are especially methoxy, ethoxy, propoxy, isopropoxy, butoxy, isobutoxy, sec-butoxy, tert-butoxy, pentoxy, isopentoxy, neopentoxy, tert-pentoxy and hexoxy.

Examples of alkylthio groups are especially methylthio, ethylthio, propylthio, isopropylthio, butylthio, isobutylthio, sec-butylthio, tert-butylthio, pentylthio, isopentylthio, neopentylthio, tert-pentylthio and hexylthio.

Examples of monoalkylamino groups and dialkylamino groups are especially methylamino, ethylamino, propylamino, isopropylamino, butylamino, isobutylamino, pentylamino, hexylamino, dimethylamino, methylethylamino, diethylamino, dipropylamino, diisopropylamino, dibutylamino, diisobutylamino, dipentylamino, dihexylamino, dicyclopentylamino, dicyclohexylamino, dicycloheptylamino, diphenylamino and dibenzylamino;

Alkylene represents a linear saturated hydrocarbon chain having from 1 to 10 and especially from 1 to 4 carbon atoms, such as ethane-1,2-diyl, propane-1,3-diyl, butane-1,4-diyl, pentane-1,5-diyl or hexane-1,6-diyl.

In the context of the present invention, the term “cycloalkyl” denotes a mono-, bi- or tricyclic hydrocarbon radical having usually from 3 to 20, preferably 3 to 12, more preferably 5 to 12, carbon atoms such as cyclopropyl, cyclobutyl, cyclopentyl, cyclohexyl, cycloheptyl, cyclooctyl, cyclododecyl, cyclopentadecyl, norbornyl, bicyclo[2.2.2]octyl or adamantyl.

Substituted cycloalkyl groups may, depending on the ring size, have one or more (e.g. 1, 2, 3, 4, 5 or more than 5) substituents. These are preferably each independently selected from alkyl, alkoxy, alkylthio, cycloalkyl, heterocycloalkyl, aryl, hetaryl, fluorine, chlorine, bromine, hydroxyl, mercapto, cyano, nitro, nitroso, formyl, acyl, COOH, carboxylate, alkylcarbonyloxy, carbamoyl, SO₃H, sulfonate, sulfamino, sulfamide, amidino, NE⁷E⁸ where E⁷ and E⁸ are each independently hydrogen, alkyl, cycloalkyl, heterocycloalkyl, aryl or hetaryl. In the case of substitution, the cycloalkyl groups preferably bear one or more, for example one, two, three, four or five, C₁-C₆-alkyl groups. Examples of substituted cycloalkyl groups are especially 2- and 3-methyl-cyclopentyl, 2- and 3-ethylcyclopentyl, 2-, 3- and 4-methylcyclohexyl, 2-, 3- and 4-ethylcyclohexyl, 2-, 3- and 4-propylcyclohexyl, 2-, 3- and 4-isopropylcyclohexyl, 2-, 3- and 4-butylcyclohexyl, 2-, 3- and 4-sec.-butylcyclohexyl, 2-, 3- and 4-tert-butylcyclohexyl, 2-, 3- and 4-methylcycloheptyl, 2-, 3- and 4-ethylcycloheptyl, 2-, 3- and 4-propylcycloheptyl, 2-, 3- and 4-isopropylcycloheptyl, 2-, 3- and 4-butylcycloheptyl, 2-, 3- and 4-sec-butylcycloheptyl, 2-, 3- and 4-tert-butylcycloheptyl, 2-, 3-, 4- and 5-methyl-cyclooctyl, 2-, 3-, 4- and 5-ethylcyclooctyl, 2-, 3-, 4- and 5-propylcyclooctyl, 3- and 4-hydroxycyclohexyl, 3- and 4-nitrocyclohexyl and 3- and 4-chlorocyclohexyl.

The above remarks regarding cycloalkyl also apply to the cycloalkyl moiety in cycloalkoxy, cycloalkylthio (=cycloalkylsulfanyl), monocycloalkylamino and dicycloalkylamino.

In the context of the present invention, the term “aryl” refers to mono- or polycyclic aromatic hydrocarbon radicals. Aryl usually is an aromatic radical having 6 to 24 carbon atoms, preferably 6 to 20 carbon atoms, especially 6 to 14 carbon atoms as ring members. Aryl is preferably phenyl, naphthyl, indenyl, fluorenyl, anthracenyl, phenanthrenyl, naphthacenyl, chrysenyl, pyrenyl, coronenyl, perylenyl, etc., and more preferably phenyl or naphthyl.

Substituted aryls may, depending on the number and size of their ring systems, have one or more (e.g. 1, 2, 3, 4, 5 or more than 5) substituents. These are preferably each independently selected from alkyl, alkoxy, alkylthio, cycloalkyl, heterocycloalkyl, aryl, hetaryl, fluorine, chlorine, bromine, hydroxyl, mercapto, cyano, nitro, nitroso, formyl, acyl, COOH, carboxylate, alkylcarbonyloxy, carbamoyl, SO₃H, sulfonate, sulfamino, sulfamide, amidino, NE⁹E¹⁰ where E⁹ and E¹⁰ are each independently hydrogen, alkyl, cycloalkyl, heterocycloalkyl, aryl or hetaryl. The alkyl, alkoxy, alkylamino, alkylthio, cycloalkyl, heterocycloalkyl, aryl and hetaryl substituents on the aryl may in turn be unsubstituted or substituted. Reference is made to the substituents mentioned above for these groups. The substituents on the aryl are preferably selected from alkyl, alkoxy, haloalkyl, haloalkoxy, aryl, fluorine, chlorine, bromine, cyano and nitro. Substituted aryl is more preferably substituted phenyl which generally bears 1, 2, 3, 4 or 5, preferably 1, 2 or 3, substituents.

Substituted aryl is preferably aryl substituted by at least one alkyl group (“alkaryl”, also referred to hereinafter as alkylaryl). Alkaryl groups may, depending on the size of the aromatic ring system, have one or more (e.g. 1, 2, 3, 4, 5, 6, 7, 8, 9 or more than 9) alkyl substituents. The alkyl substituents may be unsubstituted or substituted. In this regard, reference is made to the above statements regarding unsubstituted and substituted alkyl. In a preferred embodiment, the alkaryl groups have exclusively unsubstituted alkyl substituents. Alkaryl is preferably phenyl which bears 1, 2, 3, 4 or 5, preferably 1, 2 or 3, more preferably 1 or 2, alkyl substituents.

Aryl which bears one or more radicals is, for example, 2-, 3- and 4-methylphenyl, 2,4-, 2,5-, 3,5- and 2,6-dimethylphenyl, 2,4,6-trimethylphenyl, 2-, 3- and 4-ethylphenyl, 2,4-, 2,5-, 3,5- and 2,6-diethylphenyl, 2,4,6-triethylphenyl, 2-, 3- and 4-propylphenyl, 2,4-, 2,5-, 3,5- and 2,6-dipropylphenyl, 2,4,6-tripropylphenyl, 2-, 3- and 4-isopropylphenyl, 2,4-, 2,5-, 3,5- and 2,6-diisopropylphenyl, 2,4,6-triisopropylphenyl, 2-, 3- and 4-butylphenyl, 2,4-, 2,5-, 3,5- and 2,6-dibutylphenyl, 2,4,6-tributylphenyl, 2-, 3- and 4-isobutylphenyl, 2,4-, 2,5-, 3,5- and 2,6-diisobutylphenyl, 2,4,6-triisobutylphenyl, 2-, 3- and 4-sec-butylphenyl, 2,4-, 2,5-, 3,5- and 2,6-di-sec-butylphenyl, 2,4,6-tri-sec-butylphenyl, 2-, 3- and 4-tert-butylphenyl, 2,4-, 2,5-, 3,5- and 2,6-di-tert-butylphenyl and 2,4,6-tri-tert-butylphenyl; 2-, 3- and 4-methoxyphenyl, 2,4-, 2,5-, 3,5- and 2,6-dimethoxyphenyl, 2,4,6-trimethoxyphenyl, 2-, 3- and 4-ethoxyphenyl, 2,4-, 2,5-, 3,5- and 2,6-diethoxyphenyl, 2,4,6-triethoxyphenyl, 2-, 3- and 4-propoxyphenyl, 2,4-, 2,5-, 3,5- and 2,6-dipropoxyphenyl, 2-, 3- and 4-isopropoxyphenyl, 2,4-, 2,5-, 3,5- and 2,6-diisopropoxyphenyl and 2-, 3- and 4-butoxyphenyl; 2-, 3- and 4-chlorophenyl, (2-chloro-6-methyl)phenyl, (2-chloro-6-ethyl)phenyl, (4-chloro-6-methyl)phenyl, (4-chloro-6-ethyl)phenyl.

The above remarks regarding aryl also apply to the aryl moiety in aryloxy, arylthio (=arylsulfanyl), monoarylamino and diarylamino.

In the context of the present invention, the expression “heterocycloalkyl” comprises nonaromatic, unsaturated or fully saturated, cycloaliphatic groups having generally 5 to 8 ring atoms, preferably 5 or 6 ring atoms. In the heterocycloalkyl groups, compared to the corresponding cycloalkyl groups, 1, 2, 3, 4 or more than 4 of the ring carbon atoms are replaced by heteroatoms or heteroatom-containing groups. The heteroatoms or heteroatom-containing groups are preferably selected from —O—, —S—, —NR^{b}—, —C(═O)—, —S(═O)— and/or —S(═O)₂—. R^{b} is preferably hydrogen, alkyl, cycloalkyl, heterocycloalkyl, aryl or hetaryl. Examples of heterocycloalkyl groups are especially pyrrolidinyl, piperidinyl, 2,2,6,6-tetramethylpiperidinyl, imidazolidinyl, pyrazolidinyl, oxazolidinyl, morpholinyl, thiazolidinyl, isothiazolidinyl, isoxazolidinyl, piperazinyl, tetrahydrothiophenyl, dihydrothien-2-yl, tetrahydrofuranyl, dihydrofuran-2-yl, tetrahydropyranyl, 2-oxazolinyl, 3-oxazolinyl, 4-oxazolinyl and dioxanyl.

Substituted heterocycloalkyl groups may, depending on the ring size, have one or more (e.g. 1, 2, 3, 4, 5 or more than 5) substituents. These are preferably each independently selected from alkyl, alkoxy, alkylthio, cycloalkyl, heterocycloalkyl, aryl, hetaryl, fluorine, chlorine, bromine, hydroxyl, mercapto, cyano, nitro, nitroso, formyl, acyl, COOH, carboxylate, alkylcarbonyloxy, carbamoyl, SO₃H, sulfonate, sulfamino, sulfamide, amidino, NE¹¹E¹² where E¹¹ and E¹² are each independently hydrogen, alkyl, cycloalkyl, heterocycloalkyl, aryl or hetaryl. In the case of substitution, the heterocycloalkyl groups preferably bear one or more, for example one, two, three, four or five, C₁-C₆-alkyl groups.

The above remarks regarding heterocycloalkyl also apply to the heterocycloalkyl moiety in heterocycloalkoxy, heterocycloalkylthio (=heterocycloalkylsulfanyl), monoheterocycloalkylamino and diheterocycloalkylamino.

In the context of the present invention, the expression “hetaryl” (heteroaryl) comprises heteroaromatic, mono- or polycyclic groups. In addition to the ring carbon atoms, these have 1, 2, 3, 4 or more than 4 heteroatoms as ring members. The heteroatoms are preferably selected from oxygen, nitrogen, selenium and sulfur. The hetaryl groups have preferably 5 to 18, e.g. 5, 6, 8, 9, 10, 11, 12, 13 or 14, ring atoms.

Monocyclic hetaryl groups are preferably 5- or 6-membered hetaryl groups, such as 2-furyl (furan-2-yl), 3-furyl (furan-3-yl), 2-thienyl (thiophen-2-yl), 3-thienyl (thiophen-3-yl), selenophen-2-yl, selenophen-3-yl, 1H-pyrrol-2-yl, 1H-pyrrol-3-yl, pyrrol-1-yl, imidazol-2-yl, imidazol-1-yl, imidazol-4-yl, pyrazol-1-yl, pyrazol-3-yl, pyrazol-4-yl, pyrazol-5-yl, 3-isoxazolyl, 4-isoxazolyl, 5-isoxazolyl, 3-isothiazolyl, 4-isothiazolyl, 5-isothiazolyl, 2-oxazolyl, 4-oxazolyl, 5-oxazolyl, 2-thiazolyl, 4-thiazolyl, 5-thiazolyl, 1,2,4-oxadiazol-3-yl, 1,2,4-oxadiazol-5-yl, 1,3,4-oxadiazol-2-yl, 1,2,4-thiadiazol-3-yl, 1,2,4-thiadiazol-5-yl, 1,3,4-thiadiazol-2-yl, 4H-[1,2,4]-triazol-3-yl, 1,3,4-triazol-2-yl, 1,2,3-triazol-1-yl, 1,2,4-triazol-1-yl, pyridin-2-yl, pyridin-3-yl, pyridin-4-yl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl.

Polycyclic hetaryl groups have 2, 3, 4 or more than 4 fused rings. The fused-on rings may be aromatic, saturated or partly unsaturated. Examples of polycyclic hetaryl groups are quinolinyl, isoquinolinyl, indolyl, isoindolyl, indolizinyl, benzofuranyl, isobenzofuranyl, benzothiophenyl, benzoxazolyl, benzisoxazolyl, benzthiazolyl, benzoxadiazolyl, benzothiadiazolyl, benzoxazinyl, benzopyrazolyl, benzimidazolyl, benzotriazolyl, benzotriazinyl, benzoselenophenyl, thienothiophenyl, thienopyrimidyl, thiazolothiazolyl, dibenzopyrrolyl (carbazolyl), dibenzofuranyl, dibenzothiophenyl, naphtho[2,3-b]thiophenyl, naphtha[2,3-b]furyl, dihydroindolyl, dihydroindolizinyl, dihydroisoindolyl, dihydroquinolinyl and dihydroisoquinolinyl.

Substituted hetaryl groups may, depending on the number and size of their ring systems, have one or more (e.g. 1, 2, 3, 4, 5 or more than 5) substituents. These are preferably each independently selected from alkyl, alkoxy, alkylthio, cycloalkyl, heterocycloalkyl, aryl, hetaryl, fluorine, chlorine, bromine, hydroxyl, mercapto, cyano, nitro, nitroso, formyl, acyl, COOH, carboxylate, alkylcarbonyloxy, carbamoyl, SO₃H, sulfonate, sulfamino, sulfamide, amidino, NE¹³E¹⁴ where E¹³ and E¹⁴ are each independently hydrogen, alkyl, cycloalkyl, heterocycloalkyl, aryl or hetaryl. Halogen substituents are preferably fluorine, chlorine or bromine. The substituents are preferably selected from C₁-C₆-alkyl, C₁-C₆-alkoxy, hydroxyl, carboxyl, halogen and cyano.

The above remarks regarding hetaryl also apply to the hetaryl moiety in hetaryloxy, hetarylthio, monohetarylamino and dihetarylamino.

For the purposes of the present invention, the expression “acyl” refers to alkanoyl or aroyl groups which generally have from 2 to 11, preferably from 2 to 8, carbon atoms, for example the acetyl, propanoyl, butanoyl, pentanoyl, hexanoyl, heptanoyl-, 2-ethyl-hexanoyl, 2-propylheptanoyl, pivaloyl, benzoyl or naphthoyl group.

The groups NE¹E², NE³E⁴, NE⁵E⁶, NE⁷E⁸, NE⁹E¹⁰, NE¹¹E¹² and NE¹³E¹⁴ are preferably N,N-dimethylamino, N,N-diethylamino, N,N-dipropylamino, N,N-diisopropylamino, N,N-di-n-butylamino, N,N-di-t-butylamino, N,N-dicyclohexylamino or N,N-diphenylamino.

Fused ring systems can comprise alicyclic, aliphatic heterocyclic, aromatic and heteroaromatic rings and combinations thereof, hydroaromatic joined by fusion. Fused ring systems comprise two, three or more (e.g. 4, 5, 6, 7 or 8) rings. Depending on the way in which the rings in fused ring systems are joined, a distinction is made between ortho-fusion, i.e. each ring shares at least one edge or two atoms with each adjacent ring, and peri-fusion in which a carbon atom belongs to more than two rings. Preferred fused ring systems are ortho-fused ring systems.

In a special embodiment, the compounds of the invention are compounds of the formula (I), wherein
X¹ and X² are independently of one another selected from O and C(CN)₂;
R¹, R^{2a}, R^{2b}, R^{3a} and R^{3b} are independently of one another selected from hydrogen, F, Cl, Br, I, CN, hydroxy, mercapto, NE¹E², where E¹ and E² are each independently selected from hydrogen, alkyl, cycloalkyl and aryl,
   in each case unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, aryl, aryloxy, arylthio, (monoaryl)amino and (diaryl)amino;
A is selected from the groups of the general formulae (II.1), (II.2), (II.3), (II.4), (II.5) and (II.6)

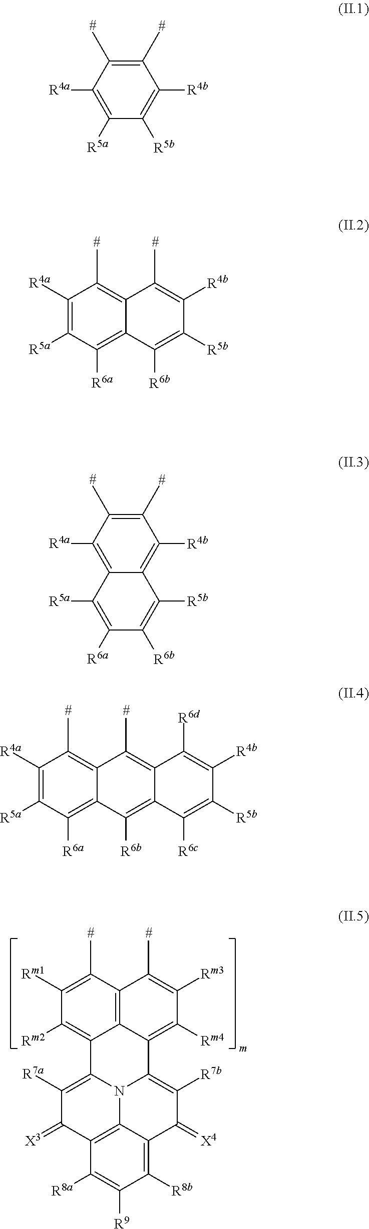

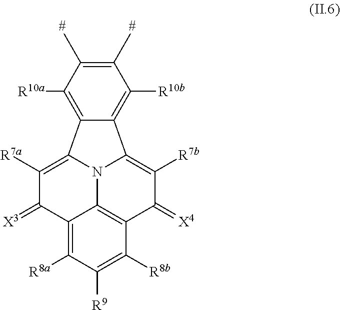

in which
# in each case represents the bonding site to the cyclazine skeleton;
in the formulae (II.1), (II.2), (II.3), (II.4)
R^{4a}, R^{4b}, R^{5a}, R^{5b}, and, if present, R^{6a}, R^{6b}, R^{6c} and R^{6d} are independently of one another selected from hydrogen, F, Cl, Br, I, CN, hydroxy, mercapto, formyl, acyl, carboxy, carboxylate, alkylcarbonyloxy, sulfo, sulfonate, sulfoamino, alkylsulfonyl, arylsulfonyl, NE¹E², where E¹ and E² are each independently selected from hydrogen, alkyl, cycloalkyl and aryl,
   in each case unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, aryl, aryloxy, arylthio, (monoaryl)amino and (diaryl)amino;
m in the formula (II.5) is 1, 2, 3 or 4;
in the formulae (II.5) and (II.6)
X³ and X⁴ are independently of one another selected from O and C(CN)₂;
R^{7a}, R^{7b}, R^{8a}, R^{8b}, R⁹ and, if present, each R^{m1}, R^{m2}, R^{m3} and R^{m4} are independently of one another selected from hydrogen, F, Cl, Br, I, CN, hydroxy, mercapto, NE¹E², where E¹ and E² are each independently selected from hydrogen, alkyl, cycloalkyl and aryl,
   in each case unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, aryl, aryloxy, arylthio, (monoaryl)amino and (diaryl)amino;
R^{10a}, R^{10b}, if present, are independently of one another selected from hydrogen, F, Cl, Br, I, CN, hydroxy, mercapto, formyl, acyl, carboxy, carboxylate, alkylcarbonyloxy, sulfo, sulfonate, sulfoamino, alkylsulfonyl, arylsulfonyl, NE¹E², where E¹ and E² are each independently selected from hydrogen, alkyl, cycloalkyl and aryl,
   in each case unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, aryl, aryloxy, arylthio, (monoaryl)amino and (diaryl)amino.

### Group of Embodiments 1

Preferably, group A is selected from groups of the general formulae (II.1), (II.2), (II.3) and (II.4)

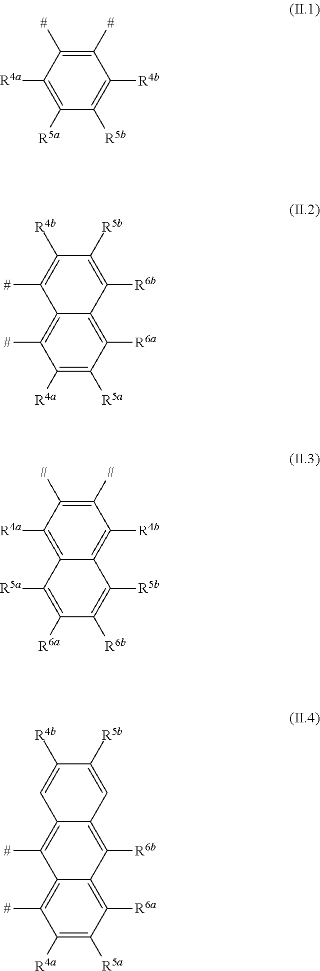

in which
# in each case represents the bonding site to the cyclazine skeleton,
R^{4a}, R^{4b}, R^{5a}, R^{5b}, and, if present, R^{6a}, R^{6b}, R^{6c} and R^{6d} are independently of one another selected from hydrogen, F, Cl, Br, I, CN, hydroxy, mercapto, nitro, cyanato, thiocyanato, formyl, acyl, carboxy, carboxylate, alkylcarbonyloxy, carbamoyl, alkylaminocarbonyl, dialkylaminocarbonyl, sulfo, sulfonate, sulfoamino, sulfamoyl, alkylsulfonyl, arylsulfonyl, amidino, NE¹E², where E¹ and E² are each independently selected from hydrogen, alkyl, cycloalkyl, heterocycloalkyl, aryl or hetaryl,
   in each case unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, heterocycloalkyl, heterocycloalkoxy, heterocycloalkylthio, (monoheterocycloalkyl)amino, (diheterocycloalkyl)amino, aryl, aryloxy, arylthio, (monoaryl)amino, (diaryl)amino, hetaryl, hetaryloxy, hetarylthio, (monohetaryl)amino and (dihetaryl)amino.

More preferably, group A is selected from groups of the general formulae (II.1) and (II.2). The compound of the general formula (I) is then especially selected from compounds of the formulae (I.A), i.e. compounds of formula (I), wherein A is a group of the formula (II.1) and (I.B), i.e. compounds of formula (I), wherein A is a group of formula (II.2).

The compounds of the general formula (I), wherein group A is selected from groups of the general formulae (II.1), (II.2), (II.3) and (II.4), the compounds of the formula (I.A) and the compounds of the formula (I.B) are denoted in the following also as “group of embodiments 1”. All definitions of substituents and variables regarding the group of embodiments 1, where applicable, refer likewise to the compounds of the general formula (I), wherein group A is selected from groups of the general formulae (II.1), (II.2), (II.3) and (II.4), the compounds of the formula (I.A) and the compounds of the formula (I.B).

In the compounds of the formula (I) according to the group of embodiments 1, the R^{2a} and R^{2b} radicals may have identical or different definitions. Preferably, the R^{2a} and R^{2b} radicals have identical definitions. In the compounds of the formula (I) according to the group of embodiments 1, the R^{3a} and R^{3b} radicals may have identical or different definitions. Preferably, the R^{3a} and R^{3b} radicals have identical definitions. In the compounds of the formula (I) according to the group of embodiments 1, the R^{4a} and R^{4b} radicals may have identical or different definitions. Preferably, the R^{4a} and R^{4b} radicals have identical definitions. In the compounds of the formula (I) according to the group of embodiments 1, the R^{5a} and R^{5b} radicals may have identical or different definitions. Preferably, the R^{5a} and R^{5b} radicals have identical definitions. In the compounds of the formula (I) according to the group of embodiments 1, the R^{6a} and R^{6b} radicals may have identical or different definitions. Preferably, the R^{6a} and R^{6b} radicals have identical definitions.

In a special embodiment, group A is a group of the general formula (II.1), wherein R^{4a}, R^{4b}, R^{5a} and R^{5b} are all hydrogen.

In a further special embodiment, group A is a group of the general formula (II.2), wherein R^{4a}, R^{4b}, R^{5a}, R^{5b}, R^{6a} and R^{6b} are all hydrogen.

Preferably, in the group of embodiments 1 X¹ and X² have the same meaning.

Preferably, the radicals R¹, R^{2a}, R^{2b}, R^{3a}, R^{3b}, R^{4a}, R^{4b}, R^{5a}, R^{5b}, and, if present, R^{6a}, R^{6b}, R^{6c} and R^{6d} are independently of one another selected from hydrogen, linear C₁-C₃₀-alkyl, branched C₃-C₃₀-alkyl, perfluoro-C₁-C₃₀-alkyl, 1H,1H-perfluoro-C₂-C₃₀-alkyl, 1H,1H,2H,2H-perfluoro-C₃-C₃₀-alkyl, a radical of the formula G.1, a radical of the formula G.2 and a radical of the formula G.3

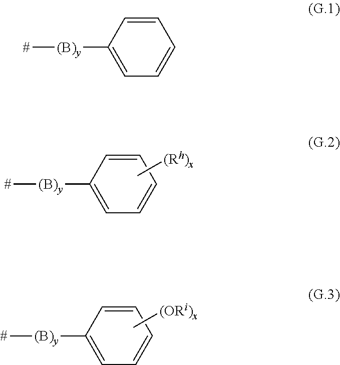

where
# represents the bonding side to the remainder of the molecule,
B where present, is selected from O, S and a C₁-C₁₀-alkylene group which may be interrupted by one or more nonadjacent groups which are selected from —O— and —S—,
y is 0 or 1,
R^{h} is independently of one another selected from C₁-C₃₀-alkyl, C₁-C₃₀-fluoroalkyl, fluorine, chlorine, bromine, NE³E⁴, nitro and cyano, where E³ and E⁴, independently of one another, are hydrogen, alkyl, cycloalkyl, heterocycloalkyl, aryl or hetaryl,
Rⁱ is independently of one another selected from C₁-C₃₀-alkyl,
x in formulae G.2 and G.3 is 1, 2, 3, 4 or 5.

If B is present, i.e. if y is 1, the variable B is preferably O or a C₁-C₁₀-alkylene group.

Irrespectively of its occurrence, R^{h} is preferably selected from C₁-C₃₀-alkyl.

Irrespectively of its occurrence, Rⁱ is preferably selected from C₁-C₃₀-alkyl.

In a preferred embodiment, at least one of the radicals R¹, R^{2a}, R^{2b}, R^{3a}, R^{3b}, R^{4a}, R^{4b}, R^{5a}, R^{5b}, and, if present, R^{6a}, R^{6b}, R^{6c} and R^{6d} is a linear C₁-C₃₀-alkyl radical. Preferred linear alkyl groups are methyl, ethyl, n-propyl, n-butyl, n-pentyl, n-hexyl, n-heptyl, n-octyl, n-nonyl, n-decyl, n-undecyl, n-dodecyl, n-tridecyl, n-tetradecyl, n-hexadecyl, n-octadecyl and n-eicosyl.

In a special embodiment, R¹ is a linear C₁-C₃₀-alkyl radical. According to this special embodiment, R¹ is preferably a linear C₁-C₁₀-alkyl. According to this embodiment, preferably R^{2a}, R^{2b}, R^{3a}, R^{3b}, R^{4a}, R^{4b}, R^{5a}, R^{5b}, and, if present, R^{6a}, R^{6b}, R^{6c} and R^{6d} are all hydrogen. According to this embodiment, likewise preferably R^{2a}, R^{2b}, R^{3a} and R^{3b} are all hydrogen and at least one of the radicals R^{4a}, R^{4b}, R^{5a}, R^{5b}, and, if present, R^{6a}, R^{6b}, R^{6c} and R^{6d} is selected from fluorine, chlorine, cyano, C₁-C₁₀-alkyl and aryl, which is unsubstituted or carries one, two or three C₁-C₁₀-alkyl radicals and the other radicals R^{4a}, R^{4b}, R^{5a}, R^{5b}, and, if present, R^{6a}, R^{6b}, R^{6c} and R^{6d} are all hydrogen.

In a preferred embodiment, at least one of the radicals R¹, R^{2a}, R^{2b}, R^{3a}, R^{3b}, R^{4a}, R^{4b}, R^{5a}, R^{5b}, and, if present, R^{6a}, R^{6b}, R^{6c} and R^{6d} is a branched C₃-C₃₀-alkyl radical.

In a special embodiment, R¹ is a branched C₃-C₃₀-alkyl radical. According to this embodiment, preferably R^{2a}, R^{2b}, R^{3a}, R^{3b}, R^{4a}, R^{4b}, R^{5a}, R^{5b}, and, if present, R^{6a}, R^{6b}, R^{6c} and R^{6d} are all hydrogen. According to this embodiment, likewise preferably R^{2a}, R^{2b}, R^{3a}, R^{3b} are all hydrogen and at least one of the radicals R^{4a}, R^{4b}, R^{5a}, R^{5b}, and, if present, R^{6a}, R^{6b}, R^{6c} and R^{6d} is selected from fluorine, chlorine, cyano, C₁-C₁₀-alkyl and aryl, which is unsubstituted or carries one, two or three C₁-C₁₀-alkyl radicals and the other radicals R^{4a}, R^{4b}, R^{5a}, R^{5b}, and, if present, R^{6a}, R^{6b}, R^{6c} and R^{6d} are all hydrogen.

Preferably at least one of the radicals R¹, R^{2a}, R^{2b}, R^{3a}, R^{3b}, R^{4a}, R^{4b}, R^{5a}, R^{5b}, and, if present, R^{6a}, R^{6b}, R^{6c} and R^{6d} is selected from radicals of the general formulae (III.1) and (III.2)

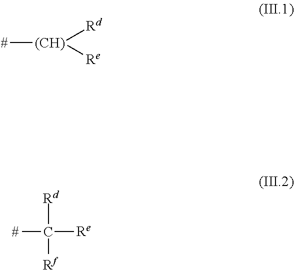

in which
# is a bonding site, and
   in the formula (III.1) R^{d} and R^{e} are independently selected from C₁- to C₂₈-alkyl, where the sum of the carbon atoms of the R^{d} and R^{e} radicals is an integer from 2 to 29,
   in the formula (III.2) R^{d}, R^{e} and R^{f} are independently selected from C₁- to C₂₇-alkyl, where the sum of the carbon atoms of the R^{d}, R^{e} and R^{f} radicals is an integer from 3 to 29.

In a preferred embodiment of the compounds (I), the radical R¹ is a radical of the formula (III.1) or (III.2). According to this embodiment, preferably R^{2a}, R^{2b}, R^{3a}, R^{3b}, R^{4a}, R^{4b}, R^{5a}, R^{5b}, and, if present, R^{6a}, R^{6b}, R^{6c} and R^{6d} are all hydrogen.

Preferably, in the formulae (III.1) and (III.2), the R^{d}, R^{e} and R^{f} radicals are independently selected from C₁- to C₁₂-alkyl, especially C₁- to C₈-alkyl.

Preferred radicals of the formula (III.1) are:

1-ethylpropyl, 1-methylpropyl, 1-propylbutyl, 1-ethylbutyl, 1-methylbutyl, 1-butylpentyl, 1-propylpentyl, 1-ethylpentyl, 1-methylpentyl, 1-pentylhexyl, 1-butylhexyl, 1-propylhexyl, 1-ethylhexyl, 1-methylhexyl, 1-hexylheptyl, 1-pentylheptyl, 1-butylheptyl, 1-propylheptyl, 1-ethylheptyl, 1-methylheptyl, 1-heptyloctyl, 1-hexyloctyl, 1-pentyloctyl, 1-butyloctyl, 1-propyloctyl, 1-ethyloctyl, 1-methyloctyl, 1-octylnonyl, 1-heptylnonyl, 1-hexylnonyl, 1-pentylnonyl, 1-butylnonyl, 1-propylnonyl, 1-ethylnonyl, 1-methylnonyl, 1-nonyldecyl, 1-octyldecyl, 1-heptyldecyl, 1-hexyldecyl, 1-pentyldecyl, 1-butyldecyl, 1-propyldecyl, 1-ethyldecyl, 1-methyldecyl, 1-decylundecyl, 1-nonylundecyl, 1-octylundecyl, 1-heptylundecyl, 1-hexylundecyl, 1-pentylundecyl, 1-butylundecyl, 1-propylundecyl, 1-ethylundecyl, 1-methylundecyl, 1-undecyldodecyl, 1-decyldodecyl, 1-nonyldodecyl, 1-octyldodecyl, 1-heptyldodecyl, 1-hexyldodecyl, 1-pentyldodecyl, 1-butyldodecyl, 1-propyldodecyl, 1-ethyldodecyl, 1-methyldodecyl, 1-dodecyltridecyl, 1-undecyltridecyl, 1-decyltridecyl, 1-nonyltridecyl, 1-octyltridecyl, 1-heptyltridecyl, 1-hexyltridecyl, 1-pentyltridecyl, 1-butyltridecyl, 1-propyltridecyl, 1-ethyltridecyl, 1-methyltridecyl, 1-tridecyltetradecyl, 1-undecyltetradecyl, 1-decyltetradecyl, 1-nonyltetradecyl, 1-octyltetradecyl, 1-heptyltetradecyl, 1-hexyltetradecyl, 1-pentyltetradecyl, 1-butyltetradecyl, 1-propyltetradecyl, 1-ethyltetradecyl, 1-methyltetradecyl, 1-pentadecylhexadecyl, 1-tetradecylhexadecyl, 1-tridecylhexadecyl, 1-dodecylhexadecyl, 1-undecylhexadecyl, 1-decylhexadecyl, 1-nonylhexadecyl, 1-octylhexadecyl, 1-heptylhexadecyl, 1-hexylhexadecyl, 1-pentylhexadecyl, 1-butylhexadecyl, 1-propylhexadecyl, 1-ethylhexadecyl, 1-methylhexadecyl, 1-hexadecyloctadecyl, 1-pentadecyloctadecyl, 1-tetradecyloctadecyl, 1-tridecyloctadecyl, 1-dodecyloctadecyl, 1-undecyloctadecyl, 1-decyloctadecyl, 1-nonyloctadecyl, 1-octyloctadecyl, 1-heptyloctadecyl, 1-hexyloctadecyl, 1-pentyloctadecyl, 1-butyloctadecyl, 1-propyloctadecyl, 1-ethyloctadecyl, 1-methyloctadecyl, 1-nonadecyleicosanyl, 1-octadecyleicosanyl, 1-heptadecyleicosanyl, 1-hexadecyleicosanyl, 1-pentadecyleicosanyl, 1-tetradecyleicosanyl, 1-tridecyleicosanyl, 1-dodecyleicosanyl, 1-undecyleicosanyl, 1-decyleicosanyl, 1-nonyleicosanyl, 1-octyleicosanyl, 1-heptyleicosanyl, 1-hexyleicosanyl, 1-pentyleicosanyl, 1-butyleicosanyl, 1-propyleicosanyl, 1-ethyleicosanyl, 1-methyleicosanyl, 1-eicosanyldocosanyl, 1-nonadecyldocosanyl, 1-octadecyldocosanyl, 1-heptadecyldocosanyl, 1-hexadecyldocosanyl, 1-pentadecyldocosanyl, 1-tetradecyldocosanyl, 1-tridecyldocosanyl, 1-undecyldocosanyl, 1-decyldocosanyl, 1-nonyldocosanyl, 1-octyldocosanyl, 1-heptyldocosanyl, 1-hexyldocosanyl, 1-pentyldocosanyl, 1-butyldocosanyl, 1-propyldocosanyl, 1-ethyldocosanyl, 1-methyldocosanyl, 1-tricosanyltetracosanyl, 1-docosanyltetracosanyl, 1-nonadecyltetracosanyl, 1-octadecyltetracosanyl, 1-heptadecyltetracosanyl, 1-hexadecyltetracosanyl, 1-pentadecyltetracosanyl, 1-pentadecyltetracosanyl, 1-tetradecyltetracosanyl, 1-tridecyltetracosanyl, 1-dodecyltetracosanyl, 1-undecyltetracosanyl, 1-decyltetracosanyl, 1-nonyltetracosanyl, 1-octyltetracosanyl, 1-heptyltetracosanyl, 1-hexyltetracosanyl, 1-pentyltetracosanyl, 1-butyltetracosanyl, 1-propyltetracosanyl, 1-ethyltetracosanyl, 1-methyltetracosanyl, 1-heptacosanyloctacosanyl, 1-hexacosanyloctacosanyl, 1-pentacosanyloctacosanyl, 1-tetracosanyloctacosanyl, 1-tricosanyloctacosanyl, 1-docosanyloctacosanyl, 1-nonadecyloctacosanyl, 1-octadecyloctacosanyl, 1-heptadecyloctacosanyl, 1-hexadecyloctacosanyl, 1-hexadecyloctacosanyl, 1-pentadecyloctacosanyl, 1-tetradecyloctacosanyl, 1-tridecyloctacosanyl, 1-dodecyloctacosanyl, 1-undecyloctacosanyl, 1-decyloctacosanyl, 1-nonyloctacosanyl, 1-octyloctacosanyl, 1-heptyloctacosanyl, 1-hexyloctacosanyl, 1-pentyloctacosanyl, 1-butyloctacosanyl, 1-propyloctacosanyl, 1-ethyloctacosanyl, 1-methyloctacosanyl.

Particularly preferred radicals of the formula (III.1) are:

1-methylethyl, 1-methylpropyl, 1-methylbutyl, 1-methylpentyl, 1-methylhexyl, 1-methylheptyl, 1-methyloctyl, 1-ethylpropyl, 1-ethylbutyl, 1-ethylpentyl, 1-ethylhexyl, 1-ethylheptyl, 1-ethyloctyl, 1-propylbutyl, 1-propylpentyl, 1-propylhexyl, 1-propylheptyl, 1-propyloctyl, 1-butylpentyl, 1-butylhexyl, 1-butylheptyl, 1-butyloctyl, 1-pentylhexyl, 1-pentylheptyl, 1-pentyloctyl, 1-hexylheptyl, 1-hexyloctyl, 1-heptyloctyl.

A particularly preferred radical of the formula (III.2) is tert.-butyl.

Preferably at least one of the radicals R¹, R^{2a}, R^{2b}, R^{3a}, R^{3b}, R^{4a}, R^{4b}, R^{5a}, R^{5b}, and, if present, R^{6a}, R^{6b}, R^{6c} and R^{6d} is selected from perfluoro-C₁-C₃₀-alkyl, 1H,1H-perfluoro-C₂-C₃₀-alkyl or 1H,1H,2H,2H-perfluoro-C₃-C₃₀-alkyl.

In a preferred embodiment of the compounds (I), the radical R¹ is selected from perfluoro-C₁-C₃₀-alkyl, 1H,1H-perfluoro-C₂-C₃₀-alkyl or 1H,1H,2H,2H-perfluoro-C₃-C₃₀-alkyl. According to this embodiment, preferably R^{2a}, R^{2b}, R^{3a}, R^{3b}, R^{4a}, R^{4b}, R^{5a}, R^{5b}, and, if present, R^{6a}, R^{6b}, R^{6c} and R^{6d} are all hydrogen.

In another preferred embodiment, at least one of the radicals R^{4a}, R^{4b}, R^{5a}, R^{5b}, and, if present, R^{6a}, R^{6b}, R^{6c} and R^{6d} is selected from fluorine, chlorine, cyano, C₁-C₁₀-alkyl or aryl, which is unsubstituted or carries one, two or three C₁-C₁₀-alkyl radicals.

In a preferred embodiment, at least one of the radicals R¹, R^{2a}, R^{2b}, R^{3a}, R^{3b}, R^{4a}, R^{4b}, R^{5a}, R^{5b}, and, if present, R^{6a}, R^{6b}, R^{6c} and R^{6d} is selected from CF₃, C₂F₅, n-C₃F₇, n-C₄F₉, n-C₅F₁₁,n-C₆F₁₃, CF(CF₃)₂, C(CF₃)₃, CF₂CF(CF₃)₂, CF(CF₃)(C₂F₅), CH₂—CF₃, CH₂—C₂F₅, CH₂-(n-C₃F₇), CH₂-(n-C₄F₉), CH₂-(n-C₅F₁₁), CH₂-(n-C₆F₁₃), CH₂—CF(CF₃)₂, CH₂—C(CF₃)₃, CH₂—CF₂CF(CF₃)₂, CH₂—CF(CF₃)(C₂F₅), CH₂—CH₂—CF₃, CH₂—CH₂—C₂F₅, CH₂—CH₂-(n-C₃F₇), CH₂—CH₂-(n-C₄F₉), CH₂—CH₂-(n-C₅F₁₁), CH₂—CH₂-(n-C₆F₁₃), CH₂—CH₂—CF(CF₃)₂, CH₂—CH₂—C(CF₃)₃, CH₂—CH₂—CF₂CF(CF₃)₂ and CH₂—CH₂—CF(CF₃)(C₂F₅). In an especially preferred embodiment of the compounds (I), the radical R¹ is selected from the aforementioned radicals. In an especially more preferred embodiment, R¹ is hydrogen, linear C₁-C₁₀-alkyl or branched C₃-C₂₀-alkyl. According to this embodiment, preferably R^{2a}, R^{2b}, R^{3a}, R^{3b}, R^{4a}, R^{4b}, R^{5a}, R^{5b}, and, if present, R^{6a}, R^{6b}, R^{6c} and R^{6d} are all hydrogen.

Some particularly preferred compounds (I) are specified below:

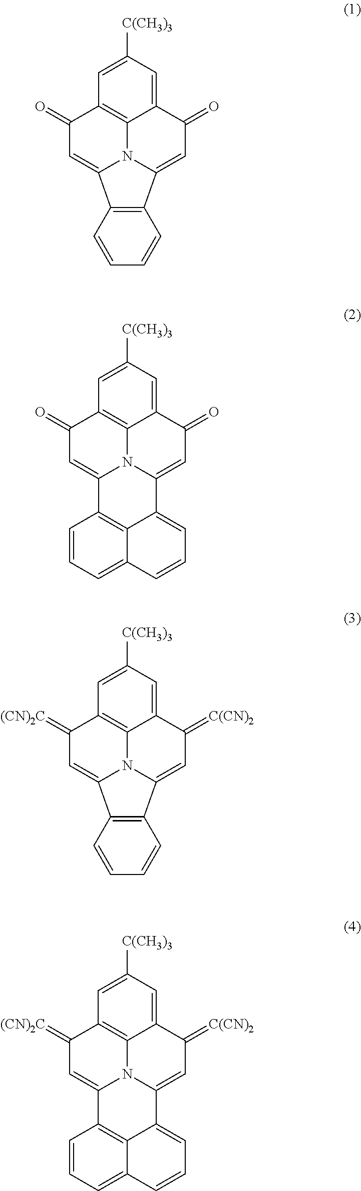

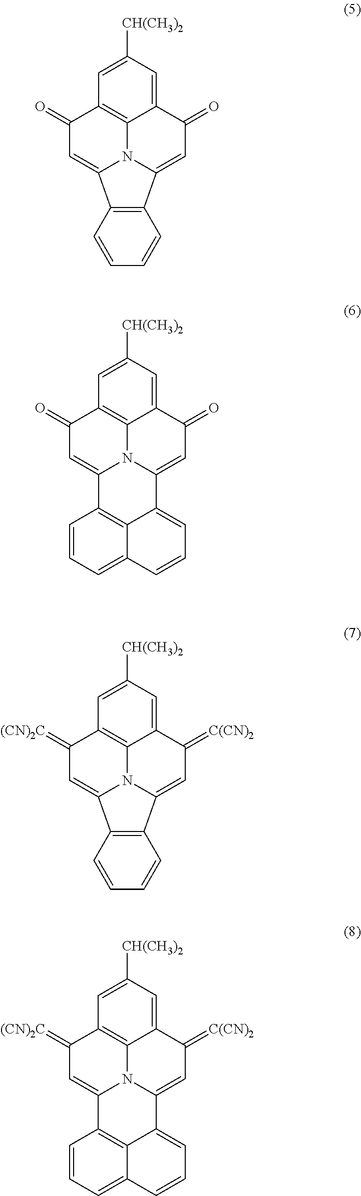

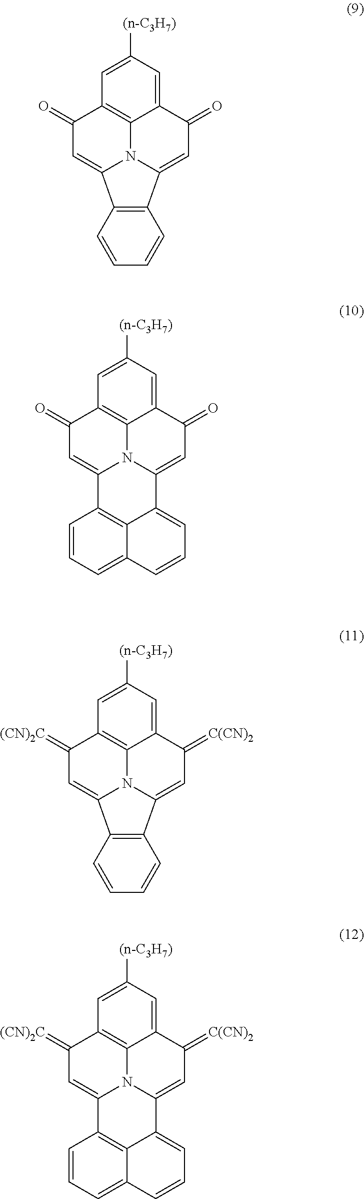

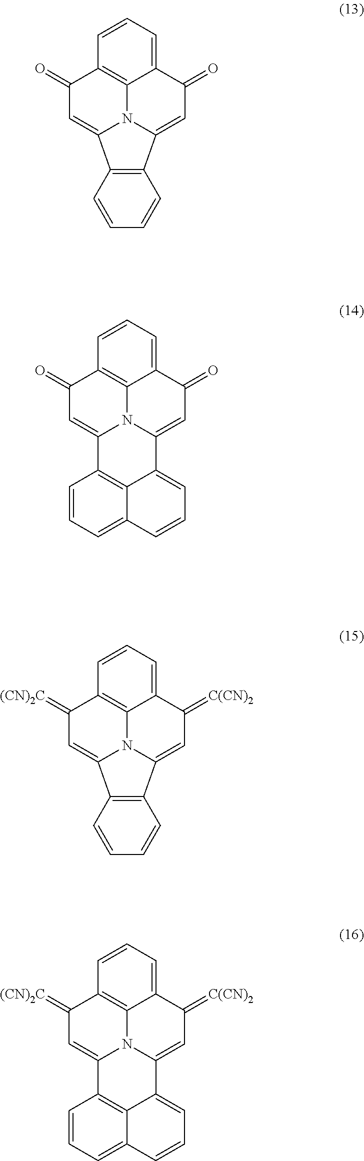

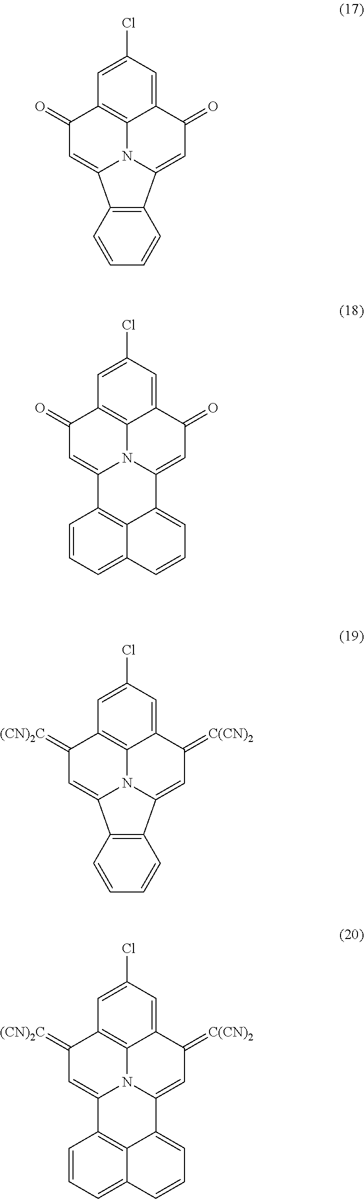

| 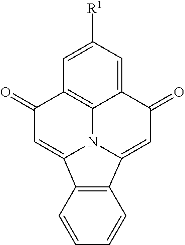 | 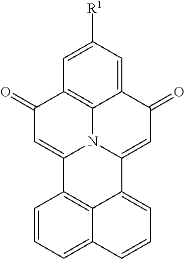 |
|---|---|
| (21a) R¹ = CH₃ | (22a) R¹ = CH₃ |
| (21b) R¹ = C₂H₅ | (22b) R¹ = C₂H₅ |
| (21c) R¹ = CH₂CH₂CH₂CH₃ | (22c) R¹ = CH₂CH₂CH₂CH₃ |
| (21d) R¹ = CH(C₂H₅)CH₃ | (22d) R¹ = CH(C₂H₅)CH₃ |
| (21e) R¹ = CH₂CH(CH₃)₂ | (22e) R¹ = CH₂CH(CH₃)₂ |
| (21f) R¹ = CF₃ | (22f) R¹ = CF₃ |
| (21g) R¹ = C₂F₅ | (22g) R¹ = C₂F₅ |
| (21h) R¹ = CH₂CF₃ | (22h) R¹ = CH₂CF₃ |
| (21i) R¹ = CH₂CH₂CF₃ | (22i) R¹ = CH₂CH₂CF₃ |
| (21k) R¹ = CH₂C₂F₅ | (22k) R¹ = CH₂C₂F₅ |
| (21l) R¹ = n-C₁₀H₂₁ | (22l) R¹ = n-C₁₀H₂₁ |
| 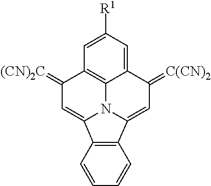 | 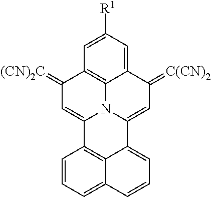 |
| (23a) R¹ = CH₃ | (24a) R¹ = CH₃ |
| (23b) R¹ = C₂H₅ | (24b) R¹ = C₂H₅ |
| (23c) R¹ = CH₂CH₂CH₂CH₃ | (24c) R¹ = CH₂CH₂CH₂CH₃ |
| (23d) R¹ = CH(C₂H₅)CH₃ | (24d) R¹ = CH(C₂H₅)CH₃ |
| (23e) R¹ = CH₂CH(CH₃)₂ | (24e) R¹ = CH₂CH(CH₃)₂ |
| (23f) R¹ = CF₃ | (24f) R¹ = CF₃ |
| (23g) R¹ = C₂F₅ | (24g) R¹ = C₂F₅ |
| (23h) R¹ = CH₂CF₃ | (24h) R¹ = CH₂CF₃ |
| (23i) R¹ = CH₂CH₂CF₃ | (24i) R¹ = CH₂CH₂CF₃ |
| (23k) R¹ = CH₂C₂F₅ | (24k) R¹ = CH₂C₂F₅ |
| (23l) R¹ = n-C₁₀H₂₁ | (24l) R¹ = n-C₁₀H₂₁ |
| 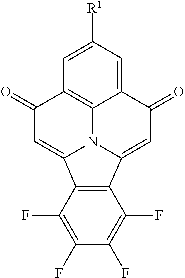 | 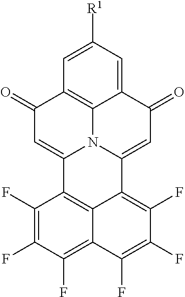 |
| (25a) R¹ = H | (26a) R¹ = H |
| (25b) R¹ = CH₃ | (26b) R¹ = CH₃ |
| (25c) R¹ = C₂H₅ | (26c) R¹ = C₂H₅ |
| (25d) R¹ = CH₂CH₂CH₃ | (26d) R¹ = CH₂CH₂CH₃ |
| (25e) R¹ = CH(CH₃)₂ | (26e) R¹ = CH(CH₃)₂ |
| (25f) R¹ = CH₂CH₂CH₂CH₃ | (26f) R¹ = CH₂CH₂CH₂CH₃ |
| (25g) R¹ = CH(C₂H₅)CH₃ | (26g) R¹ = CH(C₂H₅)CH₃ |
| (25h) R¹ = CH₂CH(CH₃)₂ | (26h) R¹ = CH₂CH(CH₃)₂ |
| (25i) R¹ = C(CH₃)₃ | (26i) R¹ = C(CH₃)₃ |
| (25k) R¹ = n-C₁₀H₂₁ | (26k) R¹ = n-C₁₀H₂₁ |
| 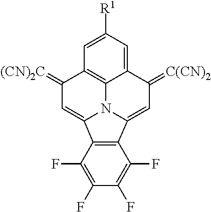 | 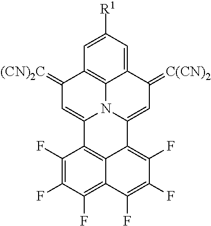 |
| (27a) R¹ = H | (28a) R¹ = H |
| (27b) R¹ = CH₃ | (28b) R¹ = CH₃ |
| (27c) R¹ = C₂H₅ | (28c) R¹ = C₂H₅ |
| (27d) R¹ = CH₂CH₂CH₃ | (28d) R¹ = CH₂CH₂CH₃ |
| (27e) R¹ = CH(CH₃)₂ | (28e) R¹ = CH(CH₃)₂ |
| (27f) R¹ = CH₂CH₂CH₂CH₃ | (28f) R¹ = CH₂CH₂CH₂CH₃ |
| (27g) R¹ = CH(C₂H₅)CH₃ | (28g) R¹ = CH(C₂H₅)CH₃ |
| (27h) R¹ = CH₂CH(CH₃)₂ | (28h) R¹ = CH₂CH(CH₃)₂ |
| (27i) R¹ = C(CH₃)₃ | (28i) R¹ = C(CH₃)₃ |
| (27k) R¹ = n-C₁₀H₂₁ | (27l) R¹ = n-C₁₀H₂₁ |

### Group of Embodiments 2

It is also preferred that in the compounds of the general formula (I) group A is selected from groups of the general formula (II.5),

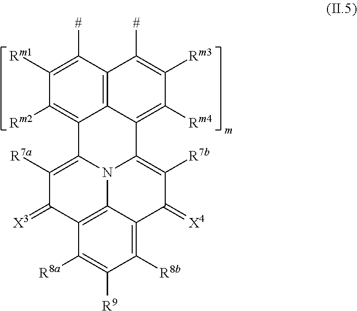

in which
# in each case represents the bonding site to the cyclazine skeleton,
m is 1, 2, 3 or 4,
X³ and X⁴ are independently of one another selected from O and C(CN)₂,
R^{7a}, R^{7b}, R^{8a}, R^{8b}, R⁹ and each R^{m1}, R^{m2}, R^{m3} and R^{m4} are independently of one another selected from hydrogen, F, Cl, Br, I, CN, hydroxy, mercapto, nitro, cyanato, thiocyanato, formyl, acyl, carboxy, carboxylate, alkylcarbonyloxy, carbamoyl, alkylaminocarbonyl, dialkylaminocarbonyl, sulfo, sulfonate, sulfoamino, sulfamoyl, alkylsulfonyl, arylsulfonyl, amidino, NE¹E², where E¹ and E² are each independently selected from hydrogen, alkyl, cycloalkyl, heterocycloalkyl, aryl or hetaryl,
   in each case unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, heterocycloalkyl, heterocycloalkoxy, heterocycloalkylthio, (monoheterocycloalkyl)amino, (diheterocycloalkyl)amino, aryl, aryloxy, arylthio, (monoaryl)amino, (diaryl)amino, hetaryl, hetaryloxy, hetarylthio, (monohetaryl)amino and (dihetaryl)amino.

The compounds of the general formula (I), wherein group A is selected from groups of the general formula (II.5), are also referred to as compounds of the formula (I.C) and are especially selected from compounds of the formula (I.Ca). The compounds of the formula (I.C) and the compounds of the formula (I.Ca) are denoted in the following also as “group of embodiments 2”.

In the compounds of the formula (I) according to the group of embodiments 2, the R¹ and R⁹ radicals may have identical or different definitions. In a preferred embodiment, the R¹ and R⁹ radicals have identical definitions. In the compounds of the formula (I) according to the group of embodiments 2, the R^{2a} and R^{2b} radicals may have identical or different definitions. In a preferred embodiment, the R^{2a} and R^{2b} radicals have identical definitions. In the compounds of the formula (I), the R^{3a} and R^{3b} radicals may have identical or different definitions. In a preferred embodiment, the R^{3a} and R^{3b} radicals have identical definitions. In the compounds of the formula (I), the R^{7a} and R^{7b} radicals may have identical or different definitions. In a preferred embodiment, the R^{7a} and R^{7b} radicals have identical definitions. In the compounds of the formula (I), the R^{8a} and R^{8b} radicals may have identical or different definitions. In a preferred embodiment, the R^{8a} and R^{8b} radicals have identical definitions.

Preferably, in the group of embodiments 2 X¹, X², X³ and X⁴ are all O or all C(CN)₂.

In the group of embodiments 2, preferably R¹ has one of the preferred meanings mentioned above for the group of embodiments 1.

In the group of embodiments 2, preferably R^{2a}, R^{2b}, R^{3a} and R^{3b} have one of the preferred meanings mentioned above for the group of embodiments 1. In particular R^{2a}, R^{2b}, R^{3a} and R^{3b} are each hydrogen.

In the group of embodiments 2, preferably the radicals R¹, R^{2a}, R^{2b}, R^{3a}, R^{3b}, R^{7a}, R^{7b}, R^{8a}, R^{8b}, R⁹ and each R^{m1}, R^{m2}, R^{m3} and R^{m4} are independently of one another selected from hydrogen, chlorine, bromine, linear C₁-C₃₀-alkyl, branched C₃-C₃₀-alkyl, perfluoro-C₁-C₃₀-alkyl, 1H,1H-perfluoro-C₂-C₃₀-alkyl, 1H,1H,2H,2H-perfluoro-C₃-C₃₀-alkyl, a radical of the formula G.1, a radical of the formula G.2 and a radical of the formula G.3

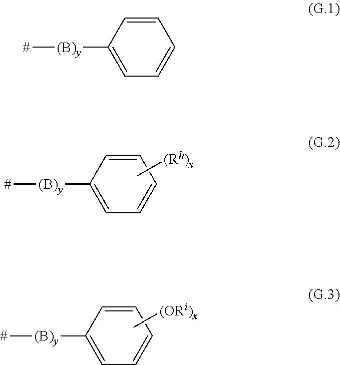

where
# represents the bonding side to the remainder of the molecule,
B where present, is selected from O, S and a C₁-C₁₀-alkylene group which may be interrupted by one or more nonadjacent groups which are selected from O and S,
y is 0 or 1,
R^{h} is independently of one another selected from C₁-C₃₀-alkyl, C₁-C₃₀-alkyl which carries a COOH group, C₁-C₃₀-fluoroalkyl, fluorine, chlorine, bromine, SO₃H, NE³E⁴, nitro and cyano, where E³ and E⁴, independently of one another, are hydrogen, alkyl, cycloalkyl, heterocycloalkyl, aryl or hetaryl,
Rⁱ is independently of one another selected from C₁-C₃₀-alkyl,
x in formulae G.2 and G.3 is 1, 2, 3, 4 or 5.

In the group of embodiments 2, the radicals R¹ and R⁹ are preferably independently of one another selected from hydrogen, chlorine, bromine, C₁-C₃₀-alkyl and C₁-C₃₀-haloalkyl.

In particular, R¹ and R⁹ are selected from
hydrogen,
chlorine, bromine,
linear C₁-C₃₀-alkyl,
branched C₃-C₃₀-alkyl,
perfluoro-C₁-C₃₀-alkyl,
1H,1H-perfluoro-C₂-C₃₀-alkyl, and
1H,1H,2H,2H-perfluoro-C₃-C₃₀-alkyl.

In particular, R¹ and R⁹ are selected from
hydrogen,
chlorine, bromine,
methyl, ethyl, n-propyl, n-butyl, n-pentyl, n-hexyl, n-heptyl, n-octyl, n-nonyl, n-decyl, n-undecyl, n-dodecyl, n-tridecyl, n-tetradecyl, n-hexadecyl, n-octadecyl, n-eicosyl,
branched C₃-C₃₀-alkyl, selected from radicals of the general formulae (III.1) and (III.2)

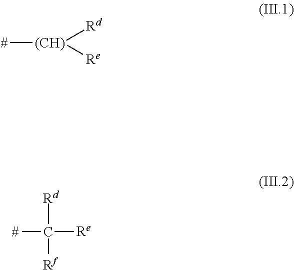

in which # is a bonding site to the remainder of the molecule, and in the formula (III.1) R^{d} and R^{e} are independently selected from C₁- to C₂₈-alkyl, where the sum of the carbon atoms of the R^{d} and R^{e} radicals is an integer from 2 to 29, in the formula (III.2) R^{d}, R^{e} and R^{f} are independently selected from C₁- to C₂₇-alkyl, where the sum of the carbon atoms of the R^{d}, R^{e} and R^{f} radicals is an integer from 3 to 29,
CF₃, C₂F₅, n-C₃F₇, n-C₄F₉, n-C₅F₁₁, n-C₆F₁₃, CF(CF₃)₂, C(CF₃)₃, CF₂CF(CF₃)₂, CF(CF₃)(C₂F₅),
CH₂—CF₃, CH₂—C₂F₅, CH₂-(n-C₃F₇), CH₂-(n-C₄F₉), CH₂-(n-C₅F₁₁), CH₂-(n-C₆F₁₃), CH₂—CF(CF₃)₂, CH₂—C(CF₃)₃, CH₂—CF₂CF(CF₃)₂, CH₂—CF(CF₃)(C₂F₅),
CH₂—CH₂—CF₃, CH₂—CH₂—C₂F₅, CH₂—CH₂-(n-C₃F₇), CH₂—CH₂-(n-C₄F₉), CH₂—CH₂-(n- C₅F₁₁), CH₂—CH₂-(n-C₆F₁₃), CH₂—CH₂—CF(CF₃)₂, CH₂—CH₂—C(CF₃)₃, CH₂—CH₂—CF₂CF(CF₃)₂ and CH₂—CH₂—CF(CF₃)(C₂F₅).

In a more preferred embodiment, the radicals R¹ and R⁹ are independently of one another selected from hydrogen, chlorine, bromine, a radical of the formula (III.1) or a radical of the formula (III.2). In the context of the formulae (III.1) and (III.2), the R^{d}, R^{e} and R^{f} radicals are independently selected from C₁- to C₁₂-alkyl, especially C₁- to C₈-alkyl.

Examples of preferred radicals of the formula (III.1) are:

1-methylethyl, 1-methylpropyl, 1-methylbutyl, 1-methylpentyl, 1-methylhexyl, 1-methylheptyl, 1-methyloctyl, 1-ethylpropyl, 1-ethylbutyl, 1-ethylpentyl, 1-ethylhexyl, 1-ethylheptyl, 1-ethyloctyl, 1-propylbutyl, 1-propylpentyl, 1-propylhexyl, 1-propylheptyl, 1-propyloctyl, 1-butylpentyl, 1-butylhexyl, 1-butylheptyl, 1-butyloctyl, 1-pentylhexyl, 1-pentylheptyl, 1-pentyloctyl, 1-hexylheptyl, 1-hexyloctyl, 1-heptyloctyl.

A particularly preferred radical of the formula (III.2) is tert.-butyl.

More preferably, the radicals R¹ and R⁹ are independently of one another selected from from hydrogen, chlorine, bromine, linear C₁-C₁₀-alkyl and branched C₃-C₁₀-alkyl. Especially, R¹ and R⁹ have the same meaning and are selected from hydrogen, chlorine, bromine, linear C₁-C₁₀-alkyl and branched C₃-C₁₀-alkyl. Particularly preferred branched C₃-C₁₀-alkyl radicals are isopropyl and tert.-butyl.

In particular in the group of embodiments, R¹ and R⁹ are independently of one another selected from hydrogen, chlorine or branched C₃-C₁₀-alkyl. Especially, R¹ and R⁹ have the same meaning and are selected from hydrogen, chlorine or branched C₃-C₁₀-alkyl. Particularly preferred branched C₃-C₁₀-alkyl radicals are isopropyl and tert.-butyl.

In the group of embodiments 2, the radicals R^{2a}, R^{2b}, R^{8a} and R^{8b} may have identical or different definitions. Preferably, the radicals R^{2a}, R^{2b}, R^{8a} and R^{8b} have identical definitions. In particular, R^{2a}, R^{2b}, R^{8a} and R^{8b} are each hydrogen.

In the group of embodiments 2, the radicals R^{3a}, R^{3b}, R^{7a} and R^{7b} may have identical or different definitions. Preferably, the radicals R^{3a}, R^{3b}, R^{7a} and R^{7b} have identical definitions. In particular, R^{3a}, R^{3b}, R^{7a} and R^{7b} are each hydrogen.

Preferably, R^{m1}, R^{m2}, R^{m3} and R^{m4} are, independently of one another, selected from hydrogen, C₁-C₃₀-alkoxy, C₁-C₃₀-alkylsulfanyl, aryloxy and arylthio where the two last mentioned radicals are unsubstituted or carry 1, 2 or 3 substituents selected from SO₃H, C₁-C₁₀-alkoxy and C₁-C₁₀-alkyl which is unsubstituted or substituted by COOH.

More preferably, R^{m1}, R^{m2}, R^{m3} and R^{m4} are, independently of one another, selected from hydrogen, C₁-C₃₀-alkoxy, C₁-C₃₀-alkylsulfanyl, phenyloxy and phenylthio, where the two last mentioned radicals are unsubstituted or carry 1, 2 or 3 substituents selected from SO₃H, C₁-C₁₀-alkoxy, C₁-C₁₀-alkyl and C₁-C₁₀-alkyl substituted by COOH.

Most preferably, R^{m1}, R^{m2}, R^{m3} and R^{m4} are independently of one another, selected from hydrogen, phenoxy, 2,6-diisopropylphenoxy, 2,4-di-tert-butylphenoxy, 4-tert-octylphenoxy, 4-sulfophenoxy and 4-(carboxymethyl)phenoxy.

Preferably, in the group of embodiments 2, the variable m is 1, 2 or 3, more preferably 1 or 2. Especially, m is 2.

According to a further particular embodiment, m is 1 and R¹¹, R¹², R¹³ and R¹⁴ are as defined above.

According to a special embodiment, m is 1 and R¹¹, R¹², R¹³ and R¹⁴ are selected from the group consisting of hydrogen, C₁-C₃₀-alkoxy, C₁-C₃₀-alkylsulfanyl, phenyloxy and phenylthio, where the two last mentioned radicals are unsubstituted or carry 1, 2 or 3 substituents selected from SO₃H, C₁-C₁₀-alkoxy, C₁-C₁₀-alkyl and C₁-C₁₀-alkyl substituted by COOH.

According to a further particular embodiment, m is 2 and R¹¹, R¹², R¹³, R¹⁴, R²¹, R²², R²³ and R²⁴ are as defined above.

According to a special embodiment, m is 2 and R¹¹, R¹², R¹³, R¹⁴, R²¹, R²², R²³ and R²⁴ are selected from the group consisting of hydrogen, C₁-C₃₀-alkoxy, C₁-C₃₀-alkylsulfanyl, phenyloxy and phenylthio, where the two last mentioned radicals are unsubstituted or carry 1, 2 or 3 substituents selected from SO₃H, C₁-C₁₀-alkoxy, C₁-C₁₀-alkyl and C₁-C₁₀-alkyl substituted by COOH.

Especially preferred is the group of embodiments 2, wherein m is 2 and R¹¹, R¹², R¹³, R¹⁴, R²¹, R²², R²³ and R²⁴ are each hydrogen.

Likewise, especially preferred is the group of embodiments 2, wherein m is 2 and 2 or 4 of the radicals R¹¹, R¹², R¹³, R¹⁴, R²¹, R²², R²³ and R²⁴ are selected from phenyloxy which is unsubstituted or substituted by 1, 2 or 3 substituents selected from SO₃H, C₁-C₁₀-alkoxy and C₁-C₁₀-alkyl which is unsubstituted or substituted by COOH and the remaining radicals R¹¹, R¹², R¹³, R¹⁴, R²¹, R²², R²³ and R²⁴ are each hydrogen.

In particular, 2 or 4 of the the R¹², R¹⁴, R²¹ and R²³ radicals are phenyloxy which is unsubstituted or substituted by 1, 2 or 3 substituents selected from SO₃H, C₁-C₁₀-alkoxy and C₁-C₁₀-alkyl which is unsubstituted or substituted by COOH and the remaining radicals R¹¹, R¹², R¹³, R¹⁴, R²¹, R²², R²³ and R²⁸ are each hydrogen.

Some particularly preferred compounds of the second group of embodiments are specified below:

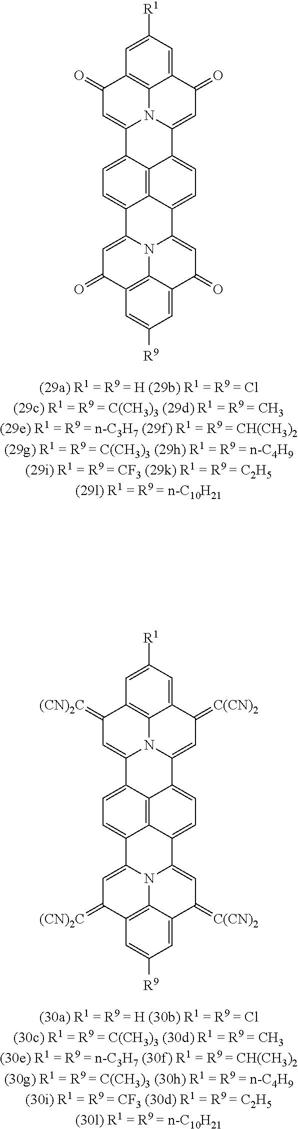

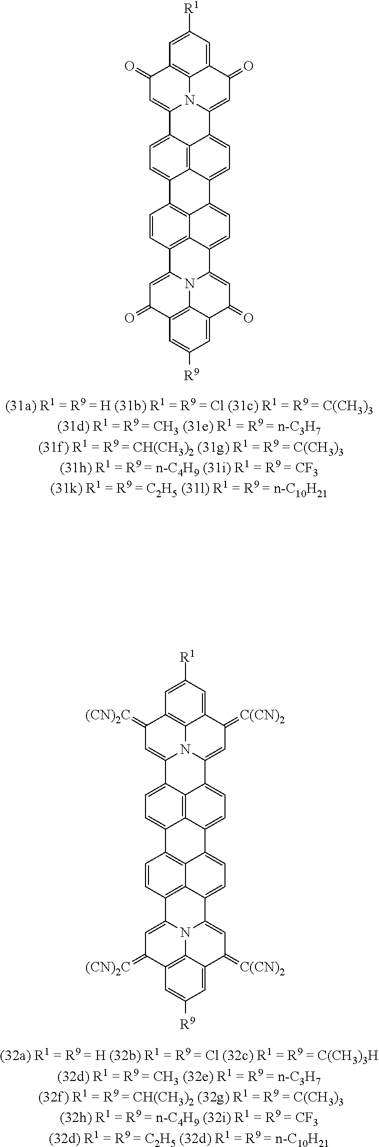

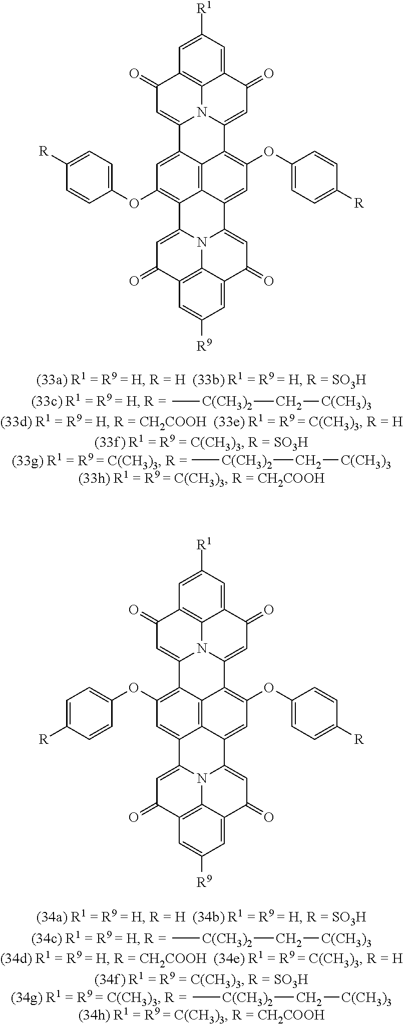

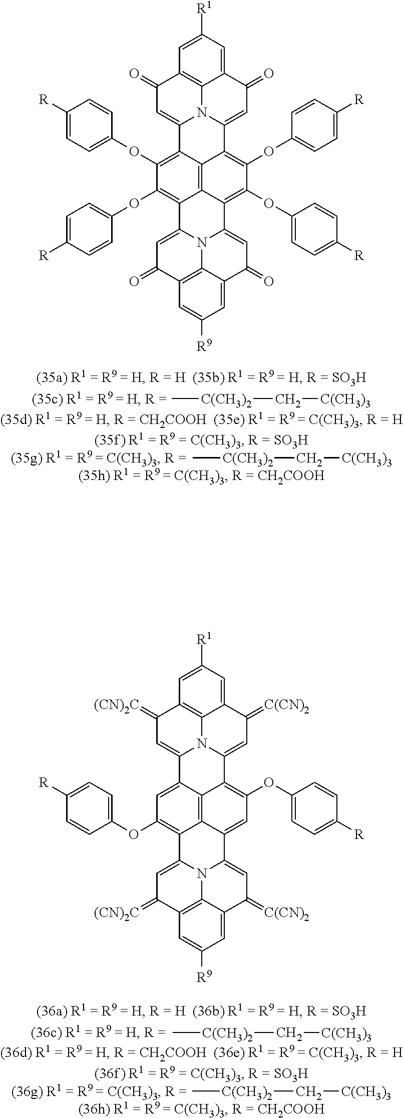

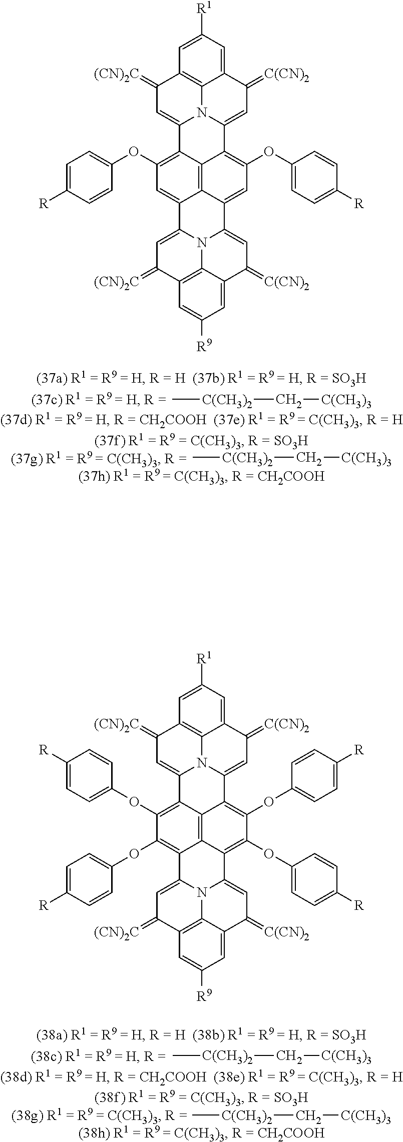

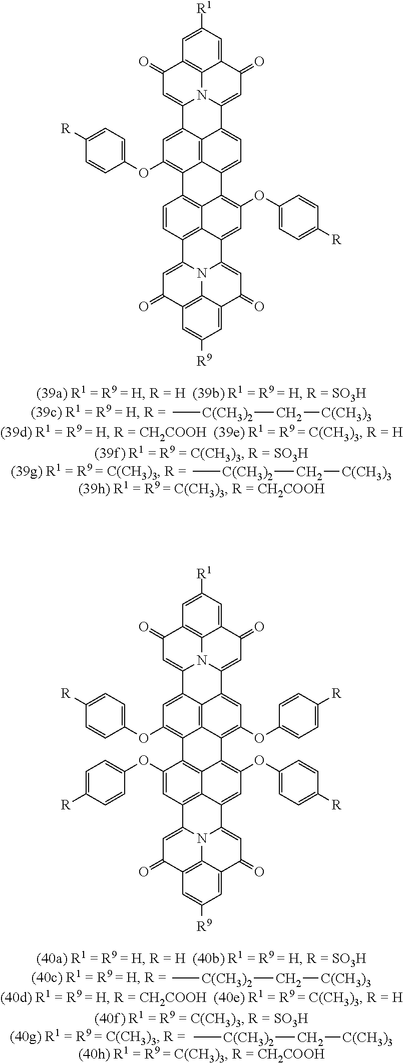

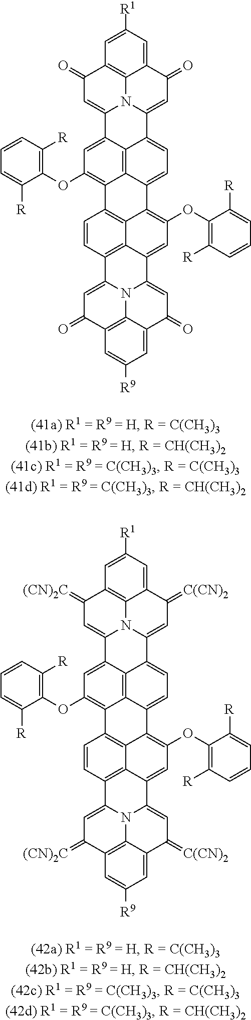

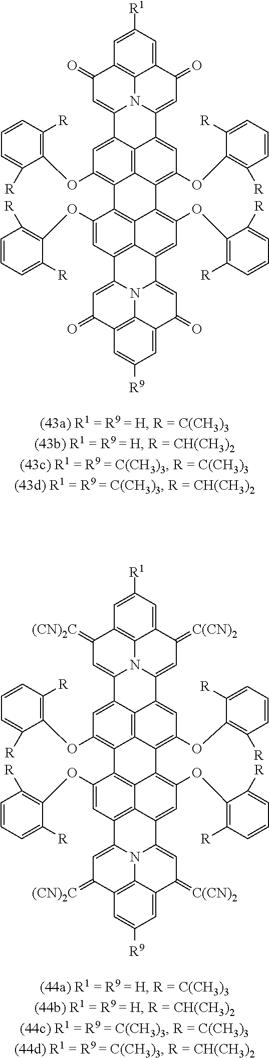

### Group of Embodiments 3

It is also preferred that in the compounds of the general formula (I) group A is selected from groups of the general formula (II.6),

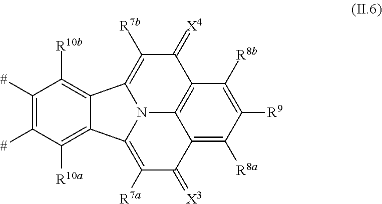

in which
X³ and X⁴ are independently of one another selected from O and C(CN)₂;
R^{7a}, R^{7b}, R^{8a}, R^{8b}, R⁹ and, if present, each R^{m1}, R^{m2}, R^{m3} and R^{m4}, are independently of one another selected from hydrogen, F, Cl, Br, I, CN, hydroxy, mercapto, nitro, cyanato, thiocyanato, formyl, acyl, carboxy, carboxylate, alkylcarbonyloxy, carbamoyl, alkylaminocarbonyl, dialkylaminocarbonyl, sulfo, sulfonate, sulfoamino, sulfamoyl, alkylsulfonyl, arylsulfonyl, amidino, NE¹E², where E¹ and E² are each independently selected from hydrogen, alkyl, cycloalkyl, heterocycloalkyl, aryl and hetaryl,
   in each case unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, heterocycloalkyl, heterocycloalkoxy, heterocycloalkylthio, (monoheterocycloalkyl)amino, (diheterocycloalkyl)amino, aryl, aryloxy, arylthio, (monoaryl)amino, (diaryl)amino, hetaryl, hetaryloxy, hetarylthio, (monohetaryl)amino and (dihetaryl)amino;
R^{10a} and R^{10b}, if present, are independently of one another selected from are independently of one another selected from hydrogen, F, Cl, Br, I, CN, hydroxy, mercapto, nitro, cyanato, thiocyanato, formyl, acyl, carboxy, carboxylate, alkylcarbonyloxy, carbamoyl, alkylaminocarbonyl, dialkylaminocarbonyl, sulfo, sulfonate, sulfoamino, sulfamoyl, alkylsulfonyl, arylsulfonyl, amidino, NE¹E², where E¹ and E² are each independently selected from hydrogen, alkyl, cycloalkyl, heterocycloalkyl, aryl and hetaryl,
   in each case unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, heterocycloalkyl, heterocycloalkoxy, heterocycloalkylthio, (monoheterocycloalkyl)amino, (diheterocycloalkyl)amino, aryl, aryloxy, arylthio, (monoaryl)amino, (diaryl)amino, hetaryl, hetaryloxy, hetarylthio, (monohetaryl)amino and (dihetaryl)amino.

The compounds of the general formula (I), wherein group A is selected from the group of the general formula (II.6), are denoted in the following also as “group of embodiments 3”. The compounds of the general formula (I), wherein group A is a group of formula (II.6) are also referred to as compounds of the formula (I.D).

In the compounds of the formula (I) according to the group of embodiments 3, the R¹ and R⁹ radicals may have identical or different definitions. In a preferred embodiment, the R¹ and R⁹ radicals have identical definitions. In the compounds of the formula (I) according to the group of embodiments 3, the R^{2a} and R^{2b} radicals may have identical or different definitions. In a preferred embodiment, the R^{2a} and R^{2b} radicals have identical definitions. In the compounds of the formula (I) according to embodiment 3, the R^{3a} and R^{3b} radicals may have identical or different definitions. In a preferred embodiment, the R^{3a} and R^{3b} radicals have identical definitions. In the compounds of the formula (I) according to embodiment 3, the R^{7a} and R^{7b} radicals may have identical or different definitions. In a preferred embodiment, the R^{7a} and R^{7b} radicals have identical definitions. In the compounds of the formula (I) according to embodiment 3, the R^{8a} and R^{8b} radicals may have identical or different definitions. In a preferred embodiment, the R^{8a} and R^{8b} radicals have identical definitions. In the compounds of the formula (I) according to embodiment 3, the R^{10a} and R^{10b} radicals may have identical or different definitions. In a preferred embodiment, the R^{10a} and R^{10b} radicals have identical definitions.

Preferably, in the group of embodiments 3, X¹, X², X³ and X⁴ are all O or all C(CN)₂.

In the group of embodiments 3, preferably R¹ has one of the preferred meanings mentioned above for the group of embodiments 1.

In the group of embodiments 3, preferably R^{2a}, R^{2b}, R^{3a} and R^{3b} have one of the preferred meanings mentioned above for the group of embodiments 1. In particular R^{2a}, R^{2b}, R^{3a} and R^{3b} are each hydrogen.

In the group of embodiments 3, preferably R^{10a} and R^{10b} are selected from hydrogen, fluorine, chlorine, cyano, C₁-C₃₀-alkyl and aryl which is unsubstituted or carries one, two or more than two C₁-C₁₀-alkyl radicals.

In the group of embodiments 3, preferably the radicals R¹, R^{2a}, R^{2b}, R^{3a}, R^{3b}, R^{7a}, R^{7b}, R^{8a}, R^{8b} and R⁹ are independently of one another selected from hydrogen, chlorine, bromine, linear C₁-C₃₀-alkyl, branched C₃-C₃₀-alkyl, perfluoro-C₁-C₃₀-alkyl, 1H,1H-perfluoro-C₂-C₃₀-alkyl, 1H,1H,2H,2H-perfluoro-C₃-C₃₀-alkyl, a radical of the formula G.1, a radical of the formula G.2 and a radical of the formula G.3

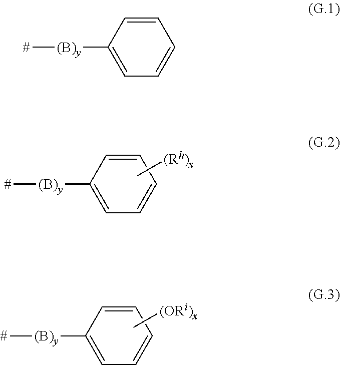

where
# represents the bonding side to the remainder of the molecule,
B where present, is selected from O, S and a C₁-C₁₀-alkylene group which may be interrupted by one or more nonadjacent groups which are selected from O and S,
y is 0 or 1,
R^{h} is independently of one another selected from C₁-C₃₀-alkyl, C₁-C₃₀-fluoroalkyl, fluorine, chlorine, bromine, NE³E⁴, nitro and cyano, where E³ and E⁴, independently of one another, are hydrogen, alkyl, cycloalkyl, heterocycloalkyl, aryl or hetaryl,
Rⁱ is independently of one another selected from C₁-C₃₀-alkyl,
x in formulae G.2 and G.3 is 1, 2, 3, 4 or 5.

In the group of embodiments 3, the radicals R¹ and R⁹ are preferably independently of one another selected from hydrogen, chlorine, bromine, C₁-C₃₀-alkyl and C₁-C₃₀-haloalkyl.

In particular, R¹ and R⁹ are selected from
hydrogen,
chlorine, bromine,
linear C₁-C₃₀-alkyl,
branched C₃-C₃₀-alkyl,
perfluoro-C₁-C₃₀-alkyl,
1H,1H-perfluoro-C₂-C₃₀-alkyl, and
1H,1H,2H,2H-perfluoro-C₃-C₃₀-alkyl.

In particular, R¹ and R⁹ are selected from
hydrogen,
chlorine, bromine,
methyl, ethyl, n-propyl, n-butyl, n-pentyl, n-hexyl, n-heptyl, n-octyl, n-nonyl, n-decyl, n-undecyl, n-dodecyl, n-tridecyl, n-tetradecyl, n-hexadecyl, n-octadecyl, n-eicosyl,
branched C₃-C₃₀-alkyl, selected from radicals of the general formulae (III.1) and (III.2)

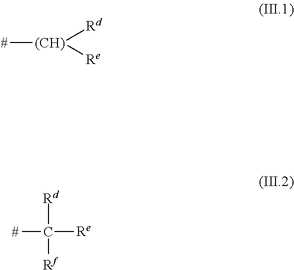

in which
# is a bonding site to the remainder of the molecule, and
in the formula (III.1) R^{d} and R^{e} are independently selected from C₁- to C₂₈-alkyl, where the sum of the carbon atoms of the R^{d} and R^{e} radicals is an integer from 2 to 29,
in the formula (III.2) R^{d}, R^{e} and R^{f} are independently selected from C₁- to C₂₇-alkyl, where the sum of the carbon atoms of the R^{d}, R^{e} and R^{f} radicals is an integer from 3 to 29,
CF₃, C₂F₅, n-C₃F₇, n-C₄F₉, n-C₅F₁₁, n-C₆F₁₃, CF(CF₃)₂, C(CF₃)₃, CF₂CF(CF₃)₂, CF(CF₃)(C₂F₅),
CH₂—CF₃, CH₂—C₂F₅, CH₂-(n-C₃F₇), CH₂-(n-C₄F₉), CH₂-(n-C₅F₁₁), CH₂-(n-C₆F₁₃), CH₂—CF(CF₃)₂, CH₂—C(CF₃)₃, CH₂—CF₂CF(CF₃)₂, CH₂—CF(CF₃)(C₂F₅),
CH₂—CH₂—CF₃, CH₂—CH₂—C₂F₅, CH₂—CH₂-(n-C₃F₇), CH₂—CH₂-(n-C₄F₉), CH₂—CH₂-(n- C₅F₁₁), CH₂—CH₂-(n-C₆F₁₃), CH₂—CH₂—CF(CF₃)₂, CH₂—CH₂—C(CF₃)₃, CH₂—CH₂—CF₂CF(CF₃)₂ and CH₂—CH₂—CF(CF₃)(C₂F₅).

In a more preferred embodiment, the radicals R¹ and R⁹ are independently of one another selected from hydrogen, chlorine, bromine, linear C₁-C₁₀-alkyl, a radical of the formula (III.1) or a radical of the formula (III.2). In the context of the formulae (III.1) and (III.2), the R^{d}, R^{e} and R^{f} radicals are independently selected from C₁- to C₁₂-alkyl, especially C₁- to C₈-alkyl.

Examples of preferred radicals of the formula (III.1) are:

1-methylethyl, 1-methylpropyl, 1-methylbutyl, 1-methylpentyl, 1-methylhexyl, 1-methylheptyl, 1-methyloctyl, 1-ethylpropyl, 1-ethylbutyl, 1-ethylpentyl, 1-ethylhexyl, 1-ethylheptyl, 1-ethyloctyl, 1-propylbutyl, 1-propylpentyl, 1-propylhexyl, 1-propylheptyl, 1-propyloctyl, 1-butylpentyl, 1-butylhexyl, 1-butylheptyl, 1-butyloctyl, 1-pentylhexyl, 1-pentylheptyl, 1-pentyloctyl, 1-hexylheptyl, 1-hexyloctyl, 1-heptyloctyl.

A particularly preferred radical of the formula (III.2) is tert.-butyl.

More preferably, the radicals R¹ and R⁹ are independently of one another selected from from hydrogen, chlorine, bromine, linear C₁-C₁₀-alkyl and branched C₃-C₁₀-alkyl.

Especially, R¹ and R⁹ have the same meaning and are selected from hydrogen, chlorine, bromine, linear C₁-C₁₀-alkyl and branched C₃-C₁₀-alkyl. Particularly preferred linear C₁-C₁₀-alkyl radicals are n-propyl or n-decyl. Particularly preferred branched C₃-C₁₀-alkyl radicals are isopropyl and tert.-butyl.

In the group of embodiments 3, the radicals R^{2a}, R^{2b}, R^{8a} and R^{8b} may have identical or different definitions. Preferably, the radicals R^{2a}, R^{2b}, R^{8a} and R^{8b} have identical definitions. In particular, R^{2a}, R^{2b}, R^{8a} and R^{8b} are each hydrogen.

In the group of embodiments 3, the radicals R^{3a}, R^{3b}, R^{7a} and R^{7b} may have identical or different definitions. Preferably, the radicals R^{3a}, R^{3b}, R^{7a} and R^{7b} have identical definitions. In particular, R^{3a}, R^{3b}, R^{7a} and R^{7b} are each hydrogen.

In a special embodiment, R^{10a} and R^{10b} are both hydrogen.

Some particularly preferred compounds of the third group of embodiments are specified below:

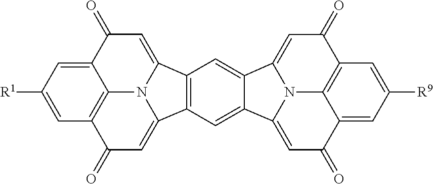

(45a) R¹ = R⁹ = H
(45b) R¹ = R⁹ = CH₃
(45c) R¹ = R⁹ = C₂H₅
(45d) R¹ = R⁹ = CH₂CH₂CH₃
(45e) R¹ = R⁹ = CH(CH₃)₂
(45f) R¹ = R⁹ = C(CH₃)₃
(45g) R¹ = R⁹ = n-C₄H₉
(45h) R¹ = R⁹ = CF₃
(45i) R¹ = R⁹ = n-C₁₀H₂₁

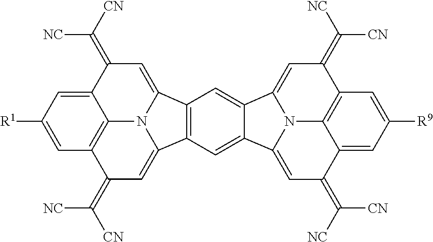

(46a) R¹ = R⁹ = H
(46b) R¹ = R⁹ = CH₃
(46c) R¹ = R⁹ = C₂H₅
(46d) R¹ = R⁹ = CH₂CH₂CH₃
(46e) R¹ = R⁹ = CH(CH₃)₂
(46f) R¹ = R⁹ = C(CH₃)₃
(46g) R¹ = R⁹ = n-C₄H₉
(46h) R¹ = R⁹ = CF₃
(46i) R¹ = R⁹ = n-C₁₀H₂₁

A further object of the invention is a process for the preparation of a compound of the formula I.A,

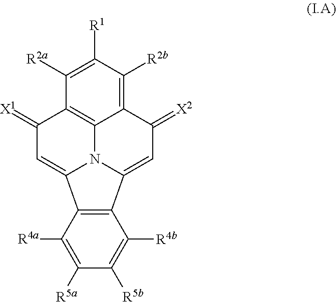

wherein
X¹ and X² are independently of one another selected from O and C(CN)₂,
R¹, R^{2a}, R^{2b}, R^{4a}, R^{4b}, R^{5a} and R^{5b} are independently of one another selected from hydrogen, F, Cl, Br, I, CN, hydroxy, mercapto, nitro, cyanato, thiocyanato, formyl, acyl, carboxy, carboxylate, alkylcarbonyloxy, carbamoyl, alkylaminocarbonyl, dialkylaminocarbonyl, sulfo, sulfonate, sulfoamino, sulfamoyl, alkylsulfonyl, arylsulfonyl, amidino, NE¹E², where E¹ and E² are each independently selected from hydrogen, alkyl, cycloalkyl, heterocycloalkyl, aryl or hetaryl,
   in each case unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, heterocycloalkyl, heterocycloalkoxy, heterocycloalkylthio, (monoheterocycloalkyl)amino, (diheterocycloalkyl)amino, aryl, aryloxy, arylthio, (monoaryl)amino, (diaryl)amino, hetaryl, hetaryloxy, hetarylthio, (monohetaryl)amino and (dihetaryl)amino,
      comprising
i.a) reacting a phthalic anhydride compound (A) and a 2,6-dibromoaniline compound (B1) to obtain an imide compound of the formula (C)

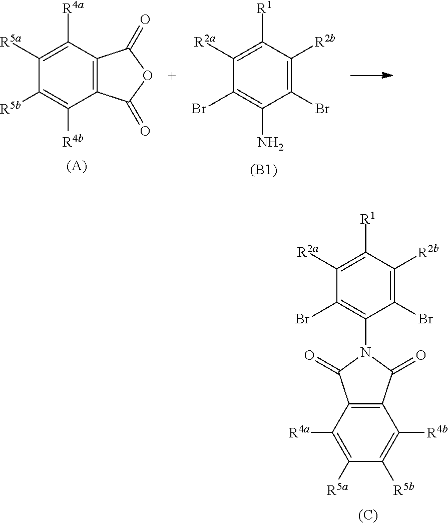

ii.a) reacting the compound of the formula (C) with tributyl(1-ethoxy-1-ethenyl)-stannane in the presence of a Pd catalyst to obtain a compound (D)

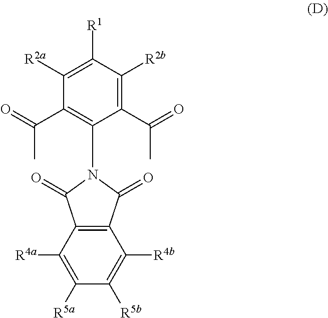

iii.a) subjecting the compound of the formula (D) to a condensation reaction to obtain a compound (I.A1)

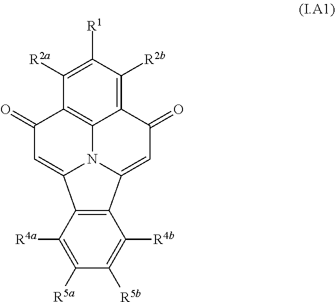

iv.a) optionally reacting the compound of the formula (I.A1) with malononitrile to obtain a compound (I.A2)

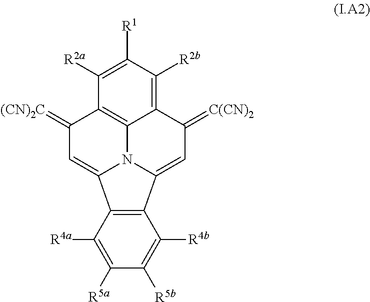

### Step i.a)

Phthalic anhydride and substituted phthalic anhydrides (A) are commercially available or can be synthesized by processes known in the art. Compounds of formula B1 such as 2,6-dibromoaniniline (CAS 608-30-0), 2,6-dibromo-4-methylaniline (CAS 6968-24-7), 2,6-dibromo-4-n-propylaniline(CAS 10546-64-2), 2,6-dibromo-4-isopropylaniline (CAS 10546-65-3) 2,6-dibromo-4-tert-butylaniline (=2,6-dibromo-4-(1,1-dimethylethyl)-benzenamine, CAS No. 10546-67-5) and 2,6-dibromo-4-(trifluoromethyl)aniline (CAS 72678-19-4) are also readily available. Derivatives can be synthesized by the skilled artisan using known methods. The synthesis of the phthalimides (C) can be performed by direct dehydrative condensation of the phthalic anhydride compound (A) with the primary amine (B1).

A preferred solvent for the reaction in step i.a) is acetic acid.

The reaction temperature in step i.a) is generally in a range of from 50 to 250° C., preferably 80 to 150° C. In a preferred embodiment the reaction is performed in acetic acid at reflux temperature at ambient pressure.

### Step ii.a)

In step ii.a) the compound of the formula (C) is reacted with tributyl(1-ethoxy-1-ethenyl)stannane in the presence of a Pd catalyst in a Stille type coupling reaction.

Tributyl(1-ethoxy-1-ethenyl)stannane (=tributyl(1-ethoxyvinyl)tin) is commercially available, e.g. from Sigma Aldrich.

The coupling reaction is preferably effected in the presence of a palladium catalyst under reaction conditions known per se for Stille type coupling reactions. Suitable catalysts are especially palladium-catalysts, for example Pd₂(dba)₃/BINAP, Pd₂(dba)₃/Tol-BINAP, Pd(OAc)₂, Pd₂(dba)₃, Pd₂(dba)₃/2-(dicyclohexylphosphino)-biphenyl, Pd(OAc)₂/2-(dicyclohexylphosphino)-biphenyl, Pd(OAc)₂/2-(di-t-butyl-phosphino)-2′-methylbiphenyl, Pd(dba)₂/DTPE, Pd(dba)₂/DPPF, Pd(OAc)₂/Xantphos, Pd(OAc)₂/n-butylbis(1-adamantyl)-phosphine, Pd(dba)₂/n-butylbis(1-adamantyl)-phosphine, Pd(OAc)₂/PPh₃, Pd(OAc)₂/(4-XC₆H₄)₃P, Pd₂(dba)₃/Xantphos, Pd(OAc)₂/2-(Dicyclohexylphosphino)-2′-methylbiphenyl, Pd(OAc)₂/DPPP, PdCl₂(Ph₃P)₂, PdCl₂-[(o-Tol)₃]2, Pd(Ph₃P)₄, Pd(OAc)₂/P(t-Bu)₃, Pd₂(dba)₃/CHCl₃/BINAP, and combinations thereof. The amount of catalyst is typically 1 to 20 mol %, especially 1.5 to 5 mol %, based on the tributyl(1-ethoxy-1-ethenyl)stannane used.

The process in step ii.a) can be carried out in a suitable solvent which is inert under the respective reaction conditions. Solvents which are generally suitable are, for example, aromatics such as toluene and xylenes, hydrocarbons or mixtures of hydrocarbons such as cyclohexane, ethers such as tert-butyl methyl ether, 1,4-dioxane and tetrahydrofuran, dimethylformamide, dimethylsulfoxide, ionic liquids, etc. A particularly preferred solvent is dioxane.

The reaction temperature is generally 20 to 180° C., preferably 20 to 120° C.

The reaction in step ii.a) can be carried out in the presence of an inert gas, e.g. nitrogen, argon, etc.

### Step iii.a)

The aldol condensation in step iii.a) is preferably performed in the presence of a base. Preferably the base is selected from strong sterically hindered bases. Suitable bases are e.g. 1,8-diazabicyclo[5.4.0]undec-7-ene (DBU), 1,5-diazobicyclo[4.3.0]non-5-en (DBN), 1,4-diazabicylo[2.2.2]octane (DABCO), etc.

Suitable solvents are e.g. polar aprotic solvents, such as acetonitrile, nitrogen-containing heterocycles, N,N-disubstituted aliphatic carboxamides (preferably N,N-di(C₁-C₄-alkyl)(C₁-C₄)carboxamides) and N-alkyllactams such as dimethylformamide, diethylformamide, dimethylacetamide, dimethylbutyramide and N-methylpyrrolidone; tetrahydrofuran, 1,4-dioxane.

The reaction temperature is generally from 10 to 100° C., preferably from 15 to 50° C.

In a further preferred embodiment, the base is imidazole. In this case, the imidazole act as base and also serves as solvent. The reaction is usually carried out at reflux temperature.

In a preferred embodiment, the reaction in reaction is performed under a protective gas atmosphere, for example nitrogen or argon.

### Step iv.a)

The reaction in step iv.a) can be regarded as a Knoevenagel condensation reaction of the compound of the formula (I.A1) with malononitrile.

In one embodiment the reaction is performed in the presence of an acid as catalyst. A preferred acid is a mixture of glacial acetic acid and acetic anhydride.

In another embodiment the reaction is performed in the presence of a Lewis acid, preferably TiCl₄ and pyridine.

A further object of the invention is a process for the preparation of a compound of the formula I.B,

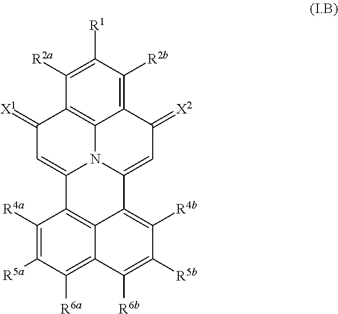

wherein
X¹ and X² are independently of one another selected from O and C(CN)₂,
R¹, R^{2a}, R^{2b}, R^{4a}, R^{4b}, R^{5a}, R^{5b}, R^{6a} and R^{6b} are independently of one another selected from hydrogen, F, Cl, Br, I, CN, hydroxy, mercapto, nitro, cyanato, thiocyanato, formyl, acyl, carboxy, carboxylate, alkylcarbonyloxy, carbamoyl, alkylaminocarbonyl, dialkylaminocarbonyl, sulfo, sulfonate, sulfoamino, sulfamoyl, alkylsulfonyl, arylsulfonyl, amidino, NE¹E², where E¹ and E² are each independently selected from hydrogen, alkyl, cycloalkyl, heterocycloalkyl, aryl or hetaryl,
   in each case unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, heterocycloalkyl, heterocycloalkoxy, heterocycloalkylthio, (monoheterocycloalkyl)amino, (diheterocycloalkyl)amino, aryl, aryloxy, arylthio, (monoaryl)amino, (diaryl)amino, hetaryl, hetaryloxy, hetarylthio, (monohetaryl)amino and (dihetaryl)amino,
      comprising
i.b) reacting a 2,6-dibromoaniline (B1) with tributyl(1-ethoxy-1-ethenyl)stannane in the presence of a Pd catalyst to obtain a compound (E1)

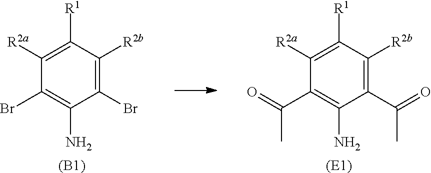

ii.b) reacting the compound of the formula (E1) with a naphthalene monoanhydride (F) to obtain a compound (I.B1)

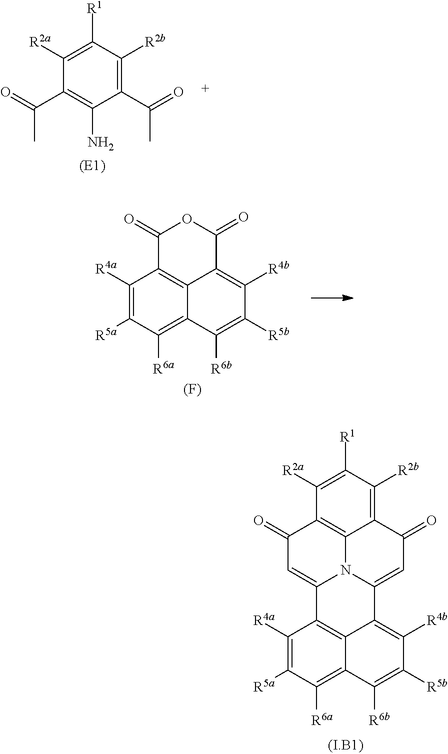

iii.b) optionally reacting the compound of the formula (I.B1) with malononitrile to obtain a compound (I.B2)

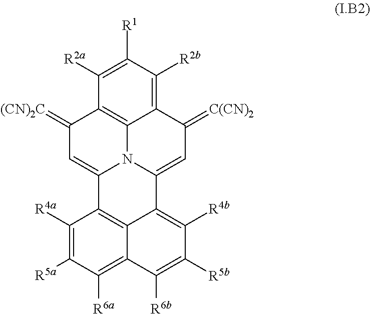

### Step i.b)

In step i.b), 2,6-dibromoaniline of the formula B1 is reacted with tributyl(1-ethoxy-1-ethenyl)-stannane in the presence of a palladium catalyst in a Stille coupling reaction. The coupling reaction is preferably effected under reaction conditions known per se for Stille type coupling reactions. With regard to suitable and preferred reaction conditions, reference is made to the reaction conditions of step ii.a) described above.

### Step ii.b)

Naphthalic anhydrides (F) are commercially available or can be synthesized by processes known in the art. Derivatives can be synthesized by the skilled artisan using known methods. The synthesis of the compound (I.B1) can be performed by direct dehydrative condensation.

The reaction temperature in step ii.b) is generally in a range of from 80 to 250° C., preferably 100 to 200° C.

The reaction is preferably performed in the presence of catalytic amounts of zinc acetate and a base. Suitable bases are e.g. basic nitrogen containing heterocycles, which may also serve as solvent for the reaction. A preferred base is imidazole.

### Step ii.c)

The reaction in step ii.c) can be regarded as a Knowvenagel condensation reaction of the compound of formula (I.B1) with malonitrile. With regard to suitable and preferred reaction conditions, reference is made to the reaction conditions of step iv.a) described above.

A further object of the invention is a process for the preparation of a compound of the formula I.C,

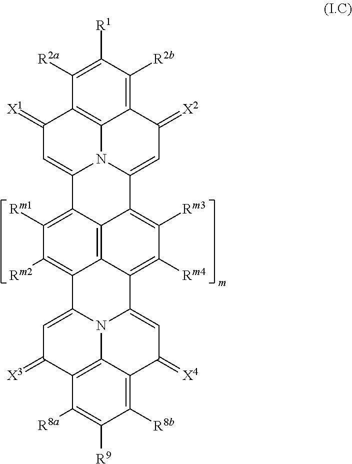

wherein
m is 1, 2, 3 or 4,
X¹, X², X³ and X⁴ are independently of one another selected from O and C(CN)₂,
R¹, R^{2a}, R^{2b}, R^{8a}, R^{8b}, R⁹ and each R^{m1}, R^{m2}, R^{m3} and R^{m4} are independently of one another selected from hydrogen, F, C, Br, I, CN, hydroxy, mercapto, nitro, cyanato, thiocyanato, formyl, acyl, carboxy, carboxylate, alkylcarbonyloxy, carbamoyl, alkylaminocarbonyl, dialkylaminocarbonyl, sulfo, sulfonate, sulfoamino, sulfamoyl, alkylsulfonyl, arylsulfonyl, amidino, NE¹E², where E¹ and E² are each independently selected from hydrogen, alkyl, cycloalkyl, heterocycloalkyl, aryl or hetaryl,
   in each case unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, heterocycloalkyl, heterocycloalkoxy, heterocycloalkylthio, (monoheterocycloalkyl)amino, (diheterocycloalkyl)amino, aryl, aryloxy, arylthio, (monoaryl)amino, (diaryl)amino, hetaryl, hetaryloxy, hetarylthio, (monohetaryl)amino and (dihetaryl)amino,
      comprising
i.c) reacting a rylene tetracarboxylic acid bisanhydride (K)

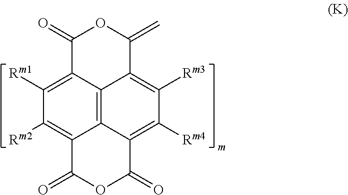

with a 2,6-dibromoaniline (B1)

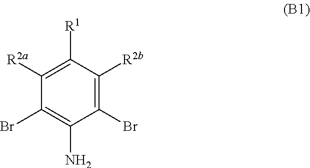

and optionally a different 2,6-dibromoaniline (B2)

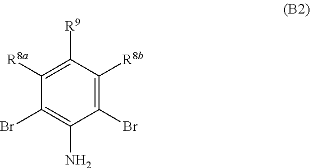

to obtain an imide compound of the formula (L)

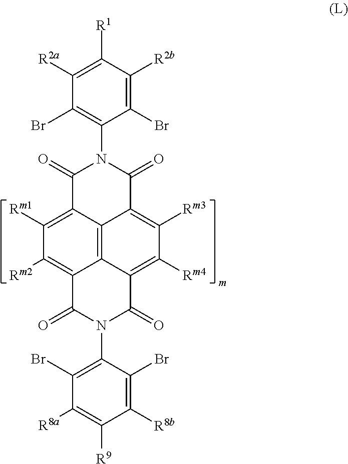

ii.c) reacting the compound of the formula (L) with tributyl(1-ethoxy-1-ethenyl)-stannane in the presence of a Pd catalyst to obtain a compound (M)

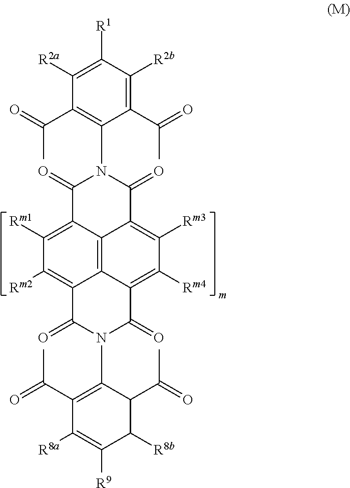

iii.c) subjecting the compound of the formula (M) to a condensation reaction to obtain a compound (I.C1)

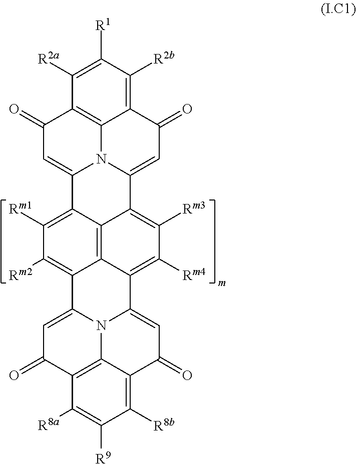

iv.c) optionally reacting the compound of the formula (I.C1) with malononitrile to obtain a compound (I.C2)

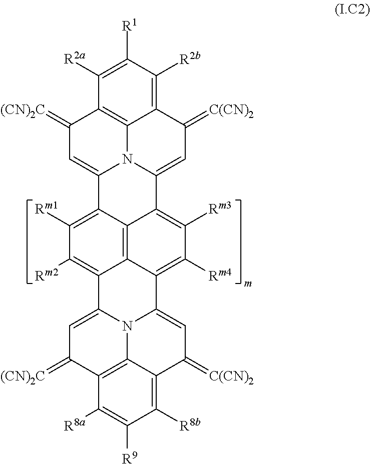

### Step i.c)

Rylene tetracarboxylic anhydrides (K) are commercially available or can be synthesized by processes known in the art. As mentioned before, 2,6-dibromo-4-tert-butylaniline (B) (=2,6-dibromo-4-(1,1-dimethylethyl)-benzenamine, CAS No. 10546-67-5) is also readily available. Derivatives can be synthesized by the skilled artisan using known methods. The synthesis of the phthalimides (L) can be performed by direct dehydrative condensation of the rylene tetracarboxylic anhydride (K) with the compound (B1) and optionally a different compound (B2).

The reaction temperature in step i.c) is generally in a range of from 50 to 250° C., preferably 80 to 200° C.

The reaction is preferably performed in the presence of catalytic amounts of zinc acetate and a base. Suitable bases are e.g. basic nitrogen containing heterocycles, which may also serve as solvent for the reaction. A preferred base is imidazole.

### Step ii.c)

In step ii.c) the compound of the formula (L) is reacted with tributyl(1-ethoxy-1-ethenyl)-stannane in the presence of a Pd catalyst in a Stille type coupling reaction.

The coupling reaction is preferably effected in the presence of a palladium catalyst under reaction conditions known per se for Stille type coupling reactions. With regard to suitable and preferred reaction conditions of step ii.c) reference is made to the reaction conditions of step ii.a) described above.

### Step iii.c)

The aldol condensation in step iii.c) is preferably performed in the presence of a base.

Suitable bases are e.g. basic nitrogen containing heterocycles, which may also serve as solvent for the reaction. A preferred base is imidazole. Suitable bases are also strong sterically hindered bases, e.g. 1,8-diazabicyclo[5.4.0]undec-7-ene (DBU), 1,5-diazobicyclo[4.3.0]non-5-en (DBN), 1,4-diazabicylo[2.2.2]octane (DABCO), etc.

The reaction temperature is generally from 10 to 180° C., preferably from 20 to 150° C.

In a preferred embodiment, the reaction in reaction is performed under a protective gas atmosphere, for example nitrogen or argon.

### Step iv.c)

The reaction in step iv.c) can be regarded as a Knoevenagel condensation reaction of the compound of the formula (I.C1) with malononitrile.

In one embodiment the reaction is performed in the presence of an acid as catalyst. A preferred acid is a mixture of glacial acetic acid and acetic anhydride.

A further object of the invention is a process for the preparation of a compound of the formula I.C,

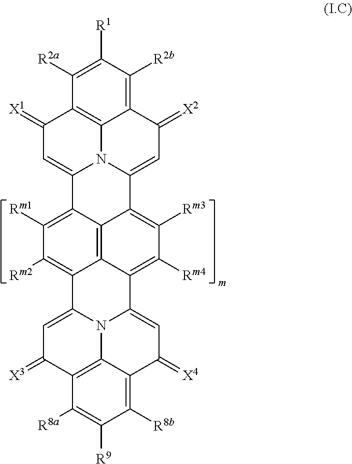

wherein
m is 1, 2, 3 or 4,
X¹, X², X³ and X⁴ are independently of one another selected from O and C(CN)₂,
R¹, R^{2a}, R^{2b}, R^{8a}, R^{8b}, R⁹ and each R^{m1}, R^{m2}, R^{m3} and R^{m4} are independently of one another selected from hydrogen, F, Cl, Br, I, CN, hydroxy, mercapto, nitro, cyanato, thiocyanato, formyl, acyl, carboxy, carboxylate, alkylcarbonyloxy, carbamoyl, alkylaminocarbonyl, dialkylaminocarbonyl, sulfo, sulfonate, sulfoamino, sulfamoyl, alkylsulfonyl, arylsulfonyl, amidino, NE¹E², where E¹ and E² are each independently selected from hydrogen, alkyl, cycloalkyl, heterocycloalkyl, aryl or hetaryl,
   in each case unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, heterocycloalkyl, heterocycloalkoxy, heterocycloalkylthio, (monoheterocycloalkyl)amino, (diheterocycloalkyl)amino, aryl, aryloxy, arylthio, (monoaryl)amino, (diaryl)amino, hetaryl, hetaryloxy, hetarylthio, (monohetaryl)amino and (dihetaryl)amino,
      comprising
i.d) reacting a rylene tetracarboxylic acid bisanhydride (K)

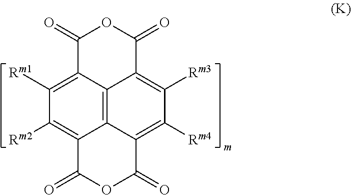

with a compound of the formula (E1)

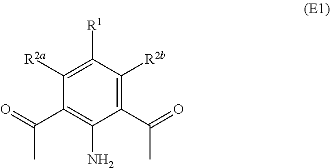

and optionally with a different compound (E2)

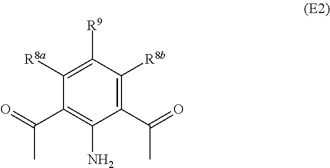

to obtain a compound (I.C1)

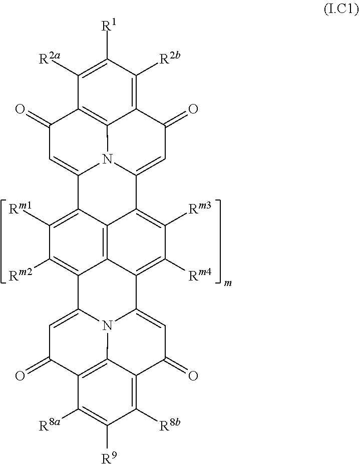

ii.d) optionally reacting the compound of the formula (I.C1) with malononitrile to obtain a compound (I.C2)

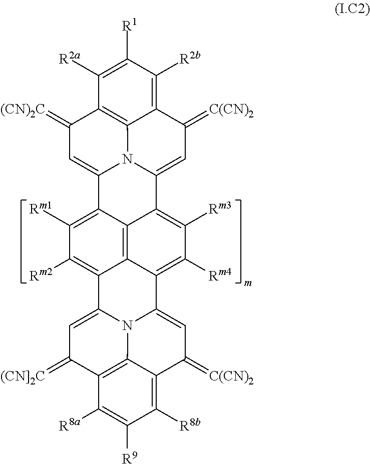

### Step i.d)

Rylene tetracarboxylic anhydrides (K) can be subjected to a one pot reaction comprising the imidation with a compound of the formula (E1) and optionally with a different compound (E2) and the subsequent condensation reaction.

The preparation of compounds (E1) and (E2) can be performed by reacting a corresponding 2,6-dibromoaniline with tributyl(l-ethoxy-1-ethenyl)stannane in the presence of a Pd catalyst as described in step i.b) above.

The reaction of the rylene tetracarboxylic anhydrides (K) with a compound of the formula (E1) and optionally with a different compound (E2) is preferably performed at a reaction temperature in a range of from 50 to 250° C., preferably 80 to 200° C.

The reaction is preferably performed in the presence of catalytic amounts of zinc acetate and a base. Suitable bases are e.g. basic nitrogen containing heterocycles, which may also serve as solvent for the reaction. A preferred base is quinoline.

### Step ii.d)

With regard to suitable and preferred reaction conditions of step ii.d) reference is made to the reaction conditions of step iv.c) described above.

A further object of the invention is a process for the preparation of a compound of the formula I.D

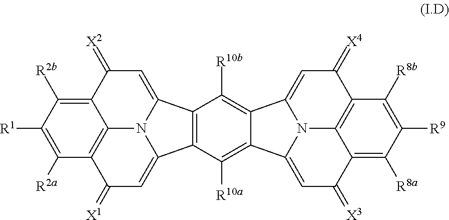

wherein
X¹, X², X³ and X⁴ are independently of one another selected from O and C(CN)₂;
R¹, R^{2a}, R^{2b}, R^{8a}, R^{8b}, R⁹ and each R^{m1}, R^{m2}, R^{m3} and R^{m4} are independently of one another selected from hydrogen, F, Cl, Br, I, CN, hydroxy, mercapto, nitro, cyanato, thiocyanato, formyl, acyl, carboxy, carboxylate, alkylcarbonyloxy, carbamoyl, alkylaminocarbonyl, dialkylaminocarbonyl, sulfo, sulfonate, sulfoamino, sulfamoyl, alkylsulfonyl, arylsulfonyl, amidino, NE¹E², where E¹ and E² are each independently selected from hydrogen, alkyl, cycloalkyl, heterocycloalkyl, aryl or hetaryl,
   in each case unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, heterocycloalkyl, heterocycloalkoxy, heterocycloalkylthio, (monoheterocycloalkyl)amino, (diheterocycloalkyl)amino, aryl, aryloxy, arylthio, (monoaryl)amino, (diaryl)amino, hetaryl, hetaryloxy, hetarylthio, (monohetaryl)amino and (dihetaryl)amino;
      comprising
i.e) reacting a pyromellitic dianhydride of the formula (N)

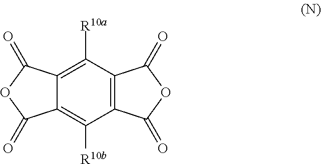

with a 2,6-dibromoaniline of the formula (B1)

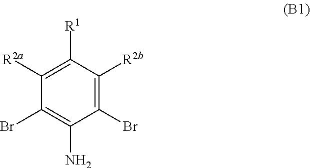

and optionally a different 2,6-dibromoaniline of the formula (B2)

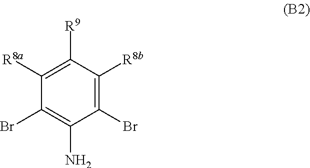

to obtain an imide compound of the formula (O)

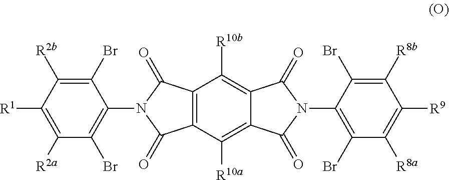

ii.e) reacting the compound of the formula (O) with tributyl(1-ethoxy-1-ethenyl)-stannane in the presence of a Pd catalyst to obtain a compound (P)

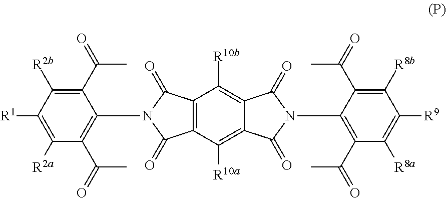

iii.e) subjecting the compound of the formula (P) to a condensation reaction to obtain a compound (I.D1)

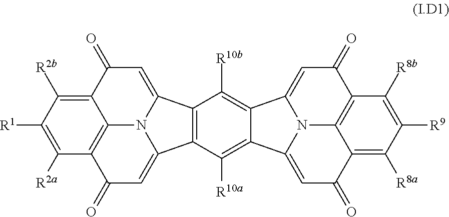

iv.e) optionally reacting the compound of the formula (I.D1) with malononitrile to obtain a compound (I.D2)

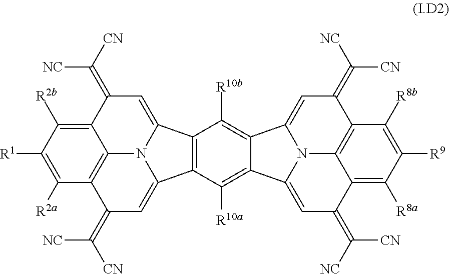

### Step i.e)

Pyromellitic dianhydride and substituted pyromellitic dianhydride (N) are commercially available or can be synthesized by processes known in the art. As mentioned before, 2,6-dibromo-aniline compounds of formula (B1) are also readily available. Derivatives can be synthesized by the skilled artisan using known methods. The synthesis of the imides (O) can be performed by direct dehydrative condensation of the dianhydride (N) with the compound (B1) and optionally a different compound (B2).

A preferred solvent for the reaction in step i.e) is acetic acid.

The reaction temperature in step i.e) is generally in a range of from 50 to 250° C., preferably 80 to 150° C. In a preferred embodiment the reaction is performed in acetic acid at reflux temperature at ambient pressure.

### Step ii.e)

In step ii.e) the compound of the formula (O) is reacted with tributyl(1-ethoxy-1-ethenyl)stannane in the presence of a Pd catalyst in a Stille type coupling reaction.

The coupling reaction is preferably effected in the presence of a palladium catalyst under reaction conditions known per se for Stille type coupling reactions. With regard to suitable and preferred reaction conditions of step ii.e) reference is made to the reaction conditions of step ii.a) described above.

### Step iii.e)

The aldol condensation in step iii.e) is preferably performed in the presence of a base.

Suitable bases are e.g. basic nitrogen containing heterocycles, which may also serve as solvent for the reaction. A preferred base is imidazole. Suitable bases are also strong sterically hindered bases, e.g. 1,8-diazabicyclo[5.4.0]undec-7-ene (DBU), 1,5-diazobicyclo[4.3.0]non-5-en (DBN), 1,4-diazabicylo[2.2.2]octane (DABCO), etc.

The reaction temperature is generally from 10 to 180° C., preferably from 20 to 150° C.

In a preferred embodiment, the reaction in reaction is performed under a protective gas atmosphere, for example nitrogen or argon.

### Step iv.e)

The reaction in step iv.e) can be regarded as a Knoevenagel condensation reaction of the compound of the formula (I.D1) with malononitrile.

In one embodiment the reaction is performed in the presence of an acid as catalyst. A preferred acid is a mixture of glacial acetic acid and acetic anhydride.

The compounds of the formula (I) are in particular suitable as organic semiconductors. They generally can function as n-type semiconductors or p-type semiconductors.

The compounds of the formula (I) are in particular suitable as organic semiconductors. They generally can function as n-type semiconductors or p-type semiconductors. In electronic devices that employ a combination of two different semiconductors, e.g. organic solar cells, it depends on the position of the energy levels (ionization potential IP and electron affinity EA) in the corresponding semiconductor material if a compound of the formula (I) acts as n-type semiconductor or as p-type semiconductor. Further, if a compound of the formula (I) acts as n-type semiconductor or as p-type semiconductors depends inter alia on the employed gate dielectric. The compounds of the formula (I) are also suitable as ambipolar semiconductors (i.e. a material which has both, hole transport properties and electron transport properties).

The compounds of the formula (I) have at least one of the following advantages over known organic semiconductor materials:
high charge transport mobility,
air stability,
high on/off ratio,
suitability to be employed in a solvent-based process.

The compounds of the formula (I) are advantageously suitable for organic field-effect transistors. They may be used, for example, for the production of integrated circuits (ICs), for which customary n-channel MOSFETs (metal oxide semiconductor field-effect transistors) have been used to date. These are then CMOS-like semiconductor units, for example for microprocessors, microcontrollers, static RAM and other digital logic circuits. For the production of semiconductor materials, the compounds of the formula (I) can be processed further by one of the following processes: printing (offset, flexographic, gravure, screenprinting, inkjet, electrophotography), evaporation, laser transfer, photolithography, drop-casting. They are especially suitable for use in displays (specifically large-surface area and/or flexible displays), RFID tags, smart labels and sensors.

The compounds of the formula (I) are advantageously suitable as electron conductors in organic field-effect transistors, organic solar cells and in organic light-emitting diodes. They are also particularly advantageous as an exciton transport material in excitonic solar cells.

Some of the compounds of the formula (I) are fluorescent and are also particularly advantageously suitable as fluorescent dyes in a display based on fluorescence conversion. Such displays comprise generally a transparent substrate, a fluorescent dye present on the substrate and a radiation source. Typical radiation sources emit blue (color by blue) or UV light (color by UV). The dyes absorb either the blue or the UV light and are used as green emitters. In these displays, for example, the red light is generated by exciting the red emitter by means of a green emitter which absorbs blue or UV light. Suitable color-by-blue displays are described, for example, in WO 98/28946. Suitable color-by-UV displays are described, for example, by W. A. Crossland, I. D. Sprigle and A. B. Davey in Photoluminescent LCDs (PL-LCD) using phosphors, Cambridge University and Screen Technology Ltd., Cambridge, UK. The compounds of the formula (I) are also particularly suitable in displays which, based on an electrophoretic effect, switch colors on and off via charged pigment dyes. Such electrophoretic displays are described, for example, in US 2004/0130776.

The invention further provides organic field-effect transistors comprising a substrate with at least one gate structure, a source electrode and a drain electrode, and at least one compound of the formula (I) as defined above as a semiconductor.

The invention further provides substrates having a plurality of organic field-effect transistors, wherein at least some of the field-effect transistors comprise at least one compound of the formula (I) as defined above.

The invention also provides semiconductor units which comprise at least one such substrate.

A specific embodiment is a substrate with a pattern (topography) of organic field-effect transistors, each transistor comprising
an organic semiconductor disposed on the substrate;
a gate structure for controlling the conductivity of the conductive channel; and
conductive source and drain electrodes at the two ends of the channel,
   the organic semiconductor consisting of at least one compound of the formula (I) or comprising a compound of the formula (I). In addition, the organic field-effect transistor generally comprises a dielectric.

A specific embodiment is a substrate with a pattern (topography) of organic field-effect transistors, each transistor comprising
an organic semiconductor disposed on a buffer layer on a substrate;
a gate structure for controlling the conductivity of the conductive channel; and
conductive source and drain electrodes at the two ends of the channel,
   the organic semiconductor consisting of at least one compound of the formula (I) or comprising a compound of the formula (I). In addition, the organic field-effect transistor generally comprises a dielectric.

As a buffer layer, any dielectric material is suitable, for example anorganic materials such LIF, AlOₓ, SiO₂ or silicium nitride or organic materials such as polyimides or polyacrylates, e.g. polymethylmethacrylate (PMMA).

A further specific embodiment is a substrate having a pattern of organic field-effect transistors, each transistor forming an integrated circuit or being part of an integrated circuit and at least some of the transistors comprising at least one compound of the formula (I).

Suitable substrates are in principle the materials known for this purpose. Suitable substrates comprise, for example, metals (preferably metals of groups 8, 9, 10 or 11 of the Periodic Table, such as Au, Ag, Cu), oxidic materials (such as glass, ceramics, SiO₂, especially quartz), semiconductors (e.g. doped Si, doped Ge), metal alloys (for example based on Au, Ag, Cu, etc.), semiconductor alloys, polymers (e.g. polyvinyl chloride, polyolefins, such as polyethylene and polypropylene, polyesters, fluoropolymers, polyamides, polyimides, polyurethanes, polyethersulfones, polyalkyl (meth)acrylates, polystyrene and mixtures and composites thereof), inorganic solids (e.g. ammonium chloride), paper and combinations thereof. The substrates may be flexible or inflexible, and have a curved or planar geometry, depending on the desired use.

A typical substrate for semiconductor units comprises a matrix (for example a quartz or polymer matrix) and, optionally, a dielectric top layer.

Suitable dielectrics are SiO₂, polystyrene, poly-α-methylstyrene, polyolefins (such as polypropylene, polyethylene, polyisobutene), polyvinylcarbazole, fluorinated polymers (e.g. Cytop), cyanopullulans (e.g. CYMM), polyvinylphenol, poly-p-xylene, polyvinyl chloride, or polymers crosslinkable thermally or by atmospheric moisture. Specific dielectrics are “self-assembled nanodielectrics”, i.e. polymers which are obtained from monomers comprising SiCl functionalities, for example Cl₃SiOSiCl₃, Cl₃Si—(CH₂)₆—SiCl₃, Cl₃Si—(CH₂)₁₂—SiCl₃, and/or which are crosslinked by atmospheric moisture or by addition of water diluted with solvents (see, for example, Facchetti, Adv. Mater. 2005, 17, 1705-1725). Instead of water, it is also possible for hydroxyl-containing polymers such as polyvinylphenol or polyvinyl alcohol or copolymers of vinylphenol and styrene to serve as crosslinking components. It is also possible for at least one further polymer to be present during the crosslinking operation, for example polystyrene, which is then also crosslinked (see Facchetti, US patent application 2006/0202195).

The substrate may additionally have electrodes, such as gate, drain and source electrodes of OFETs, which are normally localized on the substrate (for example deposited onto or embedded into a nonconductive layer on the dielectric). The substrate may additionally comprise conductive gate electrodes of the OFETs, which are typically arranged below the dielectric top layer (i.e. the gate dielectric).

In a specific embodiment, an insulator layer (gate insulating layer) is present on at least part of the substrate surface. The insulator layer comprises at least one insulator which is preferably selected from inorganic insulators, such as SiO₂, silicon nitride (Si₃N₄), etc., ferroelectric insulators, such as Al₂O₃, Ta₂O₅, La₂O₅, TiO₂, Y₂O₃, etc., organic insulators such as polyimides, benzocyclobutene (BCB), polyvinyl alcohols, polyacrylates, etc., and combinations thereof.

Suitable materials for source and drain electrodes are in principle electrically conductive materials. These include metals, preferably metals of groups 6, 7, 8, 9, 10 or 11 of the Periodic Table, such as Pd, Au, Ag, Cu, Al, Ni, Cr, etc. Also suitable are conductive polymers, such as PEDOT (=poly(3,4-ethylenedioxythiophene)):PSS (=poly(styrenesulfonate)), polyaniline, surface-modified gold, etc. Preferred electrically conductive materials have a specific resistance of less than 10⁻³ ohm×meter, preferably less than 10⁻⁴ ohm×meter, especially less than 10⁻⁶ or 10⁻⁷ ohm×meter.

In a specific embodiment, drain and source electrodes are present at least partly on the organic semiconductor material. It will be appreciated that the substrate may comprise further components as used customarily in semiconductor materials or ICs, such as insulators, resistors, capacitors, conductor tracks, etc.

The electrodes may be applied by customary processes, such as evaporation or sputtering, lithographic processes or another structuring process, such as printing techniques.

The semiconductor materials may also be processed with suitable auxiliaries (polymers, surfactants) in disperse phase by printing.

In a first preferred embodiment, the deposition of at least one compound of the general formula (I) (and if appropriate further semiconductor materials) is carried out by a gas phase deposition process (physical vapor deposition, PVD). PVD processes are performed under high-vacuum conditions and comprise the following steps: evaporation, transport, deposition. It has been found that, surprisingly, the compounds of the general formula (I) are suitable particularly advantageously for use in a PVD process, since they essentially do not decompose and/or form undesired by-products. The material deposited is obtained in high purity. In a specific embodiment, the deposited material is obtained in the form of crystals or comprises a high crystalline content. In general, for the PVD, at least one compound of the general formula (I) is heated to a temperature above its evaporation temperature and deposited on a substrate by cooling below the crystallization temperature. The temperature of the substrate in the deposition is preferably within a range from about 20 to 250° C., more preferably from 50 to 200° C. It has been found that, surprisingly, elevated substrate temperatures in the deposition of the compounds of the formula (I) can have advantageous effects on the properties of the semiconductor elements achieved.

The resulting semiconductor layers generally have a thickness which is sufficient for forming a semiconductor channel which is in contact with the source/drain electrodes. The deposition can be effected under an inert atmosphere, for example, under nitrogen, argon or helium.

The deposition is effected typically at ambient pressure or under reduced pressure. A suitable pressure range is from about 10⁻⁷ to 1.5 bar.

The compound of the formula (I) is preferably deposited on the substrate in a thickness of from 10 to 1000 nm, more preferably from 15 to 250 nm. In a specific embodiment, the compound of the formula (I) is deposited at least partly in crystalline form. For this purpose, especially the above-described PVD process is suitable. Moreover, it is possible to use previously prepared organic semiconductor crystals. Suitable processes for obtaining such crystals are described by R. A. Laudise et al. in “Physical Vapor Growth of Organic Semi-Conductors”, Journal of Crystal Growth 187 (1998), pages 449-454, and in “Physical Vapor Growth of Centimeter-sized Crystals of α-Hexathiophene”, Journal of Crystal Growth 1982 (1997), pages 416-427, which are incorporated here by reference.

In a second preferred embodiment, the deposition of at least one compound of the general formula (I) (and if appropriate further semiconductor materials) is effected by spin-coating. Surprisingly, it is thus also possible to use the compounds of the formula (I) used in accordance with the invention in a wet processing method to produce semiconductor substrates. The compounds of the formula (I) should thus also be suitable for producing semiconductor elements, especially OFETs or based on OFETs, by a printing process. It is possible for this purpose to use customary printing or coating processes (inkjet, flexographic, offset, gravure; intaglio printing, nanoprinting, slot die). Preferred solvents for the use of compounds of the formula (I) in a printing process are aromatic solvents, such as toluene, xylene, etc. It is also possible to add thickening substances, such as polymers, for example polystyrene, etc., to these “semiconductor inks”. In this case, the dielectrics used are the aforementioned compounds.

In a preferred embodiment, the inventive field-effect transistor is a thin-film transistor (TFT). In a customary construction, a thin-film transistor has a gate electrode disposed on the substrate or buffer layer (the buffer layer being part of the substrate), a gate insulation layer disposed thereon and on the substrate, a semiconductor layer disposed on the gate insulator layer, an ohmic contact layer on the semiconductor layer, and a source electrode and a drain electrode on the ohmic contact layer.

In a preferred embodiment, the surface of the substrate, before the deposition of at least one compound of the general formula (I) (and if appropriate of at least one further semiconductor material), is subjected to a modification. This modification serves to form regions which bind the semiconductor materials and/or regions on which no semiconductor materials can be deposited. The surface of the substrate is preferably modified with at least one compound (C1) which is suitable for binding to the surface of the substrate and to the compounds of the formula (I). In a suitable embodiment, a portion of the surface or the complete surface of the substrate is coated with at least one compound (C1) in order to enable improved deposition of at least one compound of the general formula (I) (and if appropriate further semiconductive compounds). A further embodiment comprises the deposition of a pattern of compounds of the general formula (C1) on the substrate by a corresponding production process. These include the mask processes known for this purpose and so-called “patterning” processes, as described, for example, in U.S. Ser. No. 11/353,934, which is incorporated here fully by reference.

Suitable compounds of the formula (C1) are capable of a binding interaction both with the substrate and with at least one semiconductor compound of the general formula (I). The term “binding interaction” comprises the formation of a chemical bond (covalent bond), ionic bond, coordinative interaction, van der Waals interactions, e.g. dipole-dipole interactions etc.), and combinations thereof. Suitable compounds of the general formula (C1) are:
silane, phosphonic acids, carboxylic acids, hydroxamic acids, such as alkyltrichlorosilanes, e.g. n-octadecyltrichlorosilane; compounds with trialkoxysilane groups, e.g. alkyltrialkoxysilanes such as n-octadecyltrimethoxysilane, n-octadecyltriethoxysilane, n-octadecyltri(n-propyl)oxysilane, n-octadecyltri(isopropyl)oxysilane; trialkoxyaminoalkylsilanes, such as triethoxyaminopropylsilane and N[(3-triethoxysilyl)propyl]ethylenediamine; trialkoxyalkyl 3-glycidyl ether silanes, such as triethoxypropyl 3-glycidyl ether silane; trialkoxyallylsilanes, such as allyltrimethoxysilane; trialkoxy(isocyanatoalkyl)silanes; trialkoxysilyl(meth)acryloyloxyalkanes and trialkoxysilyl(meth)acrylamidoalkanes, such as 1-triethoxysilyl-3-acryl-oyl-oxypropane.
amines, phosphines and sulfur-comprising compounds, especially thiols.

The compound (C1) is preferably selected from alkyltrialkoxysilanes, especially n-octadecyltrimethoxysilane, n-octadecyltriethoxysilane; hexaalkyldisilazanes, and especially hexamethyldisilazane (HMDS); C₈-C₃₀-alkylthiols, especially hexadecanethiol; mercaptocarboxylic acids and mercaptosulfonic acids, especially mercaptoacetic acid, 3-mercaptopropionic acid, mercaptosuccinic acid, 3-mercapto-1-propanesulfonic acid and the alkali metal and ammonium salts thereof.

Various semiconductor architectures comprising the inventive semiconductors are also conceivable, for example top contact, top gate, bottom contact, bottom gate, or else a vertical construction, for example a VOFET (vertical organic field-effect transistor), as described, for example, in US 2004/0046182.

Preferred semiconductor architectures are the following:
1. substrate, dielectric, organic semiconductor, preferably gate, dielectric, organic semiconductor, source and drain, known as “Bottom Gate Top Contact”;
2. substrate, dielectric, organic semiconductor, preferably substrate, gate, dielectric, source and drain, organic semiconductor, known as “Bottom Gate Bottom Contact”;
3. substrate, organic semiconductor, dielectric, preferably substrate, source and drain, organic semiconductor, dielectric, gate, known as “Top Gate Bottom Contact”;
4. substrate, organic semiconductor, dielectric, preferably substrate, organic semiconductor, source and drain, dielectric, gate, known as “Top Gate Top Contact”;

The layer thicknesses are, for example, from 10 nm to 5 μm in semiconductors, from 50 nm to 10 μm in the dielectric; the electrodes may, for example, be from 20 nm to 10 μm. The OFETs may also be combined to form other components, such as ring oscillators or inverters.

A further aspect of the invention is the provision of electronic components which comprise a plurality of semiconductor components, which may be n-type and/or p-type semiconductors. Examples of such components are field-effect transistors (FETs), bipolar junction transistors (BJTs), tunnel diodes, converters, light-emitting components, biological and chemical detectors or sensors, temperature-dependent detectors, photodetectors, such as polarization-sensitive photodetectors, gates, AND, NAND, NOT, OR, TOR and NOR gates, registers, switches, timer units, static or dynamic stores and other dynamic or sequential, logical or other digital components including programmable switches.

A specific semiconductor element is an inverter. In digital logic, the inverter is a gate which inverts an input signal. The inverter is also referred to as a NOT gate. Real inverter switches have an output current which constitutes the opposite of the input current. Typical values are, for example, (0, +5V) for TTL switches. The performance of a digital inverter reproduces the voltage transfer curve (VTC), i.e. the plot of input current against output current. Ideally, it is a staged function and, the closer the real measured curve approximates to such a stage, the better the inverter is. In a specific embodiment of the invention, the compounds of the formula (I) are used as organic semiconductors in an inverter.

The compounds of the formula (I) are also particularly advantageously suitable for use in organic photovoltaics (OPVs). Preference is given to their use in solar cells which are characterized by diffusion of excited states (exciton diffusion). In this case, one or both of the semiconductor materials utilized is notable for a diffusion of excited states (exciton mobility). Also suitable is the combination of at least one semiconductor material which is characterized by diffusion of excited states with polymers which permit conduction of the excited states along the polymer chain. In the context of the invention, such solar cells are referred to as excitonic solar cells. The direct conversion of solar energy to electrical energy in solar cells is based on the internal photo effect of a semiconductor material, i.e. the generation of electron-hole pairs by absorption of photons and the separation of the negative and positive charge carriers at a p-n transition or a Schottky contact. An exciton can form, for example, when a photon penetrates into a semiconductor and excites an electron to transfer from the valence band into the conduction band. In order to generate current, the excited state generated by the absorbed photons must, however, reach a p-n transition in order to generate a hole and an electron which then flow to the anode and cathode. The photovoltage thus generated can bring about a photocurrent in an external circuit, through which the solar cell delivers its power. The semiconductor can absorb only those photons which have an energy which is greater than its band gap. The size of the semiconductor band gap thus determines the proportion of sunlight which can be converted to electrical energy. Solar cells consist normally of two absorbing materials with different band gaps in order to very effectively utilize the solar energy. Most organic semiconductors have exciton diffusion lengths of up to 10 nm. There is still a need here for organic semiconductors through which the excited state can be passed on over very large distances. It has now been found that, surprisingly, the compounds of the general formula (I) described above are particularly advantageously suitable for use in excitonic solar cells.

Organic solar cells generally have a layer structure and generally comprise at least the following layers: anode, photoactive layer and cathode. These layers are generally applied to a substrate suitable for this purpose. The structure of organic solar cells is described, for example, in US 2005/0098726 and US 2005/0224905.

The invention provides an organic solar cell which comprises a substrate with at least one cathode and at least one anode, and at least one compound of the general formula (I) as defined above as a photoactive material. The inventive organic solar cell comprises at least one photoactive region. A photoactive region may comprise two layers, each of which has a homogeneous composition and forms a flat donor-acceptor heterojunction. A photoactive region may also comprise a mixed layer and form a donor-acceptor heterojunction in the form of a donor-acceptor bulk heterojunction. Organic solar cells with photoactive donor-acceptor transitions in the form of a bulk heterojunction are a preferred embodiment of the invention.

Suitable substrates for organic solar cells are, for example, oxidic materials, polymers and combinations thereof. Preferred oxidic materials are selected from glass, ceramic, SiO₂, quartz, etc. Preferred polymers are selected from polyethylene terephthalates, polyolefins (such as polyethylene and polypropylene), polyesters, fluoropolymers, polyamides, polyurethanes, polyalkyl (meth)acrylates, polystyrenes, polyvinyl chlorides and mixtures and composites.

Suitable electrodes (cathode, anode) are in principle metals, semiconductors, metal alloys, semiconductor alloys, nanowire thereof and combinations thereof. Preferred metals are those of groups 2, 8, 9, 10, 11 or 13 of the periodic table, e.g. Pt, Au, Ag, Cu, Al, In, Mg or Ca. Preferred semiconductors are, for example, doped Si, doped Ge, indium tin oxide (ITO), fluorinated tin oxide (FTO), gallium indium tin oxide (GITO), zinc indium tin oxide (ZITO), poly(3,4-ethylenedioxythiophene) poly(styrenesulfonate) (PEDOT-PSS), etc. Preferred metal alloys are, for example, alloys based on Pt, Au, Ag, Cu, etc. A specific embodiment is Mg/Ag alloys.

The material used for the electrode facing the light (the anode in a normal structure, the cathode in an inverse structure) is preferably a material at least partly transparent to the incident light. This preferably includes electrodes which have glass and/or a transparent polymer as a carrier material. Transparent polymers suitable as carriers are those mentioned above, such as polyethylene terephthalate. The electrical contact connection is generally effected by means of metal layers and/or transparent conductive oxides (TCOs). These preferably include ITO, doped ITO, FTO (fluorine doped tin oxide), AZO (aluminum doped tin oxide), ZnO, TiO₂, Ag, Au, Pt. Particular preference is given to ITO for contact connection. For electrical contact connection, it is also possible to use a conductive polymer, for example a poly-3,4-alkylenedioxy-thiophene, e.g. poly-3,4-ethyleneoxythiophene poly(styrenesulfonate) (PEDOT).

The electrode facing the light is configured such that it is sufficiently thin to bring about only minimal light absorption but thick enough to enable good charge transport of the extracted charge carriers. The thickness of the electrode layer (without carrier material) is preferably within a range from 20 to 200 nm.

In a specific embodiment, the material used for the electrode facing away from the light (the cathode in a normal structure, the anode in an inverse structure) is a material which at least partly reflects the incident light. This includes metal films, preferably of Ag, Au, Al, Ca, Mg, In, and mixtures thereof. Preferred mixtures are Mg/Al. The thickness of the electrode layer is preferably within a range from 20 to 300 nm.

The photoactive region comprises or consists of at least one layer which comprises at least one compound of the general formula (I) as defined above. In addition, the photoactive region may have one or more further layer(s). These are, for example, selected from
layers with electron-conducting properties (electron transport layer, ETL),
layers which comprise a hole-conducting material (hole transport layer, HTL), which need not absorb any radiation,
exciton- and hole-blocking layers (e.g. EBLs), which must not absorb, and
multiplication layers.

Suitable materials for these layers are described in detail hereinafter.

Suitable exciton- and hole-blocking layers are described, for example, in U.S. Pat. No. 6,451,415.

Suitable materials for exciton-blocking layers are, for example, bathocuproin (BCP), 4,4′,4″-tris[3-methylphenyl-N-phenylamino]triphenylamine (m-MTDATA).

The inventive solar cells comprise at least one photoactive donor-acceptor heterojunction. Optical excitation of an organic material generates excitons. In order that a photocurrent occurs, the electron-hole pair has to be separated, typically at a donor-acceptor interface between two unlike contact materials. At such an interface, the donor material forms a heterojunction with an acceptor material. When the charges are not separated, they can recombine in a process also known as “quenching”, either radiatively by the emission of light of a lower energy than the incident light or nonradiatively by generation of heat. Both processes are undesired. According to the invention, at least one compound of the general formula (I) can be used as a charge generator (donor) or as electron acceptor material.

If at least one compound of the general formula (I) is used as a charge generator (donor) it can be combined with an appropriate electron acceptor material (ETM, electron transport material). Radiative excitation is followed by a rapid electron transfer to the ETM. Suitable ETMs are, for example, C60 and other fullerenes, perylene-3,4;9,10-bis(dicarboximides) (PTCDIs), or n-doped layers thereof (as described hereinafter). Preferred ETMs are C60 and other fullerenes or n-doped layers thereof.

In a first embodiment, the heterojunction has a flat configuration (see: Two layer organic photovoltaic cell, C. W. Tang, Appl. Phys. Lett., 48 (2), 183-185 (1986) or N. Karl, A. Bauer, J. Holzäpfel, J. Marktanner, M. Möbus, F. Stölzle, Mol. Cryst. Liq. Cryst., 252, 243-258 (1994).).

In a second preferred embodiment, the heterojunction is configured as a bulk (mixed) heterojunction, also referred to as an interpenetrating donor-acceptor network. Organic photovoltaic cells with a bulk heterojunction are described, for example, by C. J. Brabec, N. S. Sariciftci, J. C. Hummelen in Adv. Funct. Mater., 11 (1), 15 (2001) or by J. Xue, B. P. Rand, S. Uchida and S. R. Forrest in J. Appl. Phys. 98, 124903 (2005). Bulk heterojunctions are discussed in detail hereinafter.

The compounds of the formula (I) can be used as a photoactive material in cells with MiM, pin, pn, Mip or Min structure (M=metal, p=p-doped organic or inorganic semiconductor, n=n-doped organic or inorganic semiconductor, i=intrinsically conductive system of organic layers; see, for example, J. Drechsel et al., Org. Electron., 5 (4), 175 (2004) or Maennig et al., Appl. Phys. A 79, 1-14 (2004)).

The compounds of the formula (I) can also be used as a photoactive material in tandem cells. Suitable tandem cells are described, for example, by P. Peumans, A. Yakimov, S. R. Forrest in J. Appl. Phys., 93 (7), 3693-3723 (2003) (see also U.S. Pat. No. 4,461,922, U.S. Pat. No. 6,198,091 and U.S. Pat. No. 6,198,092) and are described in detail hereinafter. The use of compounds of the general formula (I) in tandem cells is a preferred embodiment of the invention.

The compounds of the formula (I) can also be used as a photoactive material in tandem cells which are constructed from two or more than two stacked MiM, pin, Mip or Min structures (see DE 103 13 232.5 and J. Drechsel et al., Thin Solid Films, 451452, 515-517 (2004)).

The layer thickness of the M, n, i and p layers is typically within a range from 10 to 1000 nm, more preferably from 10 to 400 nm. The layers which form the solar cell can be produced by customary processes known to those skilled in the art. These include vapor deposition under reduced pressure or in an inert gas atmosphere, laser ablation or solution or dispersion processing methods such as spincoating, knifecoating, casting methods, spray application, dipcoating or printing (e.g. inkjet, flexographic, offset, gravure; intaglio, nanoimprinting). In a specific embodiment, the entire solar cell is produced by a gas phase deposition process.

In order to improve the efficiency of organic solar cells, it is possible to shorten the mean distance through which the exciton has to diffuse in order to arrive at the next donor-acceptor interface. To this end, it is possible to use mixed layers of donor material and acceptor material which form an interpenetrating network in which internal donor-acceptor heterojunctions are possible. This bulk heterojunction is a specific form of the mixed layer, in which the excitons generated need only travel a very short distance before they arrive at a domain boundary, where they are separated.

In a preferred embodiment, the photoactive donor-acceptor transitions in the form of a bulk heterojunction are produced by a gas phase deposition process (physical vapor deposition, PVD). Suitable processes are described, for example, in US 2005/0227406, to which reference is made here. To this end, a compound of the general formula (I) and a complementary semiconductor material can be subjected to a gas phase deposition in the manner of a cosublimation. PVD processes are performed under high-vacuum conditions and comprise the following steps: evaporation, transport, deposition. The deposition is effected preferably at a pressure within a range from about 10⁻² mbar to 10⁻⁷ mbar, for example from 10⁻⁵ to 10⁻⁷ mbar. The deposition rate is preferably within a range from 0.01 to 100 nm/s. The deposition can be effected in an inert gas atmosphere, for example under nitrogen, helium or argon. The temperature of the substrate during the deposition is preferably within a range from −100 to 300° C., more preferably from −50 to 250° C.

The other layers of the organic solar cell can be produced by known processes. These include vapor deposition under reduced pressure or in an inert gas atmosphere, laser ablation, or solution or dispersion processing methods such as spincoating, knifecoating, casting methods, spray application, dipcoating or printing (e.g. inkjet, flexographic, offset, gravure; intaglio, nanoimprinting). In a specific embodiment, the entire solar cell is produced by a gas phase deposition process.

The photoactive layer (homogeneous layer or mixed layer) can be subjected to a thermal treatment directly after production thereof or after production of further layers which form the solar cell. Such a heat treatment can in many cases further improve the morphology of the photoactive layer. The temperature is preferably within a range from about 60° C. to 300° C. The treatment time is preferably within a range from 1 minute to 3 hours. In addition or alternatively to a thermal treatment, the photoactive layer (mixed layer) can be subjected to a treatment with a solvent-containing gas directly after production thereof or after production of further layers which form the solar cell. In a suitable embodiment, saturated solvent vapors in air are used at ambient temperature. Suitable solvents are toluene, xylene, chloroform, N-methylpyrrolidone, dimethylformamide, ethyl acetate, chlorobenzene, dichloromethane and mixtures thereof. The treatment time is preferably within a range from 1 minute to 3 hours.

In a suitable embodiment, the inventive solar cells are present as an individual cell with flat heterojunction and normal structure. In a specific embodiment, the cell has the following structure:
an at least partly transparent conductive layer (top electrode, anode) (11)
a hole-conducting layer (hole transport layer, HTL) (12)
a layer which comprises a donor material (13)
a layer which comprises an acceptor material (14)
an exciton-blocking and/or electron-conducting layer (15)
a second conductive layer (back electrode, cathode) (16)

The donor material preferably comprises at least one compound of the formula (I) or consists of a compound of the formula (I). The acceptor material preferably comprises at least one fullerene or fullerene derivative, or consists of a fullerene or fullerene derivative. The acceptor material preferably comprises C60 or PCBM ([6,6]-phenyl-C61-butyric acid methyl ester).

The essentially transparent conductive layer (11) (anode) comprises a carrier, such as glass or a polymer (e.g. polyethylene terephthalate) and a conductive material, as described above. Examples include ITO, doped ITO, FTO, ZnO, AZO, etc. The anode material can be subjected to a surface treatment, for example with UV light, ozone, oxygen plasma, Br₂, etc. The layer (11) should be sufficiently thin to enable maximum light absorption, but also sufficiently thick to ensure good charge transport. The layer thickness of the transparent conductive layer (11) is preferably within a range from 20 to 200 nm.

Solar cells with normal structure optionally have a hole-conducting layer (HTL). This layer comprises at least one hole-conducting material (hole transport material, HTM). Layer (12) may be an individual layer of essentially homogeneous composition or may comprise two or more than two sublayers.

Hole-conducting materials (HTM) suitable for forming layers with hole-conducting properties (HTL) preferably comprise at least one material with high ionization energy. The ionization energy is preferably at least 5.0 eV, more preferably at least 5.5 eV. The materials may be organic or inorganic materials. Organic materials suitable for use in a layer with hole-conducting properties are preferably selected from poly(3,4-ethylene-dioxythiophene) poly(styrenesulfonate) (PEDOT-PSS), Ir-DPBIC (tris-N,N′-diphenyl-benzimidazol-2-ylideneiridium(III)), N,N′-diphenyl-N,N′-bis(3-methylphenyl)-1,1′-di-phenyl-4,4′-diamine (α-NPD), 2,2′,7,7′-tetrakis(N,N-di-p-methoxyphenylamine)-9,9′-spirobifluorene (spiro-MeOTAD), etc. and mixtures thereof. The organic materials may, if desired, be doped with a p-dopant which has a LUMO within the same range as or lower than the HOMO of the hole-conducting material. Suitable dopants are, for example, 2,3,5,6-tetrafluoro-7,7,8,8-tetracyanoquinodimethane (F₄TCNQ), WO₃, MoO₃, etc. Inorganic materials suitable for use in a layer with hole-conducting properties are preferably selected from WO₃, MoO₃, etc.

If present, the thickness of the layers with hole-conducting properties is preferably within a range from 5 to 200 nm, more preferably 10 to 100 nm.

Layer (13) comprises at least one compound of the general formula (I). The thickness of the layer should be sufficient to absorb a maximum amount of light, but thin enough to enable effective dissipation of the charge. The thickness of the layer (13) is preferably within a range from 5 nm to 1 μm, more preferably from 5 to 100 nm.

Layer (14) comprises at least one acceptor material. The acceptor material preferably comprises at least one fullerene or fullerene derivative. Alternatively or additionally suitable acceptor materials are specified hereinafter. The thickness of the layer should be sufficient to absorb a maximum amount of light, but thin enough to enable effective dissipation of the charge. The thickness of the layer (14) is preferably within a range from 5 nm to 1 μm, more preferably from 5 to 80 nm.

Solar cells with normal structure optionally comprise an exciton-blocking and/or electron-conducting layer (15) (EBL/ETL). Suitable materials for exciton-blocking layers generally have a greater band gap than the materials of layer (13) and/or (14). They are firstly capable of reflecting excitons and secondly enable good electron transport through the layer. The materials for the layer (15) may comprise organic or inorganic materials. Suitable organic materials are preferably selected from 2,9-dimethyl-4,7-diphenyl-1,10-phenanthroline (BCP), 4,7-diphenyl-1,10-phenanthroline (Bphen), 1,3-bis[2-(2,2′-bipyridin-6-yl)-1,3,4-oxadiazo-5-yl]benzene (BPY-OXD), etc. The organic materials may, if desired, be doped with an n-dopant which has a HOMO within the same range as or lower than the LUMO of the electron-conducting material. Suitable dopants are, for example, Cs₂CO₃, Pyronin B (PyB), Rhodamine B, cobaltocenes, etc. Inorganic materials suitable for use in a layer with electron-conducting properties are preferably selected from ZnO, etc. If present, the thickness of the layer (15) is preferably within a range from 5 to 500 nm, more preferably 10 to 100 nm.

Layer (16) is the cathode and preferably comprises at least one compound with low work function, more preferably a metal such as Ag, Al, Mg, Ca, etc. The thickness of the layer (16) is preferably within a range from about 10 nm to 10 μm, e.g. 10 nm to 60 nm.

In a further suitable embodiment, the inventive solar cells are present as an individual cell with a flat heterojunction and inverse structure.

In a specific embodiment, the cell has the following structure:
an at least partly transparent conductive layer (cathode) (11)
an exciton-blocking and/or electron-conducting layer (12)
a layer which comprises an acceptor material (13)
a layer which comprises a donor material (14)
a hole-conducting layer (hole transport layer, HTL) (15)
a second conductive layer (back electrode, anode) (16)

With regard to suitable and preferred materials for the layers (11) to (16), reference is made to the above remarks regarding the corresponding layers in solar cells with normal structure.

In a further preferred embodiment, the inventive solar cells are present as an individual cell with normal structure and have a bulk heterojunction. In a specific embodiment, the cell has the following structure:
an at least partly transparent conductive layer (anode) (21)
a hole-conducting layer (hole transport layer, HTL) (22)
a mixed layer which comprises a donor material and an acceptor material, which form a donor-acceptor heterojunction in the form of a bulk heterojunction (23)
an electron-conducting layer (24)
an exciton-blocking and/or electron-conducting layer (25)
a second conductive layer (back electrode, cathode) (26)

The layer (23) comprises at least one compound of the general formula (I) as a photoactive material, e.g. as a donor material. The layer (23) additionally comprises a complementary semiconductor material, e.g. at least one fullerene or fullerene derivative as an acceptor material. The layer (23) comprises especially C60 or PCBM ([6,6]-phenyl-C61-butyric acid methyl ester) as an acceptor material.

With regard to layer (21), reference is made completely to the above remarks regarding layer (11).

With regard to layer (22), reference is made completely to the above remarks regarding layer (12).

Layer (23) is a mixed layer which comprises at least one compound of the general formula (I) as a semiconductor material. In addition, layer (23) comprises at least one complementary semiconductor material. As described above, the layer (23) can be produced by coevaporation or by solution processing using customary solvents. The mixed layer comprises preferably 10 to 90% by weight, more preferably 20 to 80% by weight, of at least one compound of the general formula (I), based on the total weight of the mixed layer. The mixed layer comprises preferably 10 to 90% by weight, more preferably 20 to 80% by weight, of at least one acceptor material, based on the total weight of the mixed layer. The thickness of the layer (23) should be sufficient to absorb a maximum amount of light, but thin enough to enable effective dissipation of the charge. The thickness of the layer (23) is preferably within a range from 5 nm to 1 μm, more preferably from 5 to 200 nm, especially 5 to 80 nm.

Solar cells with a bulk heterojunction comprise an electron-conducting layer (24) (ETL). This layer comprises at least one electron transport material (ETM). Layer (24) may be a single layer of essentially homogeneous composition or may comprise two or more than two sublayers. Suitable materials for electron-conducting layers generally have a low work function or ionization energy. The ionization energy is preferably not more than 3.5 eV. Suitable organic materials are preferably selected from the aforementioned fullerenes and fullerene derivatives, 2,9-dimethyl-4,7-diphenyl-1,10-phenanthroline (BCP), 4,7-diphenyl-1,10-phenanthroline (Bphen), 1,3-bis[2-(2,2′-bipyridin-6-yl)-1,3,4-oxadiazo-5-yl]benzene (BPY-OXD), etc. The organic materials used in layer (24) may, if desired, be doped with an n-dopant which has a HOMO within the same range as or lower than the LUMO of the electron-conducting material. Suitable dopants are, for example, Cs₂CO₃, Pyronin B (PyB), Rhodamine B, cobaltocenes, etc. The thickness of the layer (23) is, if present, preferably within a range from 1 nm to 1 μm, particularly 5 to 60 nm.

With regard to layer (25), reference is made completely to the above remarks regarding layer (15).

With regard to layer (26), reference is made completely to the above remarks regarding layer (16).

Solar cells with a donor-acceptor heterojunction in the form of a bulk heterojunction can be produced by a gas phase deposition process as described above. With regard to deposition rates, substrate temperature during the deposition and thermal aftertreatment, reference is made to the above remarks.

In a further preferred embodiment, the inventive solar cells are present as an individual cell with inverse structure and have a bulk heterojunction.

In a particularly preferred embodiment, the inventive solar cell is a tandem cell.

A tandem cell consists of two or more than two (e.g. 3, 4, 5, etc.) subcells. A single subcell, some of the subcells or all subcells may have photoactive donor-acceptor heterojunctions. Each donor-acceptor heterojunction may be in the form of a flat heterojunction or in the form of a bulk heterojunction. Preferably, at least one of the donor-acceptor heterojunctions is in the form of a bulk heterojunction. According to the invention, the photoactive layer of at least one subcell comprises a compound of the general formula (I). Preferably, the photoactive layer of at least one subcell comprises a compound of the general formula (I) and at least one fullerene or fullerene derivative. More preferably, the semiconductor mixture used in the photoactive layer of at least one subcell consists of a compound of the general formula (I) and C₆₀ or [6,6]-phenyl-C61-butyric acid methyl ester.

The subcells which form the tandem cell may be connected in parallel or in series. The subcells which form the tandem cell are preferably connected in series. There is preferably an additional recombination layer in each case between the individual subcells. The individual subcells have the same polarity, i.e. generally either only cells with normal structure or only cells with inverse structure are combined with one another.

The inventive tandem cell preferably comprises a transparent conductive layer (layer 31). Suitable materials are those specified above for the individual cells. Layers (32) and (34) constitute subcells. “Subcell” refers here to a cell as defined above without cathode and anode. The subcells may, for example, either all have a compound of the general formula (I) used in accordance with the invention in the photoactive layer (preferably in combination with a fullerene or fullerene derivative, especially C60) or have other combinations of semiconductor materials, for example C60 with zinc phthalocyanine, C60 with oligothiophene (such as DCV5T). In addition, individual subcells may also be configured as dye-sensitized solar cells or polymer cells.

In all cases, preference is given to a combination of materials which exploit different regions of the spectrum of the incident light, for example of natural sunlight. For instance, the combination of a compound of the general formula (I) and fullerene or fullerene derivative used in accordance with the invention absorbs in the long-wave region of sunlight. Cells based on at least one perylene compound as described, for example, in International patent application WO2011158211, absorb primarily in the short-wave range. Thus, a tandem cell composed of a combination of these subcells should absorb radiation in the range from about 400 nm to 900 nm. Suitable combination of subcells should thus allow the spectral range utilized to be extended. For optimal performance properties, optical interference should be considered. For instance, subcells which absorb at relatively short wavelengths should be arranged closer to the metal top contact than subcells with longer-wave absorption.

With regard to layer (31), reference is made completely to the above remarks regarding layers (11) and (21).

With regard to layers (32) and (34), reference is made completely to the above remarks regarding layers (12) to (15) for flat heterojunctions and (22) to (25) for bulk heterojunctions.

Layer (33) is a recombination layer. Recombination layers enable the charge carriers from one subcell to recombine with those of an adjacent subcell. Small metal clusters are suitable, such as Ag, Au or combinations of highly n- and p-doped layers. In the case of metal clusters, the layer thickness is preferably within a range from 0.5 to 5 nm. In the case of highly n- and p-doped layers, the layer thickness is preferably within a range from 5 to 40 nm. The recombination layer generally connects the electron-conducting layer of a subcell to the hole-conducting layer of an adjacent subcell. In this way, further cells can be combined to form the tandem cell.

Layer (36) is the top electrode. The material depends on the polarity of the subcells. For subcells with normal structure, preference is given to using metals with a low work function, such as Ag, Al, Mg, Ca, etc. For subcells with inverse structure, preference is given to using metals with a high work function, such as Au or Pt, or PEDOT-PSS.

In the case of subcells connected in series, the overall voltage corresponds to the sum of the individual voltages of all subcells. The overall current, in contrast, is limited by the lowest current of one subcell. For this reason, the thickness of each subcell should be optimized such that all subcells have essentially the same current.

Examples of different kinds of donor-acceptor heterojunctions are a donor-acceptor double layer with a flat heterojunction, or the heterojunction is configured as a hybrid planar-mixed heterojunction or gradient bulk heterojunction or annealed bulk heterojunction.

The production of a hybrid planar-mixed heterojunction is described in Adv. Mater. 17, 66-70 (2005). In this structure, mixed heterojunction layers which were formed by simultaneous evaporation of acceptor and donor material are present between homogeneous donor and acceptor material.

In a specific embodiment of the present invention, the donor-acceptor-heterojunction is in the form of a gradient bulk heterojunction. In the mixed layers composed of donor and acceptor materials, the donor-acceptor ratio changes gradually. The form of the gradient may be stepwise or linear. In the case of a stepwise gradient, the layer 01 consists, for example, of 100% donor material, layer 02 has a donor/acceptor ratio>1, layer 03 has a donor/acceptor ratio=1, layer 04 has a donor/acceptor ratio<1, and layer 05 consists of 100% acceptor material. In the case of a linear gradient, layer 01 consists, for example, of 100% donor material, layer 02 has a decreasing ratio of donor/acceptor, i.e. the proportion of donor material decreases in a linear manner in the direction of layer 03, and layer 03 consists of 100% acceptor material. The different donor-acceptor ratios can be controlled by means of the deposition rate of each and every material. Such structures can promote the percolation path for charges.

In a further specific embodiment of the present invention, the donor-acceptor heterojunction is configured as an annealed bulk heterojunction; see, for example, Nature 425, 158-162, 2003. The process for producing such a solar cell comprises an annealing step before or after the metal deposition. As a result of the annealing, donor and acceptor materials can separate, which leads to more extended percolation paths.

In a further specific embodiment of the present invention, the organic solar cells are produced by organic vapor phase deposition, either with a flat or a controlled heterojunction architecture. Solar cells of this type are described in Materials, 4, 2005, 37.

The organic solar cells of the invention preferably comprise at least one photoactive region which comprises at least one compound of the formula (I), which is in contact with at least one complementary semiconductor. In addition to compounds of the formula (I), the semiconductor materials listed hereinafter are suitable in principle for use in solar cells according to the invention.

Preferred further semiconductors are fullerenes and fullerene derivatives, preferably selected from C₆₀, C₇₀, C₈₄, phenyl-C₆₁-butyric acid methyl ester ([60]PCBM), phenyl-C₇₁-butyric acid methyl ester ([71]PCBM), phenyl-C₈₄-butyric acid methyl ester ([84]PCBM), phenyl-C₆₁-butyric acid butyl ester ([60]PCBB), phenyl-C₆₁-butyric acid octyl ester ([60]PCBO), thienyl-C₆₁-butyric acid methyl ester ([60]ThCBM) and mixtures thereof. Particular preference is given to C₆₀, [60]PCBM and mixtures thereof. Preference is given to those fullerenes which are vaporizable, for example C60 or C70. Fullerenes and fullerene derivatives in combination with at least one compound of the formula (I) usually act as acceptors.

Suitable further semiconductors are perylendiimides different from the compounds of formula (I). Suitable are e.g. perylendiimides of the formula

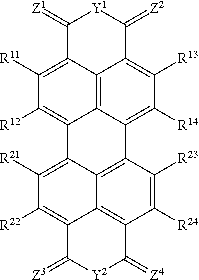

in which
the R¹¹, R¹², R¹³, R¹⁴, R²¹R²², R²³ and R²⁴ radicals are each independently hydrogen, halogen or groups other than halogen,
Y¹ is O or NR^{a} where R^{a} is hydrogen or an organyl radical,
Y² is O or NR^{b} where R^{b} is hydrogen or an organyl radical,
Z¹, Z², Z³ and Z⁴ are each O,
where, in the case that Y¹ is NR^{a}, one of the Z¹ and Z² radicals may also be NR^{c}, where the R^{a} and R^{c} radicals together are a bridging group having 2 to 5 atoms between the flanking bonds, and
where, in the case that Y² is NR^{b}, one of the Z³ and Z⁴ radicals may also be NR^{d}, where the R^{b} and R^{d} radicals together are a bridging group having 2 to 5 atoms between the flanking bonds.

Suitable perylendiimides are, for example, described in WO 2007/074137, WO 2007/093643 and WO 2007/116001, to which reference is made here.

Perylendiimides in combination with at least one compound of the formula (I) may act as donors or acceptors, depending inter alia on the substituents of the perylene diimides.

Further suitable semiconductors are thiophene compounds. These are preferably selected from thiophenes, oligothiophenes and substituted derivatives thereof. Suitable oligothiophenes are quaterthiophenes, quinquethiophenes, sexithiophenes, α,ω-di(C₁-C₈)-alkyloligothiophenes, such as α,ω-dihexylquaterthiophenes, α,ω-dihexylquinquethiophenes and α,ω-dihexylsexithiophenes, poly(alkylthiophenes) such as poly(3-hexylthiophene), bis(dithienothiophenes), anthradithiophenes and dialkylanthradithiophenes such as dihexylanthradithiophene, phenylene-thiophene (P-T) oligomers and derivatives thereof, especially α,ω-alkyl-substituted phenylene-thiophene oligomers.

Further thiophene compounds suitable as semiconductors are preferably selected from compounds like

α,α′-bis(2,2-dicyanovinyl)quinquethiophene (DCV5T),
(3-(4-octylphenyl)-2,2′-bithiophene) (PTOPT),
and acceptor-substituted oligothiophenes as described in WO 2006/092124.

Thiophene compounds in combination with at least one compound of the formula (I) usually act as donors.

Further semiconductors suitable as donors are merocyanines as described in WO 2010/049512.

All aforementioned semiconductors may be doped. The conductivity of semiconductors can be increased by chemical doping techniques using dopants. An organic semiconductor material may be doped with an n-dopant which has a HOMO energy level which is close to or higher than the LUMO energy level of the electron-conducting material. An organic semiconductor material may also be doped with a p-dopant which has a LUMO energy level which is close to or higher than the HOMO energy level of the hole-conducting material. In other words, in the case of n-doping an electron is released from the dopant, which acts as the donor, whereas in the case of p-doping the dopant acts as an acceptor which accepts an electron.

Suitable dopants for the compounds (I) according to the invention and for p-semiconductors in general are, for example, selected from WO₃, MoO₃, 2,3,5,6-tetrafluoro-7,7,8,8-tetracyanoquinodimethane (F₄-TCNQ), 3,6-difluoro-2,5,7,7,8,8-hexacyanoquinodimethane, dichlorodicyanoquinone (DDQ) or tetracyanoquinodimethane (TCNQ). A preferred dopant is 3,6-difluoro-2,5,7,7,8,8-hexacyanoquinodimethane.

Further suitable dopants are, for example, selected from Cs₂CO₃, LiF, Pyronin B (PyB), rhodamine derivatives, cobaltocenes, etc. Preferred dopants are Pyronin B and rhodamine derivatives, especially rhodamine B.

The dopants are typically used in an amount of up to 10 mol %, preferably up to 5 mol %, based on the amount of the semiconductor to be doped.

The invention further provides an electroluminescent (EL) arrangement comprising an upper electrode, a lower electrode, wherein at least one of said electrodes is transparent, an electroluminescent layer and optionally an auxiliary layer, wherein the electroluminescent arrangement comprises at least one compound of the formula I as defined above. An EL arrangement is characterized by the fact that it emits light when an electrical voltage is applied with flow of current. Such arrangements have been known for a long time in industry and technology as light-emitting diodes (LEDs). Light is emitted on account of the fact that positive charges (holes) and negative charges (electrons) combine with the emission of light. In the sense of this application the terms electroluminescing arrangement and organic light-emitting diode (OLEDs) are used synonymously. As a rule, EL arrangements are constructed from several layers. At least on of those layers contains one or more organic charge transport compounds. The layer structure is in principle as follows:

1. Carrier, substrate
2. Base electrode (anode)
3. Hole-injecting layer
4. Hole-transporting layer
5. Light-emitting layer
6. Electron-transporting layer
7. Electron-injecting layer
8. Top electrode (cathode)

### 9. Contacts

10. Covering, encapsulation.

This structure represents the most general case and can be simplified by omitting individual layers, so that one layer performs several tasks. In the simplest case an EL arrangement consists of two electrodes between which an organic layer is arranged, which fulfils all functions, including emission of light. The structure of organic light-emitting diodes and processes for their production are known in principle to those skilled in the art, for example from WO 2005/019373. Suitable materials for the individual layers of OLEDs are disclosed, for example, in WO 00/70655. Reference is made here to the disclosure of these documents. In principle OLEDs according to the invention can be produced by methods known to those skilled in the art. In a first embodiment, an OLED is produced by successive vapor deposition of the individual layers onto a suitable substrate. For vapor deposition, it is possible to use customary techniques such as thermal evaporation, chemical vapor deposition and others. In an alternative embodiment, the organic layers may be coated from solutions or dispersions in suitable solvents, for which coating techniques known to those skilled in the art are employed.

Suitable as substrate 1 are transparent carriers, such as glass or plastics films (for example polyesters, such as polyethylene terephthalate or polyethylene naphthalate, polycarbonate, polyacrylate, polysulphone, polyimide foil). Suitable as transparent and conducting materials are a) metal oxide, for example indium-tin oxide (ITO), tin oxide (NESA), etc. and b) semi-transparent metal films, for example Au, Pt, Ag, Cu, etc.

The compounds of the formula (I) preferably serve as a charge transport material (electron conductor). Thus, at least one compound of the formula I as defined above is preferably used in a hole-injecting layer, hole transporting layer or as part of a transparent electrode.

In the EL applications according to the invention low molecular weight or oligomeric as well as polymeric materials may be used as light-emitting layer 5. The substances are characterized by the fact that they are photoluminescing. Accordingly, suitable substances are for example fluorescent dyes and fluorescent products that are forming oligomers or are incorporated into polymers. Examples of such materials are coumarins, perylenes, anthracenes, phenanthrenes, stilbenes, distyryls, methines or metal complexes such as Alq₃ (tris(8-hydroxyquinolinato)aluminium), etc. Suitable polymers include optionally substituted phenylenes, phenylene vinylenes or polymers with fluorescing segments in the polymer side chain or in the polymer backbone. A detailed list is given in EP-A-532 798. Preferably, in order to increase the luminance, electron-injecting or hole-injecting layers (3 and/or 7) can be incorporated into the EL arrangements. A large number of organic compounds that transport charges (holes and/or electrons) are described in the literature. Mainly low molecular weight substances are used, which are for example vacuum evaporated in a high vacuum. A comprehensive survey of the classes of substances and their use is given for example in the following publications: EP-A 387 715, U.S. Pat. No. 4,539,507, U.S. Pat. No. 4,720,432 and U.S. Pat. No. 4,769,292. A preferred material is PEDOT (poly-(3,4-ethylenedioxythiophene)) which can also be employed in the transparent electrode of the OLEDs.

As a result of the inventive use of the compounds (I), it is possible to obtain OLEDs with high efficiency. The inventive OLEDs can be used in all devices in which electroluminescence is useful. Suitable devices are preferably selected from stationary and mobile visual display units. Stationary visual display units are, for example, visual display units of computers, televisions, visual display units in printers, kitchen appliances and advertising panels, illuminations and information panels. Mobile visual display units are, for example, visual display units in cell phones, laptops, digital cameras, vehicles and destination displays on buses and trains. Moreover, the compounds (I) may be used in OLEDs with inverse structure. The compounds (I) in these inverse OLEDs are in turn preferably used in the light-emitting layer. The structure of inverse OLEDs and the materials typically used therein are known to those skilled in the art.

Before they are used as charge transport materials or exciton transport materials, it may be advisable to subject the compounds of the formula (I) to a purification process. Suitable purification processes comprise conventional column techniques and conversion of the compounds of the formula (I) to the gas phase. This includes purification by sublimation or PVD (physical vapor deposition).

### Polymer Composition

A further object of the invention is a composition comprising at least one compound of the formula (I) as defined above and at least one polymer, preferably at least one thermoplastic polymer. With regard to suitable and preferred compounds of the formula (I) reference is made to the suitable and preferred compounds of the formula (I) as mentioned before.

It has been surprising found that the compounds of the general formula (I) have advantageous properties as colorants for use in polymer compositions.

Advantageously, the compounds of the general formula (I) are compatible with a wide range of different polymers. In particular, they are characterized by a good solubility in different classes of polymers. They have excellent processing behaviour and good fastness properties. Further, the compounds of the general formula (I) are capable of forming transparent colored polymer compositions.

In a special embodiment, the compounds of the general formula (I) are used in a polymer composition comprising at least one thermoplastic polymer.

Preferably, the thermoplastic polymer is selected from
homo- and copolymers which comprise at least one copolymerized monomer selected from C₂-C₁₀-monoolefins, 1,3-butadiene, 2-chloro-1,3-butadiene, vinyl alcohol and its C₂-C₁₀-alkyl esters, vinyl chloride, vinylidene chloride, vinylidene fluoride, tetrafluoroethylene, glycidyl acrylate, glycidyl methacrylate, acrylates and methacrylates of C₁-C₁₀-alcohols, vinylaromatics, (meth)acrylonitrile, maleic anhydride, and α,β-ethylenically unsaturated mono- and dicarboxylic acids,
homo- and copolymers of vinyl acetals,
polyvinyl esters,
polycarbonates,
polyesters,
polyethers,
polyether ketones,
thermoplastic polyurethanes,
polysulfides,
polysulfones,
polyether sulfones,
cellulose alkyl esters,
   and mixtures thereof.

Mention may be made by way of example of polyacrylates having identical or different alcohol moieties from the group of the C₄-C₈-alcohols, particularly of butanol, hexanol, octanol, and 2-ethylhexanol, polymethyl methacrylate (PMMA), methyl methacrylate-butyl acrylate copolymers, acrylonitrile-butadiene-styrene copolymers (ABSs), ethylene-propylene copolymers, ethylene-propylene-diene copolymers (EPDMs), polystyrene (PS), styrene-acrylonitrile copolymers (SANs), acrylonitrile-styrene-acrylate (ASA), styrene-butadiene-methyl methacrylate copolymers (SBMMAs), styrene-maleic anhydride copolymers, styrene-methacrylic acid copolymers (SMAs), polyoxymethylene (POM), polyvinyl alcohol (PVAL), polyvinyl acetate (PVA), polyvinyl butyral (PVB), polycaprolactone (PCL), polyhydroxybutyric acid (PHB), polyhydroxyvaleric acid (PHV), polylactic acid (PLA), ethylcellulose (EC), cellulose acetate (CA), cellulose propionate (CP), and cellulose acetate/butyrate (CAB).

The thermoplastic polymer useful for coloration according to this invention are especially polyester, polycarbonate (PC), polystyrene (PS), polymethyl methacrylate (PMMA), polyvinylchloride, polyamide, polyethylene, polypropylene, styrene/acrylonitrile (SAN) or acrylonitrile/butadiene/styrene (ABS). Particular preference is given to polyester, polycarbonate, polystyrene, polyvinylchloride and PMMA.

The compound of the formula (I) is especially used in a molding composition comprising at least one elastomer and at least one compound of the general formula (I). The elastomer comprised in the molding compositions of the invention is preferably at least one natural rubber (NR), at least one rubber produced by a synthetic route, or a mixture thereof. Examples of preferred rubbers produced by a synthetic route are polyisoprene rubber (IR), styrene-butadiene rubber (SBR), butadiene rubber (BR), nitrile-butadiene rubber (NBR), and chloroprene rubber (CR).

For the purposes of the invention, the polymer composition can comprise at least one further additive in addition to the above constituents. Suitable additives are plastizisers, stabilizers, lubricants, fillers, pigments, flame retardants, light stabilizers, blowing agents, polymeric processing aids, impact modifiers, optical brighteners, antistatic agents, biostabilizers, etc.

The polymer composition of the invention can be used in a wide variety of products. These are e.g. packaging for food or drink, products for the interior sector, toys and child-care items, sports and leisure products, apparel, fibers for textiles, medical products, hygiene products, and the like.

The packaging that can be produced from the polymer composition of the invention for food or drink are for example freshness-retention foils, food-or-drink hoses, drinking-water hoses, containers for storing or freezing food or drink, lid gaskets, closure caps, crown corks, or synthetic corks for wine.

The products which can be produced from the polymer composition of the invention for the interior sector are for example floorcoverings, which can have homogeneous structure or a structure composed of a plurality of layers, composed of at least one foamed layer, examples being sports floors and other floorcoverings, luxury vinyl tiles (LVT), synthetic leather, wallcoverings, or foamed or unfoamed wallpapers in buildings, or are cladding or console covers in vehicles.

The toys and child-care items which can be produced from the polymer composition of the invention are for example dolls, inflatable toys, such as balls, toy figures, modeling clays, swimming aids, stroller covers, baby-changing mats, bedwarmers, teething rings, or bottles.

The sports and leisure products that can be produced from the polymer composition of the invention are for example gymnastics balls, exercise mats, seat cushions, massage balls and massage rolls, shoes and shoe soles, balls, air mattresses, and drinking bottles.

The medical products which can be produced from the polymer composition of the invention are for example tubes for enteral nutrition and hemodialysis, breathing tubes, infusion tubes, infusion bags, blood bags, catheters, tracheal tubes, gloves, breathing masks, or disposal syringes.

In a further special embodiment, the compounds of the general formula (I) are employed in surface coatings. They are in particular suitable as or in the colored layer of a coating composition. The compounds of the general formula (I) are suitable for producing multicoat color systems that are used e.g. in the automotive industry for the finishing of automobiles.

A typical coating composition comprises one or more of the following components:

(A) at least one primer,
(B) at least one color and/or effect basecoat, and
(C) at least one clearcoat.

The compounds of the general formula (I) can be used advantageously in the color coat or effect basecoat of the coating composition.

Coating compositions can be coated on the article by any of a number of techniques well-known in the art. These include, for example, spray coating, dip coating, roll coating, curtain coating, and the like. For automotive body panels, spray coating is preferred.

Colored coating compositions for the formation of a single layer on a substrate or for the formation of a composite coating are well-known in the art, and do not require explanation in detail herein. Polymers known in the art to be useful in coating compositions include acrylics, vinyls, polyurethanes, polycarbonates, polyesters, alkyds, polysiloxanes, etc. Preferred polymers include acrylics and polyurethanes. In one embodiment, the coating composition may also utilize a carbamate-functional acrylic polymer. Polymers for use in coating compositions are preferably crosslinkable, and thus comprise one or more type of cross-linkable functional groups. Such groups include, for example, hydroxy, isocyanate, amine, epoxy, acrylate, vinyl, silane, and acetoacetate groups. These groups may be masked or blocked in such a way so that they are unblocked and available for the cross-linking reaction under the desired curing conditions, generally elevated temperatures and/or actinic radiation.

The substrates to be coated may be made of any of a wide variety of materials. Examples of suitable materials are wood, glass, leather, plastics, metals, especially reactive utility metals, such as iron, steel, stainless steel, zinc, aluminum, titanium and their alloys with one another and with other metals; minerals, especially fired and unfired clay, ceramic, natural stone and artificial stone; foams; fiber materials, especially glass fibers, ceramic fibers, carbon fibers, textile fibers, polymer fibers or metal fibers, and composite fibers; or fiber reinforced materials, especially plastics reinforced with the abovementioned fibers.

The compounds of the general formula (I) can be used with preference for coating motor vehicle bodies, especially commercial and passenger vehicle bodies, and also parts, especially mounted components, thereof, the inside and outside of buildings and parts thereof, doors, windows, furniture, and hollow glassware, and, in the context of industrial coatings, for coating coils, containers, packaging, small parts, such as nuts, bolts, wheel rims or hubcaps, electrical components, such as wound products (coils, stators, rotors); and components for white goods, such as radiators, domestic appliances, refrigerator casings or washing machine casings.

The compounds of the general formula (I) can be used with preference for preparing inks, for printing inks in printing processes, for flexographic printing, screen printing, packaging printing, security color printing, intaglio printing or offset printing, for print precursors and also for textile printing, for office applications, home applications or graphic applications such as, for example, for paper goods, for ballpoint pens, felt-tippens, fibre-tip pens, paperboard, wood, (wood)stains, metal, stamp pads or inks for impact printing processes (involving impact printing color ribbons), for preparing colorants, for textile decoration and for industrial marking, for roll coatings or powder coatings or for automotive coatings, for high solids (low solvent), aqueous or metallic coatings or for pigmented formulations or aqueous paints, for mineral oils, greases or waxes, for preparing colored plastics for coatings, fibres, platters or mould carriers, for preparing non-impact printing material for digital printing, for the thermal wax transfer printing process, the ink jet printing process or for the thermal transfer printing process, or for preparing polymeric color particles, toners, dry copy toners, liquid copy toners or electrophotographic toners.

The invention is illustrated in detail with reference to the following nonrestrictive examples.

### EXAMPLES

### General Information

Unless stated otherwise, all other reagents and solvents were obtained from commercial suppliers and were used without further purification. The reactions were performed using standard vacuum-line and Schlenk techniques, work-up and purification of all compounds were performed under air and with reagent-grade solvents. Column chromatography was done with silica gel (particle size 0.063-0.200 mm) from Macherey-Nagel) and silica coated aluminum sheets with fluorescence indicator from Macherey-Nagel were used for thin layer chromatography. The ¹H-NMR and ¹³C-NMR spectra were recorded on a Bruker AVANCE 300, Bruker AVANCE III 500 and Bruker AVANCE III 700 spectrometer in the listed deuterated solvents. The control of the temperature was realized with a VTU (variable temperature unit) and an accuracy of +/−0.1K, which was monitored with the standard Bruker Topspin 3.1 software. Residual non-deuterated solvent was used as an internal standard. Solution UV-Vis absorption and emission spectra were recorded at room temperature on a Perkin-Elmer Lambda 900 spectrophotometer and J&MTIDAS spectrofluorometer in CH₂Cl₂ in a conventional quartz cell (light pass 10 mm). High-resolution electrospray ionization mass spectrometry was performed on a Q-Tof Ultima 3 (micromass/Waters). High resolution MALDI-TOF spectra were recorded on a Waters Synapt G2-Si spectrometer with C60 as reference. Cyclic voltammetry measurements were performed with a WaveDriver 20 Bipotentiostat/Galvanostat (Pine Instruments Company). High-performance-liquid chromatography was performed on an Agilent 1200 series.

Abbreviations used: DCM stands for dichloromethane; (CDCl₂)₂ stands for deuterotetrachloroethane; THF stands for tetrahydrofuran.

### I. Preparation of Compounds of the General Formula I

### Example 1

### Example 1.1

### Preparation of 4-tert-butyl-2,6-diacetyl aniline

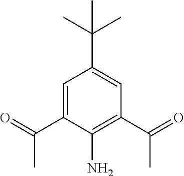 [SMILES: CC(=O)c1cc(C(C)(C)C)cc(C(C)=O)c1N]

4-tert-butyl-2,6-dibromo aniline (6 g; 19.5 mmol) and 200 mL dry dioxane were mixed and thoroughly degassed by purging with argon for 30 min. Then 20 mL tributyl(1-ethoxyvinyl)tin (59.3 mmol) were added and the system was degassed for additional 5 minutes. 0.2 eq tetrakis(triphenylphosphine)palladium (4.5 g; 3.9 mmol) were added under argon. The reaction mixture was heated to 100° C. and stirred for 20 hours. After cooling to room temperature 500 mL 2M HCl solution was added and vigorously stirred for additional 2 hours. The reaction mixture was first extracted with ethyl acetate and then with DCM. The combined organic layers were washed with brine and dried with sodium sulfate. After evaporation the residue was purified by column chromatography (silica, PE/EtOAc 95/5). The product was obtained as yellow needles after recrystallization from hot water/methanol 10/1 with 70% yield (3.2 g; 13.7 mmol).

¹H NMR (300 MHz, CD₂Cl₂, 298K) δ 1.34 (s, 9H), 2.59 (s, 6H), 8.00 (s, 2H), 9.04 (s, 2H).

¹³C NMR (75 MHz, CD₂Cl₂, 298K) δ 28.71 (2C, CH₃), 31.57 (3C, C—CH₃), 34.25 (1C, CH), 119.78, 135.73, 136.00, 151.37, 200.99 (2C, C═O).

FD mass spectrum (8 kV): m/z (%): calcd for 233.31. found: 233.90 (100) [M]⁺.

### Example 1.2

### Preparation of 2-(tert-butyl)naphtho[1′,8′:7,8,9]quinolizino[3,4,5,6-ija]quinoline-4,13-dione

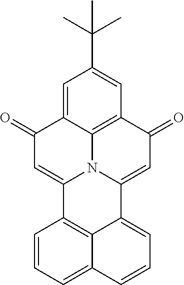 [SMILES: CC(C)(C)c1cc2c(=O)cc3c4cccc5cccc(c54)c4cc(=O)c(c1)c2n34]

In a flame dried 50 mL Schlenk flask 500 mg (2.52 mmol) of naphthaline mono anhydride, 650 mg (1.1 eq; 2.78 mmol) of 4-tert-butyl-2,6-diacetyl aniline, 0.1 eq zinc acetate (47 mg, 0.25 mmol) and 17 g imidazole were added. The solids were mixed by careful shaking and degassed three times. It was heated to 160° C. under argon for 16 h. After cooling to 100° C. water was added to the melt. The resulting solution was poured into 1M HCl solution. The precipitate was filtered and washed with water. The filter cake was eluted with DCM, then with a mixture of DCM/THF 10/1 and evaporated. The residue was purified by column chromatography (silica; DCM/THF 20/1). The product was crystallized from DCM/Hexane to give the pure compound as yellow-orange needles with in yield of 25-52% (240-480 mg; 0.64-1.27 mmol).

¹H NMR (300 MHz, CD₂Cl₂, 298K) δ 1.52 (s, 9H), 7.44 (s, 2H), 7.78 (t, J=7.94 Hz, 2H), 8.16 (d, J=8.1 Hz, 2H), 8.45 (d, J=7.68 Hz, 2H), 8.80 (s, 2H).

¹³C NMR (75 MHz, CD₂Cl₂, 298K) δ 1.33, 31.57 (3C, CH₃), 35.23 (1C, C—CH₃), 110.35, 123.64, 126.04, 126.99, 127.99, 129.06, 132.17, 143.29, 150.59, 177.83 (2C, C═O).

FD mass spectrum (8 kV): m/z (%): calcd for 377.43. found: 377.40 (100) [M]⁺.

UV-Vis (CH₂Cl₂): λₘₐₓ=416 nm (26800 m⁻¹cm⁻¹).

The structure has been proven by XRD.

### Example 1.3

### Preparation of 2,2′-(2-(tert-butyl)naphtho[1′,8′:7,8,9]quinolizino[3,4,5,6-ija]quinoline-4,13-diylidene)dimalononitrile

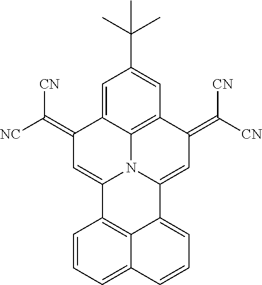 [SMILES: CC(C)(C)c1cc2c3c(c1)c(=C(C#N)C#N)cc1c4cccc5cccc(c(cc2=C(C#N)C#N)N31)c54]

2-(tert-butyl)naphtho[1′,8′:7,8,9]quinolizino[3,4,5,6-ija]quinoline-4,13-dione (70 mg; 0.185 mmol) was dissolved in a mixture of 3.5 mL glacial acetic acid and 7 mL acetic anhydride. Malononitrile (123 mg; 1.85 mmol) was added and the reaction mixture was refluxed under argon for 24 hours. Another portion of malonitrile was added and refluxing continued for 24 hours. After a total reaction time of 48 hours no starting material can be observed and the solvent was evaporated under reduced pressure. The residue was purified by column chromatography (silica; DCM) to yield 36 mg (76 μmol; 41%) product as purple shiny powder.

¹H NMR (300 MHz, CD₂Cl₂, 298K) δ 1.38 (s, 9H), 7.85 (t, J=8.31 Hz, 2H), 8.06 (s, 2H), 8.24 (d, J=8.04 Hz, 2H), 8.53 (d, J=8.19 Hz, 2H), 9.49 (s, 2H).

FD mass spectrum (8 kV): m/z (%): calcd for 473.53. found: 472.50 (100) [M]⁺.

UV-Vis (CH₂Cl₂): λₘₐₓ=630 nm (23300 m⁻¹cm⁻¹), 582 nm (21600 m⁻¹cm⁻¹), 514 nm (19800 m⁻¹cm⁻¹), 380 nm (14000 m⁻¹cm⁻¹).

The structure has been proven by XRD.

### Example 2

### Example 2.1

### Preparation of N-(2,6-dibromo-4-(tert-butyl)phenyl)phthalamide

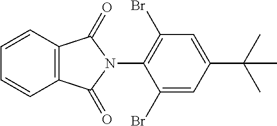 [SMILES: CC(C)(C)c1cc(Br)c(N2C(=O)c3ccccc3C2=O)c(Br)c1]

A mixture of phthalic anhydride (5.00 g, 33.76 mmol) and 2,6-dibromo-4-tert-butyl aniline (12.44 g, 40.51 mmol) was dissolved in 50 mL of acetic acid and refluxed for 12 h. The reaction was monitored by TLC (petroleum ether/ethyl acetate, 5/1), upon completion the solvent was evaporated under reduced pressure and the residue was recrystallized from methanol to give the desired product as colorless well-defined crystalls. Yield 11.60 g (78%).

¹H NMR (300 MHz, CDCl₃, 298K) δ 1.34 (s, 9H), 7.67 (s, 2H), 7.83 (dd, J=5.5, 3.1 Hz, 2H), 8.00 (dd, J=5.5, 3.1 Hz, 2H).

¹³C NMR (75 MHz, CDCl₃, 298K) δ 31.14 (3C, CH₃), 35.35 (1C, C—CH₃), 124.26, 124.64, 129.99, 131.97, 134.69, 156.20, 165.97 (2C, C═O).

FD mass spectrum (8 kV): m/z (%): calculated: 437.13. found: 437.20 (100) [M]⁺.

### Example 2.2

### Preparation of N-(2,6-diacetyl-4-(tert-butyl)phenyl)phthalimide

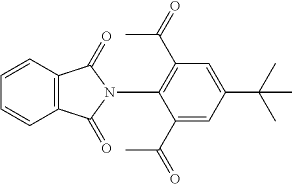 [SMILES: CC(=O)c1cc(C(C)(C)C)cc(C(C)=O)c1N1C(=O)c2ccccc2C1=O]

A solution of N-(2,6-dibromo-4-(tert-butyl)phenyl)phthalimide (2.19 g; 5.0 mmol), Pd(PPh₃)₄ (347 mg; 0.03 mmol) in 1,4-dioxane (80 mL) was stirred for 10 min under a positive flow of argon. To the yellow solution tributyl(1-ethoxy-1-ethenyl)stannane (4.23 mL; 12.5 mmol) dissolved in 1,4-dioxane (10 mL) was added. The yellow solution was heated at reflux (24 h) whereupon a dark brown solution was formed. The progress of the reaction was monitored by TLC (hexanes) to completion. The reaction mixture was cooled to ambient temperature, 50 mL 1N HCl were added and stirred for 5 h at room temperature. The reaction mixture was extracted with DCM and dried under MgSO₄. Filtration and removal of solvent gave brown viscous oil which was purified by chromatography (hexanes/EtOAc, 2/1) to give the product as colorless crystals. Yield 1.31 g (72%).

¹H NMR (300 MHz, CDCl₃, 298K) δ 1.42 (s, 9H), 2.55 (s, 6H), 7.76 (dd, J=5.5, 3.1 Hz, 2H)., 7.88-7.92 (m, 4H).

¹³C NMR (75 MHz, CDCl₃, 298K) δ 29.09 (2C, CH₃), 31.26 (3C, CH₃), 35.21 (1C, C—CH₃), 124.02, 128.85, 132.37, 134.44, 138.28, 152.67, 167.84 (2C, C═O), 199.36 (2C, C═O).

FD mass spectrum (8 kV): m/z (%): calculated: 363.41. found: 362.90 (100) [M]⁺.

UV-Vis (CH₂Cl₂): λₘₐₓ=292 nm (3600 M⁻¹cm⁻¹).

### Example 2.3

### Preparation of 2-(tert-butyl)benzo[1,2]indolizino[6,5,4,3-ija]quinoline-4,11-dione

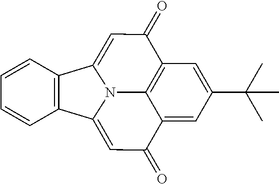 [SMILES: CC(C)(C)c1cc2c(=O)cc3c4ccccc4c4cc(=O)c(c1)c2n34]

### Route (a)

To a suspension of N-(2,6-diacetyl-4-(tert-butyl)phenyl)phthalimide (100 mg; 0.28 mmol) in acetonitrile (3 mL) 1,5-diazabicyclo[4.3.0]non-5-ene (DBN) (0.1 ml; 0.83 mmol) was added drop-wise, thus forming a clear yellow solution. The yellow crude, which precipitates after 1-2 h of stirring at room temperature was filtered and washed with a small amount of acetonitrile. The crude product was purified by a short column chromatography (CH₂Cl₂) or by recrystallization from a CH₂Cl₂/hexane mixture to give the product as yellow needles. Yield 62 mg (68%).

### Route (b)

A mixture of N-(2,6-diacetyl-4-(tert-butyl)phenyl)phthalimide (200 mg) and imidazole (2-3 g) was stirred at 120° C. for 2 h. The reaction mixture was cooled to ambient temperature, and then 20 mL of water were added and the mixture was stirred for 30 min at room temperature. The yellow suspension was filtrated and washed several times with small portions of water and methanol. The crude product could be purified by the short column chromatography (CH₂Cl₂) or by recrystallization from CH₂Cl₂-hexane mixtures to give the title compound as yellow needles. Yield: 124 mg (68%).

¹H NMR (300 MHz, CD₂Cl₂, 298K) δ 1.45 (s, 9H), 6.71 (s, 2H), 7.64 (dd, J=5.7, 3.1 Hz, 2H), 7.87 (dd, J=5.6, 3.1 Hz, 2H), 8.52 (s, 2H).

¹³C NMR (75 MHz, CD₂Cl₂, 298K) δ 31.44 (3C, CH₃), 35.66 (1C, C—CH₃), 107.21 (2C, CH), 123.63, 124.46, 129.67, 132.49, 133.54, 133.92, 145.92, 149.78, 179.75 (2C, C═O).

FD mass spectrum (8 kV): m/z (%): calcd for 327.38. found: 328.40 (100) [M]⁺.

UV-Vis (CH₂Cl₂): λₘₐₓ=449, 422 nm (29300, 15250 M⁻¹cm⁻¹).

### Example 2.4

### Preparation of 2,2′-(2-(tert-butyl)benzo[1,2]indolizino[6,5,4,3-ija]quinoline-4,11-diylidene)dimalononitrile

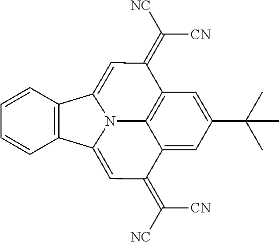 [SMILES: CC(C)(C)c1cc2c(=C(C#N)C#N)cc3c4ccccc4c4cc(=C(C#N)C#N)c(c1)c2n34]

### Route (a)

Malononitrile (60.5 mg; 0.9 mmol), TiCl₄ (0.10 mL; 0.9 mmol), and pyridine (0.22 mL; 2.8 mmol) were added to 2-(tert-butyl)benzo[1,2]indolizino[6,5,4,3-ija]quinoline-4,11-dione (100 mg; 0.3 mmol) in CH₂Cl₂ (10 mL), and the mixture was stirred at room temperature for 12 h. Every 2 h, the reaction was monitored by TLC (CH₂Cl₂) and identical amounts of malononitrile, TiCl₄, and pyridine were added until completion. The mixture was poured on ice/water and extracted with CH₂Cl₂ (3×30 mL). The combined organic layers were dried (MgSO₄) and concentrated in vacuo. The crude product was purified by short column chromatography (CH₂Cl₂) to yield 91 mg (70%) of the product as flat crystals with a gold color and metallic sheen.

### Route (b)

2-(tert-Butyl)benzo[1,2]indolizino[6,5,4,3-ija]quinoline-4,11-dione (100 mg) and malononitrile (300 mg, 4.54 mmol) were added to a mixture of acetic acid and acetic anhydride (2:1) and the mixture was stirred at 120° C. The reaction was monitored by TLC (CH₂Cl₂) and additional amounts of malononitrile were added until the completion. Then solvents were evaporated until dryness. The crude product was purified by the flesh column chromatography as flat crystals with a gold color and metallic sheen.

Yield: 91 mg (70%).

¹H NMR (300 MHz, CD₂Cl₂, 298K) δ 1.48 (s, 9H), 7.58 (s, 2H), 7.75 (dd, J=5.8, 3.0 Hz, 2H), 8.05 (dd, J=5.7, 3.1 Hz, 2H), 9.45 (s, 2H).

¹³C NMR (75 MHz, C₂D₂Cl₄, 298K) δ 31.13 (3C, CH₃), 36.43 (1C, C—CH₃), 53.84 (2C, C—CN), 106.12 (2C, CH), 119.63, 120.34, 123.63, 130.16, 131.31, 133.20, 140.38, 151.17, 152.05.

FD mass spectrum (8 kV): m/z (%): calcd for 423.47. found: 423.20 (100) [M]⁺.

UV-Vis (CH₂Cl₂): λₘₐₓ=629, 580 nm (79700, 39 420 M⁻¹cm⁻¹).

### Example 3

### Example 3.1

### Preparation of N,N′-bis(2,6-dibromophenyl)-perylene-3,4,9,10-tetracarboxybisimide

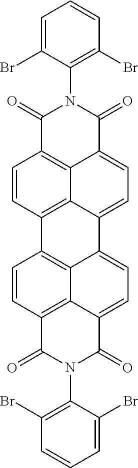 [SMILES: O=C1c2ccc3c4ccc5c6c(ccc(c7ccc(c2c37)C(=O)N1c1c(Br)cccc1Br)c64)C(=O)N(c1c(Br)cccc1Br)C5=O]

In a flame dried 100 mL Schlenk flask 2.0 g (5.10 mmol) of perylene tetracarboxylic acid bis anhydride, 12.8 g (10 eq; 51.0 mmol) of 2,6-dibromo aniline, 940 mg zinc acetate (1 eq, 5.1 mmol) and 70 g imidazole were added. The solids were mixed by shaking and degassed three times. It was heated to 180° C. under argon for 48 h. After cooling the melt to 100° C. water was added. The resulting solution was poured into 1M HCl solution. The precipitate was filtered and successively washed with water and cold methanol. The residue was dissolved in DCM and purified by column chromatography on silica to attain the product in 10% yield (460 mg; 0.54 mmol). On a smaller scale yields of up to 25% can be achieved.

¹H NMR (300 MHz, (CDCl₂)₂, 298K) δ 7.32 (t, J=8.1 Hz, 2H), 7.80 (dd, J=10.7, 8.1 Hz, 4H), 8.79 (d, J=3.0 Hz, 8H).

¹³C NMR (75 MHz, (CDCl₂)₂, 298K) δ 161.87, 145.11, 135.42, 132.61, 132.36, 124.32, 123.72, 122.87, 112.83.

FD mass spectrum (8 kV): m/z (%): calcd for 858.1. found: 857.6 (100) [M]⁺.

### Example 3.2

### Preparation of N,N′-bis(2,6-diacetylphenyl)-perylene-3,4,9,10-tetracarboxybisimide

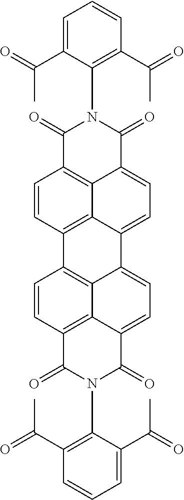 [SMILES: CC(=O)c1cccc(C(C)=O)c1N1C(=O)c2ccc3c4ccc5c6c(ccc(c7ccc(c2c37)C1=O)c64)C(=O)N(c1c(C(C)=O)cccc1C(C)=O)C5=O]

In a dried 500 mL three neck flask equipped with a reflux condenser 320 mg (0.37 mmol) of N,N′-bis(2,6-dibromophenyl)-perylene-3,4,9,10-tetracarboxybisimide from example 3.1 dissolved in 400 mL toluene were purged with argon for 30 minutes and 810 mg (6.0 eq, 2.24 mmol, 0.76 ml) tributyl(1-ethoxyvinyl)tin were added. The reaction mixture was degassed for additional 5 minutes and 175 mg (0.4 eq, 0.15 mmol) of tetrakis(triphenylphosphine)palladium were added. The reaction mixture was then heated to reflux for 48 h with two additional equivalents of tributyl(1-ethoxyvinyl)tin being added after each 12 hours. After completion of the reaction monitored by thin layer chromatography the product was poured onto 1 L of 2M hydrochloric acid and stirred for 12 h. The organic layer was separated and the aqueous layer was extracted three times with DCM. The combined organic layers were dried over sodium sulfate, filtered and evaporated. The crude material was purified by column chromatography on silica with a mixture of DCM:THF (10:1). The product was obtained as violet powder in 57% yield (152 mg; 0.21 mmol).

¹H NMR (300 MHz, (CDCl₂)₂, 298K) δ 2.60 (s, 6H), 7.77 (t, J=7.9 Hz, 1H), 8.10 (d, J=7.8 Hz, 2H), 8.65 (s, 4H).

¹³C NMR (75 MHz, (CDCl₂)₂, 298K) δ 29.24, 99.36, 99.83, 106.25, 109.58, 122.88, 131.73, 132.62, 135.13, 135.98, 141.44, 149.19, 163.65, 187.37.

FD mass spectrum (8 kV): m/z (%): calcd for 710.7. found: 709.3 (100) [M]⁺.

### Example 3.3

### Preparation of peryleno[3′,4′:7,8,9;9′,10′:7,8,9]diquinolizino[3,4,5,6-ija]quinoline-2,9,13,20-tetrone

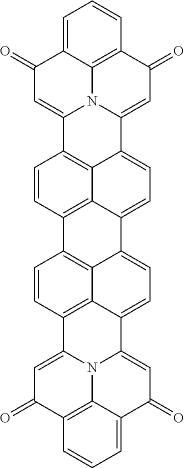 [SMILES: O=c1cc2c3ccc4c5ccc6c7c(ccc(c8ccc(c3c48)c3cc(=O)c4cccc1c4n23)c57)c1cc(=O)c2cccc3c(=O)cc6n1c32]

In a 25 mL Schlenk flask 140 mg (0.20 mmol) of N,N′-bis(2,6-diacetylphenyl)-perylene-3,4,9,10-tetracarboxybisimide from example 3.2 were mixed with 5.5 g imidazole and were degassed three times. Under an argon atmosphere the reaction mixture was heated to 120° C. for 2 h. Water was added to the reaction mixture and the resulting mixture was then poured onto 1M HCl solution. The precipitate was filtered off and successively washed with water, methanol, DCM and THF. The residue was dissolved in sulfuric acid and precipitated by slowly diffusion of water. The precipitate was filtered off, washed with water, methanol and dried in vacuo to give 107 mg (0.17 mmol) of the desired product in 85% yield.

¹H NMR (300 MHz, D₂SO₄) δ 8.07 (t, J=7.9 Hz, 2H), 8.36 (br, 4H), 8.98 (two signals (8.97, 9.00), 12H)

MALDI-TOF: m/z (%): calcd for 638.64. found: 637.20 (100) [M]⁺.

### Example 4

### Example 4.1

### Preparation of N,N′-bis(2,6-dibromophenyl)-1,6,7,12-tetra-tert-octyl-phenoxy-perylene-3,4,9,10-tetracarboxybisimide

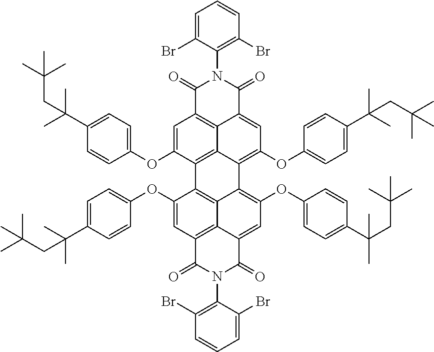 [SMILES: CC(C)(C)CC(C)(C)c1ccc(Oc2cc3c4c(cc(Oc5ccc(C(C)(C)CC(C)(C)C)cc5)c5c6c(Oc7ccc(C(C)(C)CC(C)(C)C)cc7)cc7c8c(cc(Oc9ccc(C(C)(C)CC(C)(C)C)cc9)c(c2c45)c86)C(=O)N(c2c(Br)cccc2Br)C7=O)C(=O)N(c2c(Br)cccc2Br)C3=O)cc1]

In a flame dried 25 mL Schlenk flask 0.4 g (0.33 mmol) of 1,6,7,12-tetra-tert-octyl-phenoxy perylene tetracarboxylic acid bis anhydride, 1.66 g (20 eq; 6.6 mmol) of 2,6-dibromo aniline, 0.21 g (2 eq; 0.66 mmol) zinc acetate and 5 g imidazole were added, mixed by careful shaking and degassed three times. The reaction mixture was heated to 160° C. under argon and exclusion of light for 12 h. After cooling to 100° C. water was added to the melt and the resulting suspension was poured into 1M hydrochloric acid. The precipitate was filtered and washed with water and methanol. The residue was purified by column chromatography on silica (PE/DCM) to attain 305 mg (0.18 mmol) product in 55% yield.

¹H NMR (300 MHz, CD₂Cl₂, 298K) δ 0.76 (s, 36H), 1.36 (s, 24H), 1.74 (s, 8H), 6.91-6.96 (m, 8H), 7.25 (t, J=8.20 Hz, 2H), 7.31-7.36 (m, 8H), 7.71 (d, J=7.65 Hz, 4H), 8.15 (s, 4H) ppm.

¹³C NMR (75 MHz, CD₂Cl₂, 298K) δ 31.93, 32.16, 32.85, 38.86, 57.49, 119.98, 120.26, 120.46, 121.16, 122.73, 124.79, 128.42, 131.96, 133.02, 135.48, 147.62, 153.00, 156.94, 162.22 ppm.

HRMS (ESI⁺): calcd for C₉₂H₉₅N₂O₈Br₄ [M+H]+ 1671.3822. found: 1671.3776.

UV-Vis (CH₂Cl₂): λₘₐₓ=590 nm (50500 m⁻¹cm⁻¹).

### Example 4.2

### N,N′-bis(2,6-diacetylphenyl)-1,6,7,12-tetra-tert-octyl-phenoxy-perylene-3,4,9,10-tetracarboxybisimide

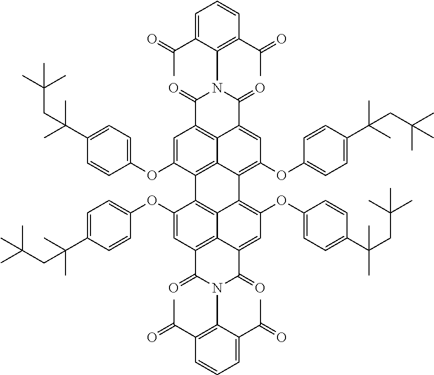 [SMILES: CC(=O)c1cccc(C(C)=O)c1N1C(=O)c2cc(Oc3ccc(C(C)(C)CC(C)(C)C)cc3)c3c4c(Oc5ccc(C(C)(C)CC(C)(C)C)cc5)cc5c6c(cc(Oc7ccc(C(C)(C)CC(C)(C)C)cc7)c(c7c(Oc8ccc(C(C)(C)CC(C)(C)C)cc8)cc(c2c37)C1=O)c64)C(=O)N(c1c(C(C)=O)cccc1C(C)=O)C5=O]

In a dry 250 mL Schlenk flask 0.25 g (0.15 mmol) of the compound obtained in example 4.1 were dissolved in 30 mL dry dioxane and thoroughly degassed by purging with argon for 30 min. Then 0.3 mL tributyl(1-ethoxyvinyl)tin (6 eq, 0.9 mmol) were added and the system was purged for additional 5 minutes. Under argon atmosphere 0.4 eq tetrakis(triphenylphosphine)palladium (69 mg; 0.6 mmol) were added and the reaction mixture was heated to 100° C. for 24 hours. Additional 0.3 mL tributyl(1-ethoxy-vinyl)tin (6 eq, 0.9 mmol) were added and stirring was continued for 24 hours. After cooling to room temperature 200 mL 2 m HCl solution was added and the resulting mixture was vigorously stirred for further 6 hours. The reaction mixture was first extracted with DCM and the combined organic layers were washed with brine and dried with sodium sulfate. After evaporation the residue was purified by column chromatography (silica, DCM). Upon precipitation from methanol the product was obtained in form of red crystals in 76% yield (173 mg; 0.113 mmol).

¹H NMR (300 MHz, CD₂Cl₂, 298K) δ 0.75 (s, 36H), 1.35 (s, 24H), 1.73 (s, 8H), 2.47 (s, 12H), 6.93 (d, J=8.7 Hz, 8H), 7.31 (d, J=8.8 Hz, 8H), 7.67 (t, J=8.00 Hz, 2H), 7.99 (d, J=7.70 Hz, 4H), 8.04 (s, 4H) ppm.

¹³C NMR (75 MHz, CD₂Cl₂, 298K) δ 197.56, 162.73, 155.55, 151.87, 145.95, 136.86, 131.48, 130.75, 128.24, 126.97, 121.66, 119.78, 119.03, 118.34, 56.18, 37.49, 31.49, 30.81, 30.57, 28.22 ppm.

HRMS (ESI⁺): calcd for C₁₀₀H₁₀₇N₂O₁₂ [M+H]+ 1527.7824. found: 1527.7814.

UV-Vis (CH₂Cl₂): λₘₐₓ=588 nm (53700 m⁻¹cm⁻¹).

### Example 4.3

### 5,6,16,17-tetra-tert-octyl-phenoxy-peryleno[3′,4′:7,8,9;9′,10′:7,8,9]diquinolizino[3,4,5,6-ija]quinoline-2,9,13,20-tetrone

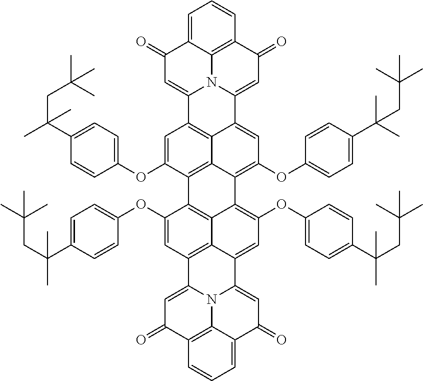 [SMILES: CC(C)(C)CC(C)(C)c1ccc(Oc2cc3c4c(cc(Oc5ccc(C(C)(C)CC(C)(C)C)cc5)c5c6c(Oc7ccc(C(C)(C)CC(C)(C)C)cc7)cc7c8c(cc(Oc9ccc(C(C)(C)CC(C)(C)C)cc9)c(c2c54)c68)c2cc(=O)c4cccc5c(=O)cc7n2c45)c2cc(=O)c4cccc5c(=O)cc3n2c54)cc1]

A 50 mL round bottom flask was charged with 150 mg (0.1 mmol) of N,N′-bis(2,6-diacetylphenyl)-1,6,7,12-tetra-tert-octyl-phenoxy-perylene-3,4,9,10-tetracarboxy-bisimide from example 4.2, 5 g imidazole and 5 mL methanol and purged with argon. The reaction mixture was heated to 120° C. under stirring and methanol was allowed to evaporate through a canula. After four hours the starting material was completely reacted and 30 mL of 1 m hydrochloric acid were added. The precipitate was filtered off and washed with water and methanol. The crude product was purified by column chromatography on silica (toluene/THF=20:1) to attain pure product as a green solid in 85% yield (121 mg; 0.083 mmol).

¹H NMR (300 MHz, CD₂Cl₂, 298K) δ 0.74 (s, 36H), 1.33 (s, 24H), 1.70 (s, 8H), 6.84 (d, J=8.7 Hz, 8H), 6.92 (s, 4H), 7.29 (d, J=8.8 Hz, 8H), 7.57 (t, J=7.6 Hz, 2H), 7.89 (s, 4H), 8.54 (d, J=7.6 Hz, 4H) ppm.

¹³C NMR (75 MHz, CD₂Cl₂, 298K) δ 49.58, 51.70, 52.02, 52.72, 58.70, 130.72, 139.66, 139.66, 143.58, 146.80, 148.33, 151.73, 173.27, 176.66, 196.74 ppm.

HRMS (ESI⁺): calcd for C100H99N2O8 [M+H]⁺ 1455.7401. found: 1455.7407.

UV-Vis (CH₂Cl₂): λₘₐₓ=700 nm m⁻¹cm⁻¹ (69000 m⁻¹cm⁻¹), 453 (20800 m⁻¹cm⁻¹).

### Example 5

### Example 5.1

### Preparation of 2-(2,6-dibromophenyl)isoindoline-1,3-dione

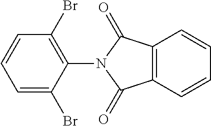 [SMILES: O=C1c2ccccc2C(=O)N1c1c(Br)cccc1Br]

The title compound was prepared in analogy to the method described in example 2.1.

Yield: 36%.

¹H NMR (300 MHz, CD₂Cl₂, 298K): δ 7.28 (t, J=8.1 Hz, 1H), 7.73 (d, J=8.1 Hz, 2H), 7.86 (dd, J=5.5, 3.1 Hz, 2H), 7.99 (dd, J=5.5, 3.0 Hz, 2H);

¹³C NMR (75 MHz, CD₂Cl₂, 298K): δ 124.57, 125.66, 131.93, 132.34, 132.69, 133.07, 135.31, 166.12 (2C, C═O).

FD mass spectrum (8 kV): m/z (%): calcd for C₁₄H₇Br₂NO₂: 381.02. found: 381.5 (100) [M]⁺.

### Example 5.2

### Preparation of 2-(2,6-diacetylphenyl)isoindoline-1,3-dione

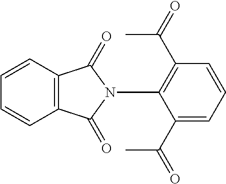 [SMILES: CC(=O)c1cccc(C(C)=O)c1N1C(=O)c2ccccc2C1=O]

The title compound was prepared in analogy to the method described in example 2.2.

Yield: 58%.

¹H NMR (300 MHz, CD₂Cl₂, 298K) δ 2.54 (s, 6H), 7.68 (m, 1H), 7.81 (dd, J=5.6, 3.0 Hz, 2H), 7.90 (dd, J=5.4, 3.2 Hz, 2H), 7.95 (d, J=7.8 Hz, 2H)

¹³C NMR (75 MHz, CDCl₃, 298K) δ 29.17 (2C, CH₃), 124.07, 127.02, 129.61, 132.02, 132.51, 134.86, 138.83, 167.76 (2C, C═O), 199.28 (2C, C═O).

FD mass spectrum (8 kV): m/z (%): calcd for C₁₈H₁₃NO₄: 307.30. found: 307.9 (100) [M]⁺.

### Example 5.3

### Preparation of benzo[1,2]indolizino[6,5,4,3-ija]quinoline-4,11-dione

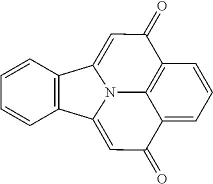 [SMILES: O=c1cc2c3ccccc3c3cc(=O)c4cccc1c4n23]

The title compound was prepared in analogy to the method described in example 2.3, route (b). Yield: 88%.

¹H NMR (500 MHz, C₂D₂Cl₄, 373K) δ 6.78 (s, 2H), 7.61 (t, J=7.7 Hz, 1H), 7.67 (dd, J=5.7, 3.0 Hz, 2H), 7.88 (dd, J=5.6, 3.1 Hz, 2H), 8.49 (d, J=7.7 Hz, 2H).

¹³C NMR (126 MHz, C₂D₂Cl₄, 373K) δ 106.99 (2C, CH), 123.29, 124.06, 125.62, 132.38, 132.59, 132.71, 135.16 (1C, C—N), 145.74 (2C, C═N), 179.27 (2C, C═O).

FD mass spectrum (8 kV): m/z (%): calcd for C₁₈H₉NO₂:271.27. found: 271.6 (100) [M]⁺.

UV-Vis (CH₂Cl₂): λₘₐₓ=442, 414 nm (26680, 14480 M⁻¹cm⁻¹).

### Example 5.4

### Preparation of 2,2′-(benzo[1,2]indolizino[6,5,4,3-ija]quinoline-4,11-diylidene)dimalononitrile

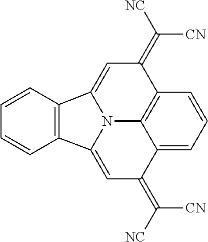 [SMILES: N#CC(C#N)=c1cc2c3ccccc3c3cc(=C(C#N)C#N)c4cccc1c4n23]

The title compound was prepared in analogy to the method described in example 2.4, route (b). Yield: 27%.

¹H NMR (500 MHz, C₂D₂Cl₂, 403K) δ 7.61 (s, 2H), 7.82 (dd, J=5.6, 3.0 Hz, 2H), 7.73-7.89 (m, 1H), 8.09 (dd, J=5.6, 3.0 Hz, 2H), 9.42 (d, J=8.3 Hz, 2H).

¹³C NMR (126 MHz, C₂D₂Cl₄, 403K) δ 69.20 (2C, C(CN)₂), 105.66 (2C, CH), 114.68, 119.99, 123.13, 126.48, 127.76, 131.33, 132.11, 132.82, 140.22, 150.89.

HRMS: m/z (%): calcd for C₂₄H₉N₅: 367.0858 (100). found: 367.0840 (100) [M]⁺.

FD mass spectrum (8 kV): m/z (%): calcd for C₂₄H₉N₅: 367.37. found: 367.5 (100) [M]⁺.

UV-Vis (CH₂Cl₂): λₘₐₓ=625, 576 nm (56850, 30770 M⁻¹cm⁻¹).

### Example 6

### Example 6.1

2-(2,6-dibromo-4-methylphenyl)isoindoline-1,3-dione

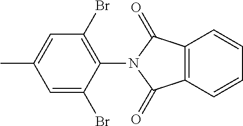 [SMILES: Cc1cc(Br)c(N2C(=O)c3ccccc3C2=O)c(Br)c1]

The title compound was prepared in analogy to the method describe in example 2.1.

Yield: 80%.

¹H NMR (300 MHz, CD₂Cl₂, 298K): δ 2.40 (s, 3H), 7.55 (s, 2H), 7.85 (dd, J=5.5, 3.0 Hz, 2H), 7.97 (dd, J=5.5, 3.1 Hz, 2H).

¹³C NMR (75 MHz, CD₂Cl₂, 298K): δ 21.15 (1C, CH₃), 124.50, 124.91, 128.95, 132.35, 133.61, 135.24, 144.02, 166.31 (2C, C═O).

FD mass spectrum (8 kV): m/z (%): calcd for C₁₅H₉Br₂NO₂: 395.02. found: 395.8 (100) [M]⁺.

### Example 6.2

### Preparation of 2-(2,6-diacetyl-4-methylphenyl)isoindoline-1,3-dione

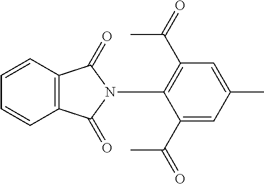 [SMILES: CC(=O)c1cc(C)cc(C(C)=O)c1N1C(=O)c2ccccc2C1=O]

The title compound was prepared in analogy to the method describe in example 2.2.

Yield: 75%.

¹H NMR (300 MHz, CD₂Cl₂, 298K) δ 2.51 (s, 5H), 2.53 (s, 3H), 7.76 (s, 2H), 7.80 (dd, J=5.4, 3.2 Hz, 2H), 7.89 (dd, J=5.6, 3.0 Hz, 2H).

¹³C NMR (75 MHz, CDCl₃, 298K) δ 21.56 (1C, CH₃), 29.36 (2C, CH₃), 124.18, 124.62, 132.74, 132.86, 134.97, 138.78, 140.50, 168.11 (2C, C═O), 199.58 (2C, C═O).

FD mass spectrum (8 kV): m/z (%): calcd for C₁₉H₁₅NO₄: 321.33. found: 321.0 (100) [M]⁺.

### Example 6.3

### Preparation of 2-methylbenzo[1,2]indolizino[6,5,4,3-ija]quinoline-4,11-dione

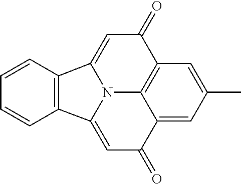 [SMILES: Cc1cc2c(=O)cc3c4ccccc4c4cc(=O)c(c1)c2n34]

The title compound was prepared in analogy to the method describe in example 2.3, route (b). Yield: 79%.

¹H NMR (500 MHz, C₂D₂Cl₄, 373K) δ 2.61 (s, 3H), 6.74 (s, 2H), 7.66 (dd, J=5.4, 3.0 Hz, 2H), 7.87 (m, 2H), 8.33 (s, 2H).

¹³C NMR (126 MHz, C₂D₂Cl₄, 373K) δ 20.98 (1C, CH₃), 106.62 (2C, CH), 122.91, 124.22, 131.89, 132.56, 133.07, 133.49, 135.90, 145.23, 178.81 (2C, C═O).

FD mass spectrum (8 kV): m/z (%): calcd for for C₁₉H₁₁NO₂: 285.30. found: 285.8 (100) [M]⁺.

UV-Vis (CH₂Cl₂): λₘₐₓ=450, 435 and 422 nm (25380, 14350 and 13100 M⁻¹cm⁻¹).

### Example 6.4

### Preparation of 2,2′-(2-methylbenzo[1,2]indolizino[6,5,4,3-ija]quinoline-4,11-diylidene)dimalononitrile

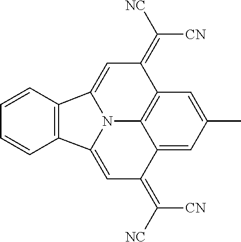 [SMILES: Cc1cc2c(=C(C#N)C#N)cc3c4ccccc4c4cc(=C(C#N)C#N)c(c1)c2n34]

The title compound was prepared in analogy to the method describe in example 2.4, route (b). Yield: 26%.

¹H NMR (300 MHz, CD₂Cl₂, 298K) 2.64 (s, 3H), 7.52 (s, 2H), 7.76 (dd, J=5.8, 3.0 Hz, 2H), 8.03 (dd, J=5.8, 3.0 Hz, 2H), 9.16 (s, 2H).

HRMS: m/z (%): calcd for C₂₅H₁₁N₅: 381.1014 (100). found: 381.0977 (100) [M]⁺.

FD mass spectrum (8 kV): m/z (%): calcd for C₂₅H₁₁N₅: 381.40. found: 381.6 (100) [M]⁺.

UV-Vis (CH₂Cl₂): λₘₐₓ=630, 581 nm.

### Example 7

### Example 7.1

### Preparation of 2-(2,6-dibromo-4-propylphenyl)isoindoline-1,3-dione

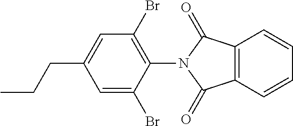 [SMILES: CCCc1cc(Br)c(N2C(=O)c3ccccc3C2=O)c(Br)c1]

The title compound was prepared in analogy to the method describe in example 2.1.

Yield: 43%.

¹H NMR (300 MHz, CD₂Cl₂, 298K): δ 0.99 (t, J=7.3 Hz, 3H), 1.67 (m, 2H), 2.63 (m, 2H), 7.55 (s, 2H), 7.85 (dd, J=5.5, 3.1 Hz, 2H), 7.98 (dd, J=5.5, 3.1 Hz, 2H).

¹³C NMR (75 MHz, CD₂Cl₂, 298K): δ 14.06 (1C, CH₃), 24.60 (1C, CH₂), 37.73 (1C, CH₂), 124.51, 124.99, 132.37, 133.03, 135.24, 148.61, 166.32 (2C, C═O).

FD mass spectrum (8 kV): m/z (%): calcd for C₁₇H₁₃Br₂NO₂: 423.10. found: 423.6 (100) [M]⁺.

### Example 7.2

### Preparation of 2-(2,6-diacetyl-4-propylphenyl)isoindoline-1,3-dione

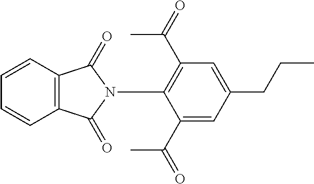 [SMILES: CCCc1cc(C(C)=O)c(N2C(=O)c3ccccc3C2=O)c(C(C)=O)c1]

The title compound was prepared in analogy to the method describe in example 2.2.

Yield 54%.

¹H NMR (300 MHz, CD₂Cl₂, 298K) δ 1.03 (t, J=7.3 Hz, 3H), 1.75 (h, J=7.3 Hz, 2H), 2.77 (m, 2H), 7.75 (s, 2H), 7.80 (m, 2H), 7.89 (m, 2H).

¹³C NMR (75 MHz, CDCl₃, 298K) δ 14.13 (1C, CH₃), 24.93 (1C, CH₂), 29.37 (2C, CH₃), 38.14 (1C, CH₃), 124.20, 124.29, 124.80, 132.25, 132.75, 134.97, 138.86, 145.11, 168.12 (2C, C═O), 199.67 (2C, C═O).

FD mass spectrum (8 kV): m/z (%): calcd for C₂₁H₁₉NO₄: 349.39. found: 349.9 (100) [M]⁺.

### Example 7.3

### Preparation of 2-propylbenzo[1,2]indolizino[6,5,4,3-ija]quinoline-4,11-dione

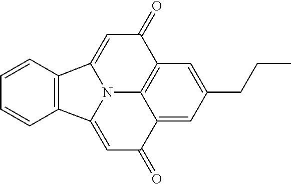 [SMILES: CCCc1cc2c(=O)cc3c4ccccc4c4cc(=O)c(c1)c2n34]

The title compound was prepared in analogy to the method described in example 2.3, route (b). Yield: 72%.

¹H NMR (300 MHz, CD₂Cl₂, 298K) δ 0.98 (t, J=7.3 Hz, 3H), 1.74 (m, 2H), 2.79 (m, 2H), 6.68 (s, 2H), 7.62 (dd, J=5.7, 3.1 Hz, 2H), 7.84 (dd, J=5.7, 3.1 Hz, 2H), 8.26 (s, 2H).

¹³C NMR (75 MHz, CD₂Cl₂, 298K) δ 13.96 (1C, CH₃), 25.10 (1C, CH₂), 38.25 (1C, CH₂), 107.40 (2C, CH), 123.82, 124.87, 132.70, 133.72, 134.38, 141.44, 146.16, 179.86 (2C, C═O).

FD mass spectrum (8 kV): m/z (%): calcd for C₂₁H₁₅NO₂: 313.36. found: 313.5 (100) [M]⁺.

UV-Vis (CH₂Cl₂): λₘₐₓ=450, 435 and 422 nm (27290, 16080 and 14440 M⁻¹cm⁻¹).

### Example 7.4

### Preparation of 2,2′-(2-propylbenzo[1,2]indolizino[6,5,4,3-ija]quinoline-4,11-diylidene)dimalononitril

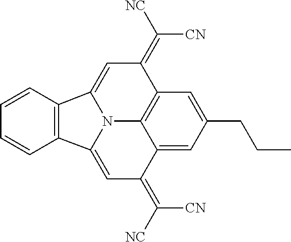 [SMILES: CCCc1cc2c(=C(C#N)C#N)cc3c4ccccc4c4cc(=C(C#N)C#N)c(c1)c2n34]

The title compound was prepared in analogy to the method describe in example 2.4, route (b). Yield: 38%.

¹H NMR (300 MHz, CD₂Cl₂, 298K) δ 0.75-0.98 (m, 3H), 1.55-1.69 (m, 2H), 2.34-2.53 (m, 2H), 7.99-8.13 (m, 2H), 9.28 (s, 1H), 8.00-8.14 (m, 1H), 7.57 (s, 1H), 7.72-7.81 (m, 1H).

HRMS: m/z (%): calcd for C₂₇H₁₅N₅: 409.1327 (100). found: 409.1306 (100) [M]⁺.

FD mass spectrum (8 kV): m/z (%): calcd for C₂₇H₁₅N₅: 409.45. found: 409.8 (100) [M]⁺.

UV-Vis (CH₂Cl₂): λₘₐₓ=630, 580 nm (81120, 39980 M⁻¹cm⁻¹)

### Example 8

### Example 8.1

### Preparation of 2-(2,6-dibromo-4-isopropylphenyl)isoindoline-1,3-dione

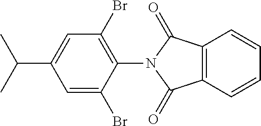 [SMILES: CC(C)c1cc(Br)c(N2C(=O)c3ccccc3C2=O)c(Br)c1]

The title compound was prepared in analogy to the method describe in example 2.1.

Yield: 64%.

¹H NMR (300 MHz, CD₂Cl₂, 298K): δ 1.29 (d, J=6.9 Hz, 5H), 2.96 (hept, J=6.9 Hz, 1H), 7.59 (s, 2H), 7.85 (dd, J=5.5, 3.0 Hz, 2H), 7.98 (dd, J=5.5, 3.1 Hz, 2H).

¹³C NMR (75 MHz, CD₂Cl₂, 298K): δ 23.84 (2C, CH₃), 34.48 (1C, CH), 124.51, 125.13, 129.12, 131.33, 132.36, 135.25, 154.55, 166.34 (2C, C═O).

FD mass spectrum (8 kV): m/z (%): calcd for C₁₇H₁₃Br₂NO₂: 423.10. found: 423.2 (100) [M]⁺.

### Example 8.2

### Preparation of 2-(2,6-diacetyl-4-isopropylphenyl)isoindoline-1,3-dione

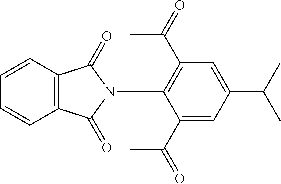 [SMILES: CC(=O)c1cc(C(C)C)cc(C(C)=O)c1N1C(=O)c2ccccc2C1=O]

The title compound was prepared in analogy to the method describe in example 2.2.

Yield 54%.

¹H NMR (300 MHz, CD₂Cl₂, 298K) δ 1.36 (d, J=6.9 Hz, 6H), 2.53 (s, 6H), 3.10 (hept, J=6.8 Hz, 1H), 7.80 (m, 4H), 7.90 (m, 2H).

¹³C NMR (75 MHz, CDCl₃, 298K) δ 24.00 (2C, CH₃), 29.38 (2C, CH₃), 34.66 (1C, CH), 124.21, 124.80, 130.36, 132.73, 134.99, 139.03, 151.06, 168.14 (2C, C═O), 199.76 (2C, C═O).

FD mass spectrum (8 kV): m/z (%): calcd for C₂₁H₁₉NO₄: 349.39. found: 349.2 (100) [M]⁺.

### Example 8.3

### Preparation of 2-isopropylbenzo[1,2]indolizino[6,5,4,3-ija]quinoline-4,11-dione

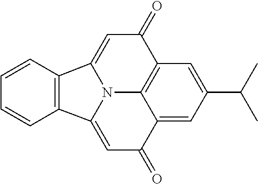 [SMILES: CC(C)c1cc2c(=O)cc3c4ccccc4c4cc(=O)c(c1)c2n34]

The title compound was prepared in analogy to the method described in example 2.3, route (b). Yield: 72%.

¹H NMR (300 MHz, CD₂Cl₂, 298K) δ 1.36 (d, J=6.9 Hz, 6H), 3.14 (hept, J=6.8 Hz, 1H), 6.64 (s, 2H), 7.59 (dd, J=5.7, 3.1 Hz, 2H), 7.80 (dd, J=5.7, 3.1 Hz, 2H), 8.30 (s, 2H).

¹³C NMR (75 MHz, CD₂Cl₂, 298K) δ 24.20 (2C, CH₃), 34.81 (1C, CH), 107.35, 123.74, 124.86, 130.87, 132.66, 133.60, 134.30, 146.02, 147.56, 179.76 (2C, C═O).

FD mass spectrum (8 kV): m/z (%): calcd for C₂₁H₁₅NO₂: 313.36. found: 313.7 (100) [M]⁺.

UV-Vis (CH₂Cl₂): λₘₐₓ=450, 434 and 422 nm (27300, 16100 and 14450 M⁻¹cm⁻¹)

### Example 8.4

### Preparation of 2,2′-(2-isopropylbenzo[1,2]indolizino[6,5,4,3-ija]quinoline-4,11-diylidene)dimalononitrile

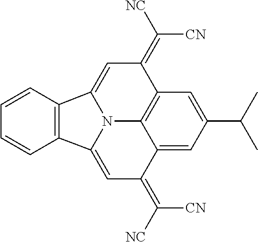 [SMILES: CC(C)c1cc2c(=C(C#N)C#N)cc3c4ccccc4c4cc(=C(C#N)C#N)c(c1)c2n34]

The title compound was prepared in analogy to the method described in example 2.4, route (b). Yield: 48%

¹H NMR (500 MHz, C₂D₂Cl₄, 353K) δ 1.48 (d, J=6.0 Hz, 6H), 3.25 (hept, J=6.7 Hz, 1H), 7.57 (s, 2H), 7.79 (dd, J=5.8, 3.1 Hz, 2H), 8.06 (dd, J=5.7, 3.1 Hz, 2H), 9.32 (s, 2H).

HRMS: m/z (%): calcd for C₂₇H₁₅N₅: 409.1327 (100). found: 409.1298 (100) [M]⁺.

FD mass spectrum (8 kV): m/z (%): calcd for C₂₇H₁₅N₅: 409.45. found: 409.5 (100) [M]⁺.

UV-Vis (CH₂Cl₂): λₘₐₓ=630, 580 nm (81740, 40580 M⁻¹cm⁻¹).

### Example 9

### Example 9.1

### Preparation of 2-(2,6-dibromo-4-(trifluoromethyl)phenyl)isoindoline-1,3-dione

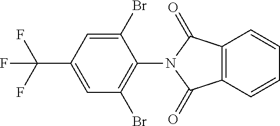 [SMILES: O=C1c2ccccc2C(=O)N1c1c(Br)cc(C(F)(F)F)cc1Br]

The title compound was prepared in analogy to the method describe in example 2.1 and purified by column chromatography (CH₂Cl₂/hexane). Yield: 61%.

¹H NMR (300 MHz, CDCl₃, 298K) δ 7.88 (dd, J=5.5, 3.1 Hz, 2H), 8.00 (m, 4H).

¹³C NMR (75 MHz, CDCl₃, 298K) δ 120.82, 124.44, 124.78, 126.45, 130.18 (q, J=3.6 Hz, 1C, CF₃), 132.23, 134.13, 134.59, 135.53, 135.73, 165.60 (2C, C═O).

FD mass spectrum (8 kV): m/z (%): calcd for C₁₅H₆Br₂F₃NO₂: 449.02. found: 449.7 (100) [M]⁺.

### Example 9.2

### Preparation of 2-(2,6-diacetyl-4-(trifluoromethyl)phenyl)isoindoline-1,3-dione

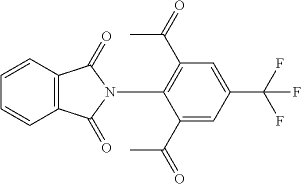 [SMILES: CC(=O)c1cc(C(F)(F)F)cc(C(C)=O)c1N1C(=O)c2ccccc2C1=O]

The title compound was prepared in analogy to the method describe in example 2.2.

Yield: 61%.

¹H NMR (300 MHz, CD₂Cl₂, 298K) b 2.59 (s, 6H), 7.84 (m, 2H), 7.92 (m, 2H), 8.15 (s, 2H).

¹³C NMR (75 MHz, CDCl₃, 298K) δ 29.18 (2C, CH₃), 121.80, 124.56, 125.42, 128.48 (q, J=3.7 Hz, 1C, CF₃), 130.24, 130.88, 131.33, 131.78, 132.45, 135.40, 139.93, 167.47 (2C, C═O), 198.38 (2C, C═O).

FD mass spectrum (8 kV): m/z (%): calcd for C₁₉H₁₂F₃NO₄: 375.30. found: 375.5 (100) [M]⁺.

### Example 9.3

### Preparation of 2-(trifluoromethyl)benzo[1,2]indolizino[6,5,4,3-ija]quinoline-4,11-dione

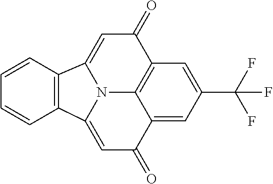 [SMILES: O=c1cc2c3ccccc3c3cc(=O)c4cc(C(F)(F)F)cc1c4n23]

the title compound was prepared in analogy to the method described in example 2.3, route (b). Yield: 29%.

¹H NMR (500 MHz, C₂D₂Cl₄, 373K) δ 6.84 (s, 2H), 7.74 (dd, J=5.7, 3.0 Hz, 2H), 7.94 (dd, J=5.6, 3.1 Hz, 2H), 8.77 (s, 2H).

¹³C NMR (126 MHz, C₂D₂Cl₄, 373K) b 106.97 (2C, CH), 123.27, 124.67, 129.02 (q, J=3.3 Hz, CF₃), 131.86, 131.94, 132.50, 132.72, 136.83, 146.00, 177.73 (2C, C═O).

FD mass spectrum (8 kV): m/z (%): calcd for C₁₉H₈F₃NO₂: 339.27. found: 339.5 (100) [M]⁺.

UV-Vis (CH₂Cl₂): λₘₐₓ=440, 414 nm

### Example 10

### Example 10.1

### Preparation of 2-(2,6-dibromo-4-propylphenyl)-4,5,6,7-tetrafluoroisoindoline-1,3-dione

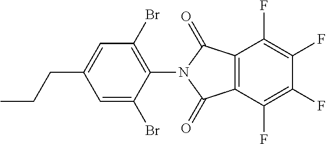 [SMILES: CCCc1cc(Br)c(N2C(=O)c3c(F)c(F)c(F)c(F)c3C2=O)c(Br)c1]

The title compound was prepared in analogy to the method describe in example 2.1 and purified by column chromatography (CH₂Cl₂/hexane). Yield: 61%.

¹H NMR (300 MHz, CDCl₃, 298K) δ 0.99 (t, J=7.3 Hz, 3H), 1.68 (dt, J=14.8, 7.4 Hz, 2H), 2.56-2.73 (m, 2H), 7.56 (s, 2H).

¹³C NMR (75 MHz, CDCl₃, 298K) δ 14.04 (1C, CH₃), 24.56 (1C, CH₂), 37.77 (1C, CH₂), 114.27, 124.69, 127.65, 133.20, 144.21, 146.35, 149.41, 160.45 (2C, C═O).

FD mass spectrum (8 kV): m/z (%): calc. for C₁₇H₉Br₂F₄NO₂: 495.07. found: 495.7 (100) [M]⁺.

### Example 10.2

### Preparation of 2-(2,6-diacetyl-4-propylphenyl)-4,5,6,7-tetrafluoroisoindoline-1,3-dione

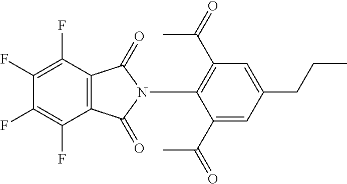 [SMILES: CCCc1cc(C(C)=O)c(N2C(=O)c3c(F)c(F)c(F)c(F)c3C2=O)c(C(C)=O)c1]

The title compound was prepared in analogy to the method describe in example 2.2.

Yield: 56%.

¹H NMR (300 MHz, CD₂Cl₂, 298K) δ 1.04 (t, J=7.3 Hz, 3H), 1.76 (h, J=7.4 Hz, 2H), 2.55 (s, 6H), 2.79 (t, J=7.4 Hz, 2H), 7.83 (s, 2H).

FD mass spectrum (8 kV): m/z (%): calc for C₂₁H₁₅F₄NO₄: 421.35. found: 421.1 (100) [M]⁺.

### Example 11

### Example 11.1

### Preparation of 2,6-bis(2,6-dibromo-4-propylphenyl)pyrrolo[3,4-f]isoindole-1,3,5,7(2H,6H)-tetraone

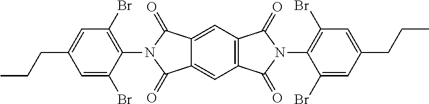 [SMILES: CCCc1cc(Br)c(-n2c(=O)c3cc4c(=O)n(-c5c(Br)cc(CCC)cc5Br)c(=O)c4cc3c2=O)c(Br)c1]

A mixture of pyromellitic diaanhydride (2.20 g, 10.06 mmol) and 2,6-dibromo-4-n-propylaniline (25.00 mmol) was dissolved in 30 mL of acetic acid and refluxed for 12 h. The reaction was monitored by TLC (petroleum ether/ethyl acetate, 5/1) and upon completion the solvent was evaporated under reduced pressure, the residue was recrystallized from methanol to give the title compound as colorless crystalls.

Yield: 5.80 g (75%).

¹H NMR (300 MHz, C₂D₂Cl₄, 298K): δ 1.01 (t, J=7.3 Hz, 6H), 1.70 (h, J=7.3 Hz, 4H), 2.64 (m, 4H), 7.57 (s, 4H), 8.57 (s, 2H).

¹³C NMR (75 MHz, C₂D₂Cl₄, 298K): δ 13.76 (2C, CH₃), 23.96 (2C, CH₂), 37.14 (2C, CH₂), 120.10, 124.04, 127.33, 132.38, 136.90, 148.51, 163.44 (4C, C═O).

FD mass spectrum (8 kV): m/z (%): calcd for C₂₈H₂₀Br₄N₂O₄: 768.09. found: 768.8 (100) [M]⁺.

### Example 11.2

### Preparation of 2,6-bis(2,6-diacetyl-4-propylphenyl)pyrrolo[3,4-f]isoindole-1,3,5,7(2H,6H)-tetraone

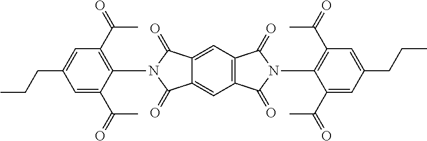 [SMILES: CCCc1cc(C(C)=O)c(-n2c(=O)c3cc4c(=O)n(-c5c(C(C)=O)cc(CCC)cc5C(C)=O)c(=O)c4cc3c2=O)c(C(C)=O)c1]

A solution of the compound from example 11.1 (3.0 mmol), Pd(PPh₃)₄ (367 mg, 0.03 mmol), a in 1,4-dioxane (50 mL) was stirred (10 min) under a positive flow of argon. To the yellow solution was added tributyl(1-ethoxy-1-ethenyl)stannane (6.00 ml, 17.8 mmol) dissolved in 1,4-dioxane (10 mL). The solution was heated at reflux (40 h) whereupon a dark brown solution was formed. The progress of the reaction was monitored to completion using TLC (hexane/DCM). The reaction mixture was cooled to ambient temperature, 50 mL 1N HCl were added and stirred for 5 h at room temperature. The reaction mixture was extracted with dichlorometahne and dried under MgSO₄ and solvent was evaporated until dryness. The crude product was purified by recrystallization from methanol-dichloromethane or, if it was necessary, by column chromatography (hexane/dichloromethane) on silica gel to give product as colorless crystals. Yield: 1.52 g (79%).

¹H NMR (300 MHz, CD₂Cl₂, 298K): δ 1.04 (t, J=7.3 Hz, 6H), 1.77 (h, J=7.3 Hz, 4H), 2.56 (s, 12H), 2.80 (m, 4H), 7.83 (s, 4H), 8.35 (s, 2H).

¹³C NMR (75 MHz, CD₂Cl₂, 298K): δ 14.12 (2C, CH₃), 24.93 (2C, CH₂), 29.39 (4C, CH₃), 38.18 (2C, CH₂), 119.63, 124.37, 132.83, 138.06, 138.28, 145.80, 166.17 (4C, C═O), 199.29 (4C, C═O).

FD mass spectrum (8 kV): m/z (%): calc. for C₃₆H₃₂N₂O₈: 620.66. found: 620.8 (100) [M]⁺

### Example 11.3

### Preparation of

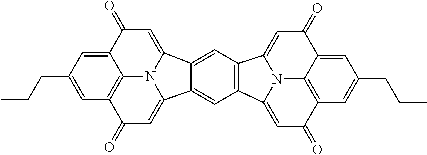 [SMILES: CCCc1cc2c(=O)cc3c4cc5c(cc4c4cc(=O)c(c1)c2n34)c1cc(=O)c2cc(CCC)cc3c(=O)cc5n1c32]

The compound of example 11.2 (300 mg) and imidazole (2-3 g) was stirred at 120° C. for 2 h. The reaction mixture was cooled to ambient temperature, and then 20 mL of water were added and stirred for 30 min at room temperature. The yellow-brown suspension was filtrated and washed several times with small portions of water, methanol and acetone.

Yield: 0.15 g (56%).

¹H NMR (300 MHz, D₂SO₄, 298K): δ 0.44 (t, J=7.6 Hz, 6H), 1.23 (d, J=7.7 Hz, 4H), 2.39 (s, 4H), 7.53 (s, 4H), 8.29 (s, 2H), 8.52 (s, 4H).

¹³C NMR (75 MHz, D₂SO₄, 298K): δ 15.62 (2C, CH₃), 26.32 (2C, CH₂), 40.17 (4C, CH₂), 113.36, 121.81, 135.69, 141.30, 149.32, 149.36, 153.48, 154.39, 183.52 (4C, C═O).

FD mass spectrum (8 kV): m/z (%): calc. for C₃₆H₂₄N₂O₄: 548.60. found: 548.6 (100) [M]⁺

### Example 12

### Example 12.1

### Preparation of 2,6-bis(2,6-dibromo-4-(tert-butyl)phenyl)pyrrolo[3,4-f]isoindole-1,3,5,7(2H,6H)-tetraone

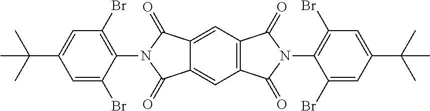 [SMILES: CC(C)(C)c1cc(Br)c(-n2c(=O)c3cc4c(=O)n(-c5c(Br)cc(C(C)(C)C)cc5Br)c(=O)c4cc3c2=O)c(Br)c1]

The title compound was prepared in analogy to the method described in example 11.1.

Yield: 75%.

¹H NMR (500 MHz, C₂D₂Cl₄, 373K) δ 1.44 (s, 18H), 7.77 (s, 4H), 8.59 (s, 2H).

¹³C NMR (126 MHz, C₂D₂Cl₄, 373K): δ 30.74 (6C, CH₃), 34.99 (2C, C—CH₃), 119.66, 124.13, 127.66, 129.70, 137.14, 156.80, 163.11 (4C, C═O).

FD mass spectrum (8 kV): m/z (%): calcd for C₃₀H₂₄Br₄N₂O₄: 796.15. found: 796.4 (100) [M]⁺.

### Example 12.2

### Preparation of 2,6-bis(2,6-diacetyl-4-(tert-butyl)phenyl)pyrrolo[3,4-f]isoindole-1,3,5,7(2H,6H)-tetraone

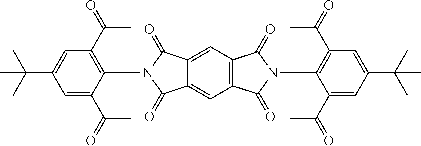 [SMILES: CC(=O)c1cc(C(C)(C)C)cc(C(C)=O)c1-n1c(=O)c2cc3c(=O)n(-c4c(C(C)=O)cc(C(C)(C)C)cc4C(C)=O)c(=O)c3cc2c1=O]

The title compound was prepared in analogy to the method described in example 11.2.

Yield: 56%.

¹H NMR (300 MHz, CD₂Cl₂, 298K): δ 1.45 (s, 18H), 2.57 (s, 12H), 8.00 (s, 4H), 8.35 (s, 2H).

¹³C NMR (75 MHz, CD₂Cl₂, 298K): δ 29.20 (4C, CH₃), 31.18 (6C, CH₃), 35.49 (2C, C—(CH₃)₃), 119.49, 123.90, 129.63, 137.87, 138.06, 153.85, 165.98 (4C, C═O), 199.34 (4C, C═O).

FD mass spectrum (8 kV): m/z (%): calc. for C₃₈H₃₆N₂O₈: 648.71. found: 648.7 (100) [M]⁺.

### Example 12.3

### Preparation of

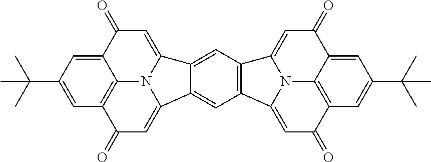 [SMILES: CC(C)(C)c1cc2c(=O)cc3c4cc5c(cc4c4cc(=O)c(c1)c2n34)c1cc(=O)c2cc(C(C)(C)C)cc3c(=O)cc5n1c32]

The title compound was prepared in analogy to the method described in example 11.3.

Yield: 41%.

¹H NMR (300 MHz, D₂SO₄, 298K): δ 2.03 (s, 18H), 8.66 (s, 4H), 9.42 (s, 2H), 9.85 (s, 4H).

¹³C NMR (75 MHz, D₂SO₄, 298K): δ 30.41 (6C, CH₃), 37.19 (2C, C—(CH₃)₃), 111.17, 119.48, 133.20, 139.07, 144.66, 145.37, 152.14, 159.72, 181.26 (4C, C═O).

FD mass spectrum (8 kV): m/z (%): calc. for C₃₈H₂₈N₂O₄: 576.65. found: 576.6 (100) [M]⁺.

### Example 13

### Example 13.1

### Preparation of 2,6-bis(2,6-dibromo-4-decylphenyl)pyrrolo[3,4-f]isoindole-1,3,5,7(2H,6H)-tetraone

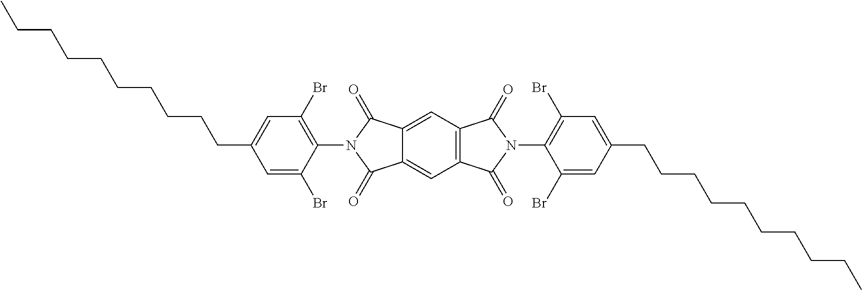 [SMILES: CCCCCCCCCCc1cc(Br)c(-n2c(=O)c3cc4c(=O)n(-c5c(Br)cc(CCCCCCCCCC)cc5Br)c(=O)c4cc3c2=O)c(Br)c1]

The title compound was prepared in analogy to the method described in example 11.1.

Yield: 71%.

¹H NMR (300 MHz, CD₂Cl₂, 298K): δ 0.88 (m, 6H), 1.19-1.49 (m, 28H), 1.67 (p, J=7.3 Hz, 4H), 2.67 (m, 4H), 7.58 (s, 4H), 8.53 (s, 2H).

¹³C NMR (75 MHz, CD₂Cl₂, 298K): δ 14.47 (2C, CH₃), 23.27 (2C, CH₂), 29.77 (2C, CH₂), 29.90 (2C, CH₂), 29.95 (2C, CH₂), 30.09 (2C, CH₂), 30.18 (2C, CH₂), 31.42 (2C, CH₂), 32.49 (2C, CH₂), 35.81 (2C, CH₂), 120.42, 124.62, 128.22, 133.12, 137.80, 149.51, 164.18 (4C, C═O).

FD mass spectrum (8 kV): m/z (%): calc. for C₄₂H₄₈Br₄N₂O₄: 964.47. found: 964.6 (100) [M]⁺.

### Example 13.2

### Preparation of 2,6-bis(2,6-diacetyl-4-decylphenyl)pyrrolo[3,4-f]isoindole-1,3,5,7(2H,6H)-tetraone

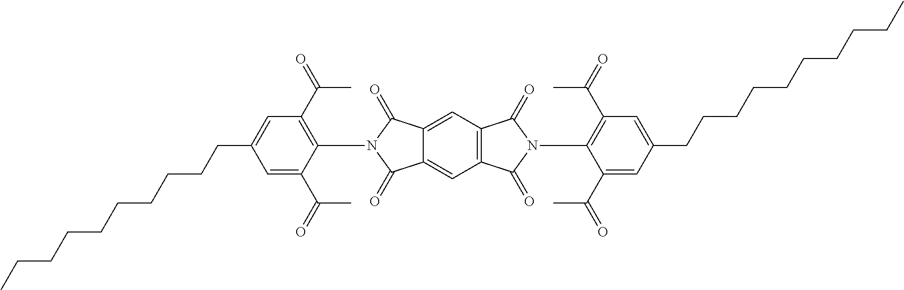 [SMILES: CCCCCCCCCCc1cc(C(C)=O)c(-n2c(=O)c3cc4c(=O)n(-c5c(C(C)=O)cc(CCCCCCCCCC)cc5C(C)=O)c(=O)c4cc3c2=O)c(C(C)=O)c1]

The title compound was prepared in analogy to the method described in example 11.2.

Yield: 86%.

¹H NMR (300 MHz, CD₂Cl₂, 298K): δ 0.88 (m, 6H), 1.30 (m, 28H), 1.73 (m, 4H), 2.56 (s, 12H), 2.81 (m, 4H), 7.82 (s, 4H), 8.35 (s, 2H).

¹³C NMR (75 MHz, CD₂Cl₂, 298K): δ 14.45 (2C, CH₃), 23.27 (2C, CH₂), 29.40 (4C, CH₃), 29.88 (2C, CH₂), 29.90 (2C, CH₂), 30.01 (2C, CH₂), 30.13 (2C, CH₂), 30.19 (2C, CH₂), 31.78 (2C, CH₂), 32.48 (2C, CH₂), 36.21 (2C, CH₂), 119.64, 124.31, 132.78, 138.06, 138.28, 146.10, 166.18 (4C, C═O), 199.31 (4C, C═O).

FD mass spectrum (8 kV): m/z (%): calc. for C₅₀H₆₀N₂O₈: 817.04. found: 817.7 (100) [M]⁺.

### II. Method for Determining the Transistor Characteristics

### Fabrication Procedure:

Highly doped silicon wafers coated with a 30 nm layer of Al₂O₃ prepared by atomic layer deposition (ALD) were thoroughly cleaned by treatment with isopropanol dried at 100° C. at ambient air on a hotplate for 10 min. The surface of the Al₂O₃ layer is treated by a brief exposure to an oxygen plasma. The substrate is then immersed into a 2-propanol solution of an alkyl phosphonic acid (0.34 mg/mL solution of C₁₀H₂₁PO(OH)₂), which results in the formation of a self-assembled monolayer (SAM) on the surface. The highly doped silicon is used as substrate and back gate electrode, the alkyl phosphonic acid treated Al₂O₃ acts as the gate dielectric. A 30 nm thick film of the organic semiconductors from examples 1.3 and 2.4 is deposited on the substrate at a pressure of 7×10⁻⁷ mbar and with an evaporation rate between 0.1 and 0.5 Å/s while the substrate was held at a defined temperature. Gold source-drain contacts were defined with a shadow mask. The channel width (w) is 500 and channel length (l) is 50 μm.

The electrical characteristics of the transistors are measured on a home-build probe station using an Agilent 4156C semiconductor parameter analyzer. All measurements are performed in air at room temperature in the dark. The probe needles are brought into contact with the source and drain contacts of the transistors by putting them down carefully on top of the gold contacts. The gate electrode is contacted by breaking the gate dielectric at a certain position of the chip and pressing a probe needle onto the broken position.

Table 1 gives the field-effect mobilities (p) and on/off ratios (Iₒₙ/I_{off}) for compounds of examples 1.3, 2.4 and 7.4 at a certain substrate temperature (T_{sub}) measured in ambient air.

TABLE 1

| | Substrate | | |
|---|---|---|---|
| Compound | Temperature | Mobility | On/Off Ratio |
| from Example | T_{sub} [° C.] | μ [cm²/Vs] | Iₒₙ/I_{off} |
| 1.3 | 110 | 0.0026 | 7 × 10³ |
| 2.4 | 90 | 0.012 | 10⁴ |
| 7.4 | 80 | 0.07 | 5 × 10⁴ |

### III) Production of the Colored Sample Sheets

### III.1) Soft PVC

### Premix:

0.08 g of compound of formula I was mixed for 30 minutes at room temperature with a mixer with 14.0 g of a base mixture and then slowly stirred with 26.0 g of polyvinyl chloride (PVC) (EVI POL® SH 7060, EVC GmbH). The base mixture consists of plastiziser (12.9 g Palatinol® 10P (di-2-propylheptylphthalate, BASF), 0.6 g Drapex® 39 (epoxidised soya bean oil, Witco Vinyl Additives GmbH) and 0.5 g Mark BZ 561 (Barium/zinc stabilizer, Chemtura GmbH).

Production of Rolled Sheets: The mixture of PVC and compound of formula (1)/base mixture obtained above was rolled in a 2-roll mill (Colin model W110P, D-85560 Ebersberg) at a roll temperature of 160° C. (each roll) in accordance with the following: first cold-rolling for 20 times then hot-rolling for 6 minutes (rolled sheet turned every minute, roll nip 0.35 mm). This gave a milled sheet of thickness 0.33 to 0.35 mm.

Color measurements on the samples were performed according to DIN 53236, method A. All color measurements in remission/transmission are effected using a Minolta CM 3610d spectrophotometer (d/8 geometry, including the gloss, illuminant D65, observer 10°) and B&W Leneta cards. All “angle-depending” measurements are effected using a Datacolor FX 10 and B&W Leneta cards. The Cielab Color System according to DIN 6174 was used to evaluate these results.

### B) Testing by Colorimetry

| | Sample 0.2% by weight of compound I in | | | | |
|---|---|---|---|---|---|
| | soft-PVC prepared as described under A.2 | | | | |
| | L* | a* | b* | C* | h° |
| compound of example 3.3 | 61.74 | 4.79 | −12.63 | 13.51 | 290.77 |

### III.2) PMMA

1000.00 g of polymethyl methacrylate (PPMA 6N clear, available from Röhm GmbH, Germany) were predried at a maximum temperature of 90° C. for 4 hours and then mixed with 0.5 g of a compound of formula I in a Turbula Fuchs mixer for 20 min. The homogenous mixture was extruded on a Twin Screw 25 mm extruder from Collin, Germany, six heating zones (cold, 150° C., 195° C., 200° C., 200° C., 200° C., 200° C.) at a maximum temperature of 200° C. The extrudate was granulated in a granulator (Scheer, Stuttgart). The granulate was dried at a maximum temperature of 90° C. for 4 hours and then processed to colored sample sheets (30 mm×55 mm×1.5 mmm) using a Boy Injection Molding Machine (Boy 30A from Dr. Boy GmbH, Neustadt, Germany) or a Klöckner Ferromatik FM 40 (from Klöckner, Germany). The mouldings were obtained were packed up in an oxygen free plastic bag with a vacuum pack machine after drying. The colored sample sheets were then subjected to colorimetry.

1.-32. (canceled)

33. A compound of the general formula I 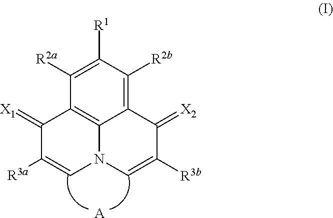
wherein
X¹ and X² are independently of one another selected from O and C(CN)₂;
R¹, R^{2a}, R^{2b}, R^{3a} and R^{3b} are independently of one another selected from hydrogen, F, Cl, Br, I, CN, hydroxy, mercapto, nitro, cyanato, thiocyanato, formyl, acyl, carboxy, carboxylate, alkylcarbonyloxy, carbamoyl, alkylaminocarbonyl, dialkylaminocarbonyl, sulfo, sulfonate, sulfoamino, sulfamoyl, alkylsulfonyl, arylsulfonyl, amidino, NE¹E², where E¹ and E² are each independently selected from hydrogen, alkyl, cycloalkyl, heterocycloalkyl, aryl or hetaryl,
in each case unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, heterocycloalkyl, heterocycloalkoxy, heterocycloalkylthio, (monoheterocycloalkyl)amino, (diheterocycloalkyl)amino, aryl, aryloxy, arylthio, (monoaryl)amino, (diaryl)amino, hetaryl, hetaryloxy, hetarylthio, (monohetaryl)amino and (dihetaryl)amino;
A is selected from the groups of the general formulae (II.1), (II.2), (II.3), (II.4), (II.5) and (II.6) 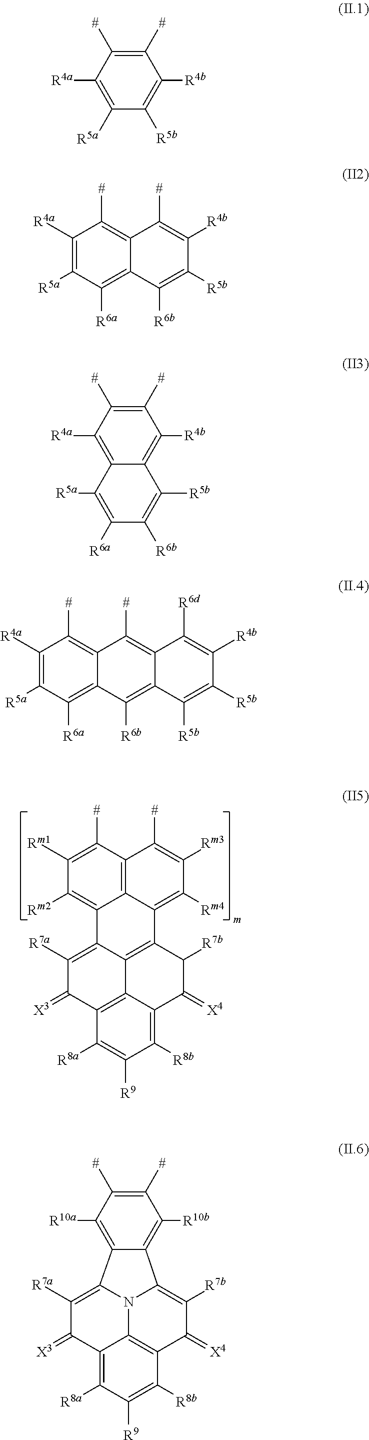
in which
# in each case represents the bonding site to the cyclazine skeleton;
in the formulae (II.1), (II.2), (II.3), (II.4)
R^{4a}, R^{4b}, R^{5a}, R^{5b}, and, if present, R^{6a}, R^{6b}, R^{6c} and R^{6d} are independently of one another selected from hydrogen, F, Cl, Br, I, CN, hydroxy, mercapto, nitro, cyanato, thiocyanato, formyl, acyl, carboxy, carboxylate, alkylcarbonyloxy, carbamoyl, alkylaminocarbonyl, dialkylaminocarbonyl, sulfo, sulfonate, sulfoamino, sulfamoyl, alkylsulfonyl, arylsulfonyl, amidino, NE¹E², where E¹ and E² are each independently selected from hydrogen, alkyl, cycloalkyl, heterocycloalkyl, aryl or hetaryl,
in each case unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, heterocycloalkyl, heterocycloalkoxy, heterocycloalkylthio, (monoheterocycloalkyl)amino, (diheterocycloalkyl)amino, aryl, aryloxy, arylthio, (monoaryl)amino, (diaryl)amino, hetaryl, hetaryloxy, hetarylthio, (monohetaryl)amino and (dihetaryl)amino;
m in the formula (II.5) is 1, 2, 3 or 4;
in the formulae (II.5) and (II.6)
X³ and X⁴ are independently of one another selected from O and C(CN)₂;
R^{7a}, R^{7b}, R^{8a}, R^{8b}, R⁹ and, if present, each R^{m1}, R^{m2}, R^{m3} and R^{m4}, are independently of one another selected from hydrogen, F, Cl, Br, I, CN, hydroxy, mercapto, nitro, cyanato, thiocyanato, formyl, acyl, carboxy, carboxylate, alkylcarbonyloxy, carbamoyl, alkylaminocarbonyl, dialkylaminocarbonyl, sulfo, sulfonate, sulfoamino, sulfamoyl, alkylsulfonyl, arylsulfonyl, amidino, NE¹E², where E¹ and E² are each independently selected from hydrogen, alkyl, cycloalkyl, heterocycloalkyl, aryl or hetaryl,
in each case unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, heterocycloalkyl, heterocycloalkoxy, heterocycloalkylthio, (monoheterocycloalkyl)amino, (diheterocycloalkyl)amino, aryl, aryloxy, arylthio, (monoaryl)amino, (diaryl)amino, hetaryl, hetaryloxy, hetarylthio, (monohetaryl)amino and (dihetaryl)amino;
R^{10a} and R^{10b}, if present, are independently of one another selected from hydrogen, F, Cl, Br, I, CN, hydroxy, mercapto, nitro, cyanato, thiocyanato, formyl, acyl, carboxy, carboxylate, alkylcarbonyloxy, carbamoyl, alkylaminocarbonyl, dialkylaminocarbonyl, sulfo, sulfonate, sulfoamino, sulfamoyl, alkylsulfonyl, arylsulfonyl, amidino, NE¹E², where E¹ and E² are each independently selected from hydrogen, alkyl, cycloalkyl, heterocycloalkyl, aryl and hetaryl,
in each case unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, heterocycloalkyl, heterocycloalkoxy, heterocycloalkylthio, (monoheterocycloalkyl)amino, (diheterocycloalkyl)amino, aryl, aryloxy, arylthio, (monoaryl)amino,
(diaryl)amino, hetaryl, hetaryloxy, hetarylthio, (monohetaryl)amino and (dihetaryl)amino.

34. The compound according to claim 33, in which A is selected from groups of the general formulae (II.1), (II.2), (II.3) and (II.4).

35. The compound according to claim 33, in which A is selected from the groups of the general formulae (II.1), (II.2), (II.3), (II.4) and (II.5).

36. The compound of the general formula I according to claim 33 which is selected from compounds of the formulae (I.A) and (I.B) 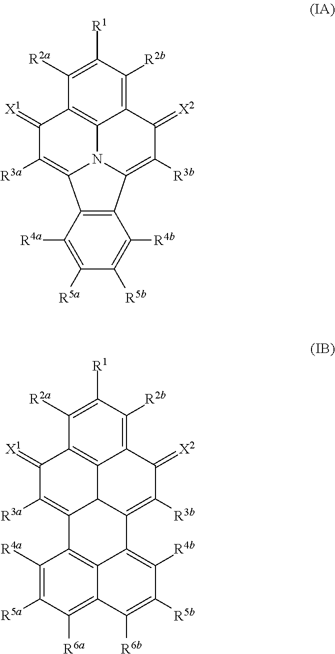
wherein
R^{4a}, R^{4b}, R^{5a}, R^{5b}, and, if present, R^{6a} and R^{6b} are independently of one another selected from hydrogen, F, Cl, Br, I, CN, hydroxy, mercapto, nitro, cyanato, thiocyanato, formyl, acyl, carboxy, carboxylate, alkylcarbonyloxy, carbamoyl, alkylaminocarbonyl, dialkylaminocarbonyl, sulfo, sulfonate, sulfoamino, sulfamoyl, alkylsulfonyl, arylsulfonyl, amidino, NE¹E², where E¹ and E² are each independently selected from hydrogen, alkyl, cycloalkyl, heterocycloalkyl, aryl or hetaryl,
in each case unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, heterocycloalkyl, heterocycloalkoxy, heterocycloalkylthio, (monoheterocycloalkyl)amino,
(diheterocycloalkyl)amino, aryl, aryloxy, arylthio, (monoaryl)amino, (diaryl)amino, hetaryl, hetaryloxy, hetarylthio, (monohetaryl)amino and (dihetaryl)amino.

37. The compound of the general formula I according to claim 33 which is selected from compounds of the formula (I.C) 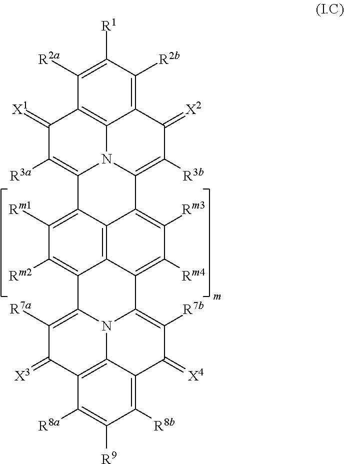
wherein
m is 1, 2, 3 or 4;
X¹, X², X³ and X⁴ are independently of one another selected from O and C(CN)₂;
R¹, R^{2a}, R^{2b}, R^{3a}, R^{3b}, R^{7a}, R^{7b}, R^{8a}, R^{8b}, R⁹ and each R^{m1}, R^{m2}, R^{m3} and R^{m4} are independently of one another selected from hydrogen, F, Cl, Br, I, CN, hydroxy, mercapto, nitro, cyanato, thiocyanato, formyl, acyl, carboxy, carboxylate, alkylcarbonyloxy, carbamoyl, alkylaminocarbonyl, dialkylaminocarbonyl, sulfo, sulfonate, sulfoamino, sulfamoyl, alkylsulfonyl, arylsulfonyl, amidino, NE¹E², where E¹ and E² are each independently selected from hydrogen, alkyl, cycloalkyl, heterocycloalkyl, aryl or hetaryl,
in each case unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, heterocycloalkyl, heterocycloalkoxy, heterocycloalkylthio, (monoheterocycloalkyl)amino, (diheterocycloalkyl)amino, aryl, aryloxy, arylthio, (monoaryl)amino, (diaryl)amino, hetaryl, hetaryloxy, hetarylthio, (monohetaryl)amino and (dihetaryl)amino.

38. The compound of the general formula I according to claim 33 which is selected from compounds of the formula (I.D) 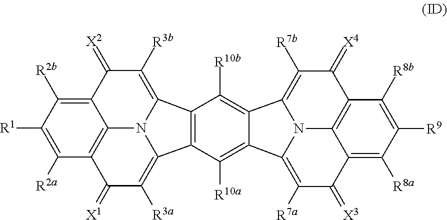
wherein
X¹, X², X³ and X⁴ are independently of one another selected from O and C(CN)₂;
R¹, R^{2a}, R^{2b}, R^{3a}, R^{3b}, R^{7a}, R^{7b}, R^{8a}, R^{8b}, R⁹, R^{10a} and R^{10b} are independently of one another selected from hydrogen, F, Cl, Br, I, CN, hydroxy, mercapto, nitro, cyanato, thiocyanato, formyl, acyl, carboxy, carboxylate, alkylcarbonyloxy, carbamoyl, alkylaminocarbonyl, dialkylaminocarbonyl, sulfo, sulfonate, sulfoamino, sulfamoyl, alkylsulfonyl, arylsulfonyl, amidino, NE¹E², where E¹ and E² are each independently selected from hydrogen, alkyl, cycloalkyl, heterocycloalkyl, aryl and hetaryl,
in each case unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, heterocycloalkyl, heterocycloalkoxy, heterocycloalkylthio, (monoheterocycloalkyl)amino, (diheterocycloalkyl)amino, aryl, aryloxy, arylthio, (monoaryl)amino, (diaryl)amino, hetaryl, hetaryloxy, hetarylthio, (monohetaryl)amino and (dihetaryl)amino.

39. The compound of the general formula I according to claim 33, wherein
X¹ and X² are independently of one another selected from O and C(CN)₂;
R¹, R^{2a}, R^{2b}, R^{3a} and R^{3b} are independently of one another selected from hydrogen, F, Cl, Br, I, CN, hydroxy, mercapto, NE¹E², where E¹ and E² are each independently selected from hydrogen, alkyl, cycloalkyl and aryl,
in each case unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, aryl, aryloxy, arylthio, (monoaryl)amino and (diaryl)amino;
A is selected from the groups of the general formulae (II.1), (II.2), (II.3), (II.4), (II.5) and (II.6) 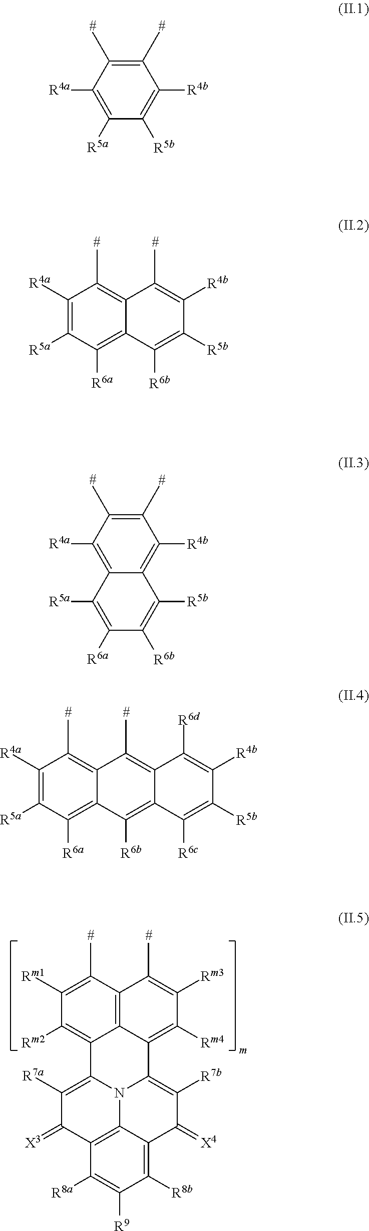 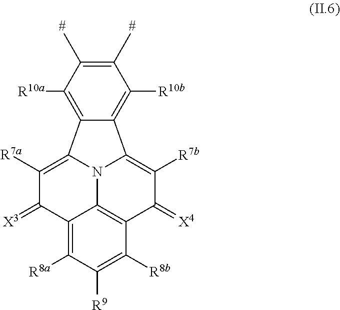
in which
# in each case represents the bonding site to the cyclazine skeleton;
in the formulae (II.1), (II.2), (II.3), (II.4)
R^{4a}, R^{4b}, R^{5a}, R^{5b}, and, if present, R^{6a}, R^{6b}, R^{6c} and R^{6d} are independently of one another selected from hydrogen, F, Cl, Br, I, CN, hydroxy, mercapto, formyl, acyl, carboxy, carboxylate, alkylcarbonyloxy, sulfo, sulfonate, sulfoamino, alkylsulfonyl, arylsulfonyl, NE¹E², where E¹ and E² are each independently selected from hydrogen, alkyl, cycloalkyl and aryl,
in each case unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, aryl, aryloxy, arylthio, (monoaryl)amino and (diaryl)amino;
m in the formula (II.5) is 1, 2, 3 or 4;
in the formulae (II.5) and (II.6)
X³ and X⁴ are independently of one another selected from O and C(CN)₂;
R^{7a}, R^{7b}, R^{8a}, R^{8b}, R⁹ and, if present, each R^{m1}, R^{m2}, R^{m3} and R^{m4} are independently of one another selected from hydrogen, F, Cl, Br, I, CN, hydroxy, mercapto, NE¹E², where E¹ and E² are each independently selected from hydrogen, alkyl, cycloalkyl and aryl,
in each case unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, aryl, aryloxy, arylthio, (monoaryl)amino and (diaryl)amino;
R^{10a}, R^{10b}, if present, are independently of one another selected from hydrogen, F, Cl, Br, I, CN, hydroxy, mercapto, formyl, acyl, carboxy, carboxylate, alkylcarbonyloxy, sulfo, sulfonate, sulfoamino, alkylsulfonyl, arylsulfonyl, NE¹E², where E¹ and E² are each independently selected from hydrogen, alkyl, cycloalkyl and aryl
in each case unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, aryl, aryloxy, arylthio, (monoaryl)amino and (diaryl)amino.

40. The compound according to claim 33, wherein the radicals R¹, R^{2a}, R^{2b}, R^{3a}, R^{3b}, and, if present, R^{4a}, R^{4b}, R^{5a}, R^{5b}, R^{6a}, R^{6b}, R^{6c}, R^{6d}, R^{7a}, R^{7b}, R^{8a}, R^{8b}, R⁹, R^{10a}, R^{10b} and each R^{m1}, R^{m2}, R^{m3} and R^{m4} are independently of one another selected from hydrogen, linear C₁-C₃₀-alkyl, branched C₃-C₃₀-alkyl, perfluoro-C₁-C₃₀-alkyl, 1H,1H-perfluoro-C₂-C₃₀-alkyl, 1H,1H,2H,2H-perfluoro-C₃-C₃₀-alkyl, a radical of the formula G.1, a radical of the formula G.2 and a radical of the formula G.3 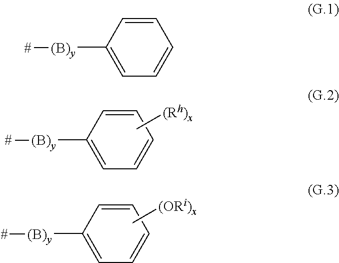
where
# represents the bonding side to the remainder of the molecule;
B where present, is selected from O, S and a C₁-C₁₀-alkylene group which may be interrupted by one or more nonadjacent groups which are selected from O and S;
y is 0 or 1;
R^{h} is independently of one another selected from C₁-C₃₀-alkyl, C₁-C₃₀-fluoroalkyl, fluorine, chlorine, bromine, NE³E⁴, nitro and cyano, where E³ and E⁴, independently of one another, are hydrogen, alkyl, cycloalkyl, heterocycloalkyl, aryl or hetaryl;
Rⁱ is independently of one another selected from C₁-C₃₀-alkyl;
x in formulae G.2 and G.3 is 1, 2, 3, 4 or 5.

41. The compound according to claim 33, wherein X¹ and X² have the same meaning.

42. The compound of the formula (I) according to claim 33, wherein A is a group of the formulae (II.5) or (II.6), wherein X¹, X², X³ and X⁴ have the same meaning.

43. A compound according to claim 33, wherein R¹ and, if present, R⁹ are selected from radicals of the general formulae (III.1) and (III.2) 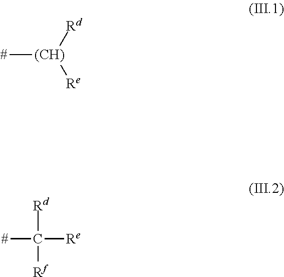
in which
# is a bonding site; and
in the formula (III.1) R^{d} and R^{e} are independently selected from C₁- to C₂₈-alkyl, where the sum of the carbon atoms of the R^{d} and R^{e} radicals is an integer from 2 to 29,
in the formula (III.2) R^{d}, R^{e} and R^{f} are independently selected from C₁- to C₂₇-alkyl, where the sum of the carbon atoms of the R^{d}, R^{e} and R^{f} radicals is an integer from 3 to 29.

44. The compound according to claim 33, wherein R¹ and, if present, R⁹ are isopropyl or tert.-butyl.

45. The compound according to claim 33, wherein R¹ and, if present, R⁹ are hydrogen or linear C₁-C₁₀-alkyl.

46. The compound according to claim 33, wherein R^{3a}, R^{3b}, and, if present, R^{7a} and R^{7b} are independently selected from hydrogen, unsubstituted or substituted alkyl and unsubstituted or substituted aryl.

47. The compound according to claim 33, wherein R^{2a}, R^{2b}, R^{3a}, R^{3b}, and, if present, R^{4a}R^{4b}, R^{5a}, R^{5b}, R^{6a}, R^{6b}, R^{6c}, R^{6d}, R^{7a}, R^{7b}, R^{8a}, R^{8b}, R⁹, R^{10a}, R^{10b} and each R^{m1}, R^{m2}, R^{m3} and R^{m4} are all hydrogen.

48. The compound of the general formula I according to claim 33 which is selected from compounds of the formula (I.Ca) 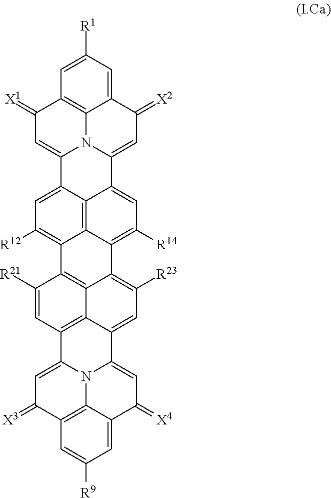
wherein
X¹, X², X³ and X⁴ are all O or all C(CN)₂;
R¹ and R⁹ are independently of one another selected from hydrogen, F, Cl, Br, I, CN, hydroxy, mercapto, nitro, cyanato, thiocyanato, formyl, acyl, carboxy, carboxylate, alkylcarbonyloxy, carbamoyl, alkylaminocarbonyl, dialkylaminocarbonyl, sulfo, sulfonate, sulfoamino, sulfamoyl, alkylsulfonyl, arylsulfonyl, amidino, NE¹E², where E¹ and E² are each independently selected from hydrogen, alkyl, cycloalkyl, heterocycloalkyl, aryl or hetaryl,
in each case unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, heterocycloalkyl, heterocycloalkoxy, heterocycloalkylthio, (monoheterocycloalkyl)amino, (diheterocycloalkyl)amino, aryl, aryloxy, arylthio, (monoaryl)amino, (diaryl)amino, hetaryl, hetaryloxy, hetarylthio, (monohetaryl)amino and (dihetaryl)amino, and
R¹⁴ and R²¹ are both hydrogen and R¹² and R²³ have the same meaning and are selected from F, Cl, Br, I, CN and in each case unsubstituted or substituted aryl, aryloxy and arylthio,
or R²¹ and R²³ are both hydrogen and R¹² and R¹⁴ have the same meaning and are selected from F, Cl, Br, I, CN and in each case unsubstituted or substituted aryl, aryloxy and arylthio,
or R¹², R¹⁴, R²¹ and R²³ have the same meaning and are selected from hydrogen, F, Cl, Br, I, CN and in each case unsubstituted or substituted aryl, aryloxy and arylthio.

49. A process for the preparation of the compound of the formula I.A as claimed in claim 36,
wherein
X¹ and X² are independently of one another selected from O and C(CN)₂;
R¹, R^{2a}, R^{2b}, R^{4a}, R^{4b}, R^{5a} and R^{5b} are independently of one another selected from hydrogen, F, Cl, Br, I, CN, hydroxy, mercapto, nitro, cyanato, thiocyanato, formyl, acyl, carboxy, carboxylate, alkylcarbonyloxy, carbamoyl, alkylaminocarbonyl, dialkylaminocarbonyl, sulfo, sulfonate, sulfoamino, sulfamoyl, alkylsulfonyl, arylsulfonyl, amidino, NE¹E², where E¹ and E² are each independently selected from hydrogen, alkyl, cycloalkyl, heterocycloalkyl, aryl or hetaryl,
in each case unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, heterocycloalkyl, heterocycloalkoxy, heterocycloalkylthio, (monoheterocycloalkyl)amino, (diheterocycloalkyl)amino, aryl, aryloxy, arylthio, (monoaryl)amino, (diaryl)amino, hetaryl, hetaryloxy, hetarylthio, (monohetaryl)amino and (dihetaryl)amino;
comprising
i.a) reacting a phthalic anhydride compound (A) and a 2,6-dibromoaniline compound (B1) to obtain an imide compound of the formula (C) 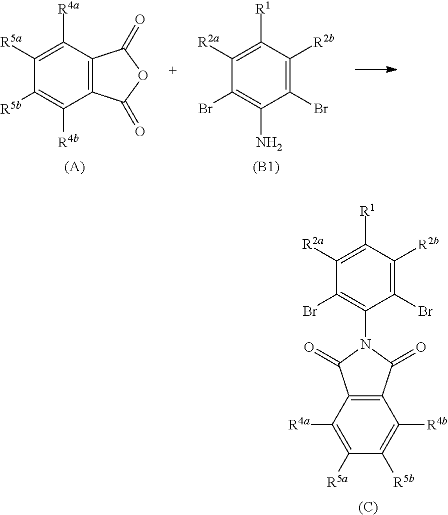
ii.a) reacting the compound of the formula (C) with tributyl(l-ethoxy-1-ethenyl)stannane in the presence of a Pd catalyst to obtain a compound (D) 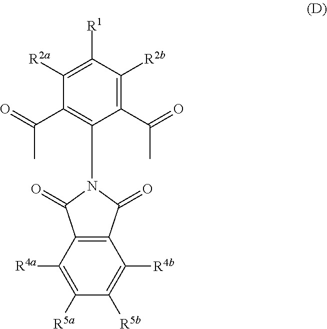
iii.a) subjecting the compound of the formula (D) to a condensation reaction to obtain a compound (I.A1) 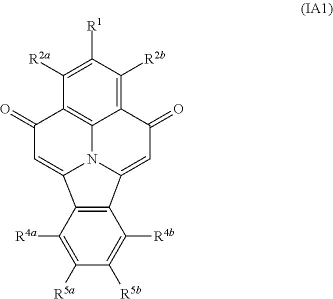
iv.a) optionally reacting the compound of the formula (I.A1) with malononitrile to obtain a compound (I.A2)

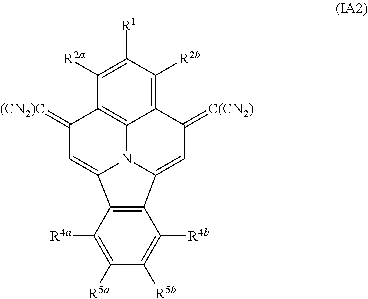

50. A process for the preparation of the compound of the formula I.B as claimed in claim 36,
wherein
X¹ and X² are independently of one another selected from O and C(CN)₂;
R¹, R^{2a}, R^{2b}, R^{4a}, R^{4b}, R^{5a}, R^{5b}, R^{6a} and R^{6b} are independently of one another selected from hydrogen, F, Cl, Br, I, CN, hydroxy, mercapto, nitro, cyanato, thiocyanato, formyl, acyl, carboxy, carboxylate, alkylcarbonyloxy, carbamoyl, alkylaminocarbonyl, dialkylaminocarbonyl, sulfo, sulfonate, sulfoamino, sulfamoyl, alkylsulfonyl, arylsulfonyl, amidino, NE¹E², where E¹ and E² are each independently selected from hydrogen, alkyl, cycloalkyl, heterocycloalkyl, aryl or hetaryl,
in each case unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, heterocycloalkyl, heterocycloalkoxy, heterocycloalkylthio, (monoheterocycloalkyl)amino, (diheterocycloalkyl)amino, aryl, aryloxy, arylthio, (monoaryl)amino, (diaryl)amino, hetaryl, hetaryloxy, hetarylthio, (monohetaryl)amino and (dihetaryl)amino,
comprising
i.b) reacting a 2,6-dibromoaniline (B1) with tributyl(1-ethoxy-1-ethenyl)stannane in the presence of a Pd catalyst to obtain a compound (E1) 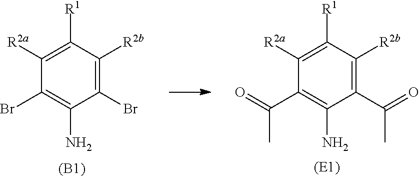
ii.b) reacting the compound of the formula (E1) with a naphthalene monoanhydride (F) to obtain a compound (I.B1) 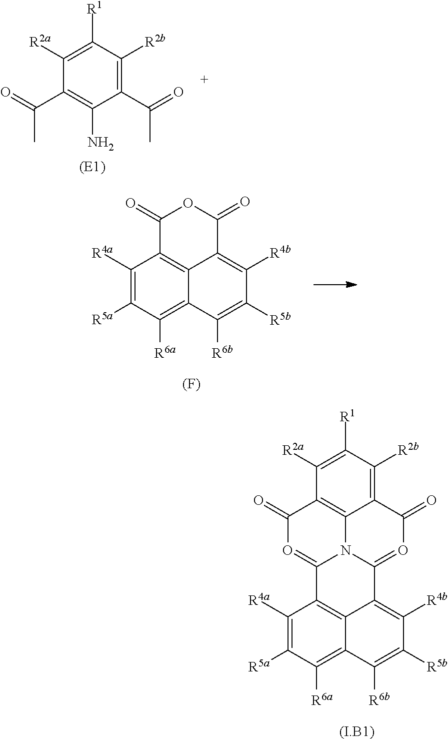
iii.b) optionally reacting the compound of the formula (I.B1) with malononitrile to obtain a compound (I.B2)

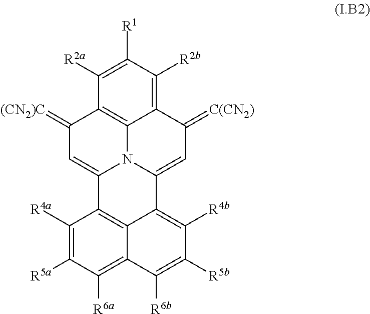

51. A process for the preparation of the compound of the formula I.C as claimed in claim 37,
wherein
m is 1, 2, 3 or 4;
X¹, X², X³ and X⁴ are independently of one another selected from O and C(CN)₂;
R¹, R^{2a}, R^{2b}, R^{8a}, R^{8b}, R⁹ and each R^{m1}, R^{m2}, R^{m3} and R^{m4} are independently of one another selected from hydrogen, F, Cl, Br, I, CN, hydroxy, mercapto, nitro, cyanato, thiocyanato, formyl, acyl, carboxy, carboxylate, alkylcarbonyloxy, carbamoyl, alkylaminocarbonyl, dialkylaminocarbonyl, sulfo, sulfonate, sulfoamino, sulfamoyl, alkylsulfonyl, arylsulfonyl, amidino, NE¹E², where E¹ and E² are each independently selected from hydrogen, alkyl, cycloalkyl, heterocycloalkyl, aryl and hetaryl,
in each case unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, heterocycloalkyl, heterocycloalkoxy, heterocycloalkylthio, (monoheterocycloalkyl)amino, (diheterocycloalkyl)amino, aryl, aryloxy, arylthio, (monoaryl)amino,
(diaryl)amino, hetaryl, hetaryloxy, hetarylthio, (monohetaryl)amino and (dihetaryl)amino,
comprising
i.c) reacting a rylene tetracarboxylic acid bisanhydride (K) 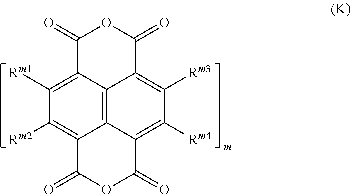
with a 2,6-dibromoaniline of the formula (B1) 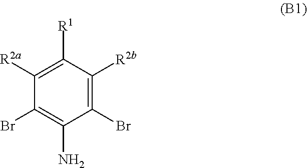
and optionally a different 2,6-dibromoaniline of the formula (B2) 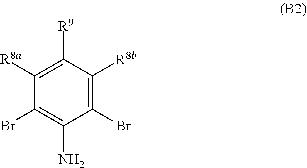
to obtain an imide compound of the formula (L) 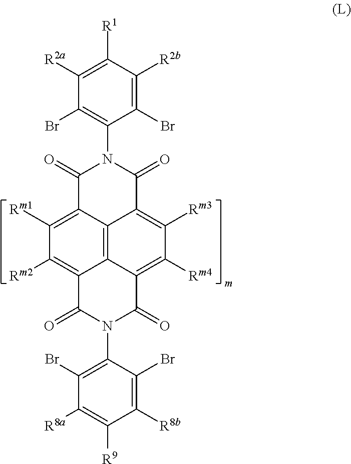
ii.c) reacting the compound of the formula (L) with tributyl(l-ethoxy-1-ethenyl)stannane in the presence of a Pd catalyst to obtain a compound (M) 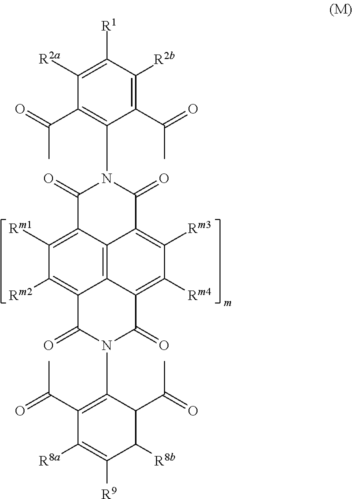
iii.c) subjecting the compound of the formula (M) to a condensation reaction to obtain a compound (I.C1) 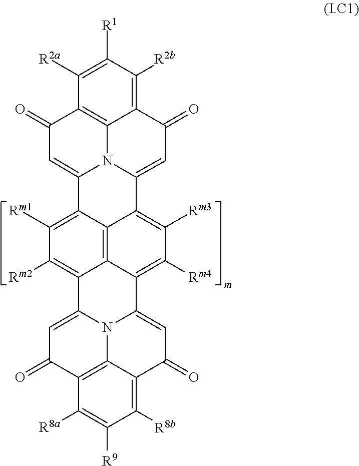
iv.c) optionally reacting the compound of the formula (I.C1) with malononitrile to obtain a compound (I.C2)

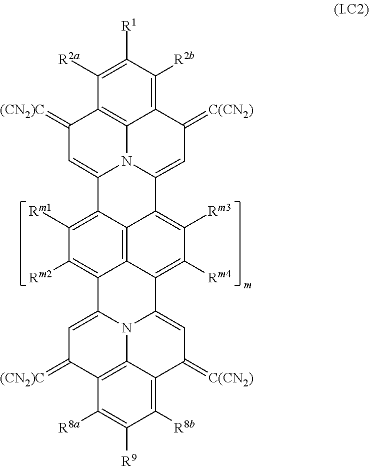

52. A process for the preparation of the compound of the formula I.C as claimed in claim 37,
wherein
m is 1, 2, 3 or 4;
X¹, X², X³ and X⁴ are independently of one another selected from O and C(CN)₂;
R¹, R^{2a}, R^{2b}, R^{8a}, R^{8b}, R⁹ and each R^{m1}, R^{m2}, R^{m3} and R^{m4} are independently of one another selected from hydrogen, F, Cl, Br, I, CN, hydroxy, mercapto, nitro, cyanato, thiocyanato, formyl, acyl, carboxy, carboxylate, alkylcarbonyloxy, carbamoyl, alkylaminocarbonyl, dialkylaminocarbonyl, sulfo, sulfonate, sulfoamino, sulfamoyl, alkylsulfonyl, arylsulfonyl, amidino, NE¹E², where E¹ and E² are each independently selected from hydrogen, alkyl, cycloalkyl, heterocycloalkyl, aryl and hetaryl,
in each case unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, heterocycloalkyl, heterocycloalkoxy, heterocycloalkylthio, (monoheterocycloalkyl)amino, (diheterocycloalkyl)amino, aryl, aryloxy, arylthio, (monoaryl)amino, (diaryl)amino, hetaryl, hetaryloxy, hetarylthio, (monohetaryl)amino and (dihetaryl)amino;
comprising
i.d) reacting a rylene tetracarboxylic acid bisanhydride of the formula (K) 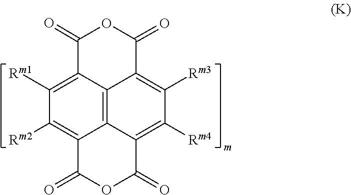
with a compound of the formula (E1) 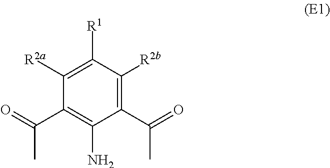
and optionally with a different compound (E2) 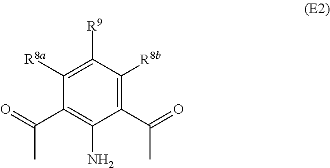
to obtain a compound (I.C1) 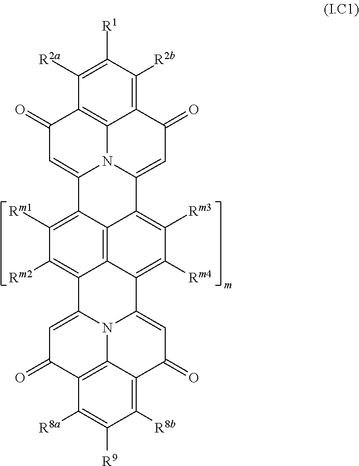
ii.d) optionally reacting the compound of the formula (I.C1) with malononitrile to obtain a compound (I.C2)

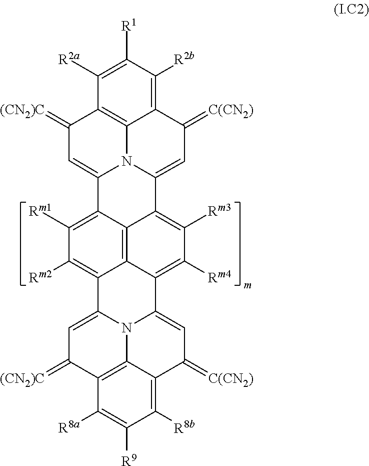

53. A process for the preparation of the compound of the formula I.D as claimed in claim 38,
wherein
X¹, X², X³ and X⁴ are independently of one another selected from O and C(CN)₂;
R¹, R^{2a}, R^{2h}, R^{8a}, R^{8b}, R^{10a} and R^{10b} are independently of one another selected from hydrogen, F, Cl, Br, I, CN, hydroxy, mercapto, nitro, cyanato, thiocyanato, formyl, acyl, carboxy, carboxylate, alkylcarbonyloxy, carbamoyl, alkylaminocarbonyl, dialkylaminocarbonyl, sulfo, sulfonate, sulfoamino, sulfamoyl, alkylsulfonyl, arylsulfonyl, amidino, NE¹E², where E¹ and E² are each independently selected from hydrogen, alkyl, cycloalkyl, heterocycloalkyl, aryl or hetaryl,
in each case unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, heterocycloalkyl, heterocycloalkoxy, heterocycloalkylthio, (monoheterocycloalkyl)amino, (diheterocycloalkyl)amino, aryl, aryloxy, arylthio, (monoaryl)amino, (diaryl)amino, hetaryl, hetaryloxy, hetarylthio, (monohetaryl)amino and (dihetaryl)amino;
comprising
i.e) reacting a pyromellitic dianhydride of the formula (N) 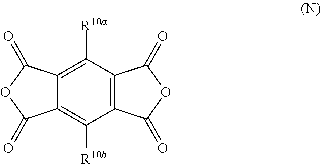
with a 2,6-dibromoaniline of the formula (B1) 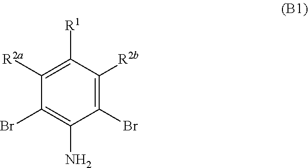
and optionally a different 2,6-dibromoaniline of the formula (B2) 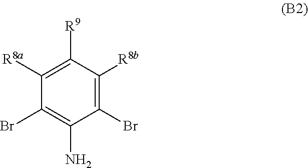
to obtain an imide compound of the formula (O) 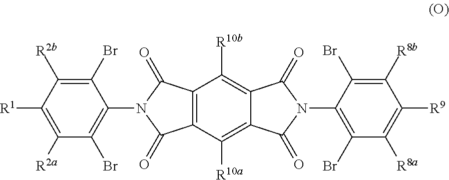
ii.e) reacting the compound of the formula (O) with tributyl(1-ethoxy-1-ethenyl)stannane in the presence of a Pd catalyst to obtain a compound of formula (P) 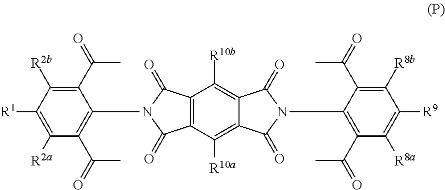
iii.e) subjecting the compound of the formula (P) to a condensation reaction to obtain a compound (I.D1) 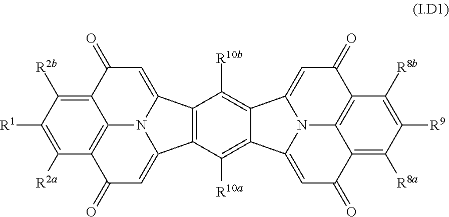
iv.e) optionally reacting the compound of the formula (I.D1) with malononitrile to obtain a compound of the formula (I.D2)

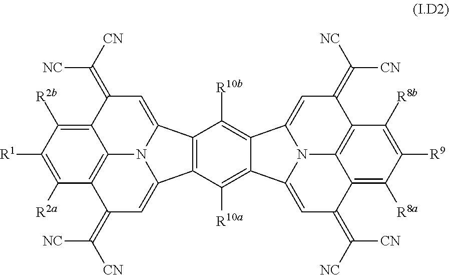

54. An article which comprises at least one compound of the formula (I) as defined in claim 33, wherein the article is
fluorescent colorants,
for data storage,
as a UV absorber,
for optical labels,
as a fluorescent label for biomolecules,
in the laser welding of polymer materials,
in inks,
in surface coatings, and
for coloring polymer compositions.

55. An organic field-effect transistor, comprising a substrate having at least one gate structure, a source electrode and a drain electrode and at least one compound of the formula I as defined in claim 33 as a semiconductor material.

56. A substrate comprising a plurality of organic field-effect transistors, at least some of the field-effect transistors comprising at least one compound of the formula I as defined in claim 33.

57. A semiconductor unit comprising at least one substrate as defined in claim 56.

58. An electroluminescent arrangement comprising an upper electrode, a lower electrode, wherein at least one of said electrodes is transparent, an electroluminescent layer and optionally an auxiliary layer, wherein the electroluminescent arrangement comprises at least one compound of the formula I as defined in claim 33.

59. An electroluminescent arrangement as claimed in claim 58 comprising at least one compound of the formula I as defined in claim 33 in a hole-injecting layer or as part of a transparent electrode.

60. An electroluminescent arrangement as claimed in claim 58 in form of an organic light-emitting diode (OLED).

61. An organic solar cell comprising at least one compound of the formula (I) as defined in claim 33.

62. A composition comprising at least one compound of the formula (I) as defined in claim 33 and at least polymer.

63. The composition according to claim 62, wherein the thermoplastic polymer is selected from
homo- and copolymers which comprise at least one copolymerized monomer selected from C₂-C₁₀-monoolefins, 1,3-butadiene, 2-chloro-1,3-butadiene, vinyl alcohol and its C₂-C₁₀-alkyl esters, vinyl chloride, vinylidene chloride, vinylidene fluoride, tetrafluoroethylene, glycidyl acrylate, glycidyl methacrylate, acrylates and methacrylates of C₁-C₁₀-alcohols, vinylaromatics, (meth)acrylonitrile, maleic anhydride, and α,β-ethylenically unsaturated mono- and dicarboxylic acids,
homo- and copolymers of vinyl acetals,
polyvinyl esters,
polycarbonates,
polyesters,
polyethers,
polyether ketones,
thermoplastic polyurethanes,
polysulfides,
polysulfones,
polyether sulfones,
cellulose alkyl esters,
and mixtures thereof.
